(19) United States
(12) Patent Application Publication
(10) Pub. No.: US 2011/0129456 A1
(43) Pub. Date: Jun. 2, 2011

(54) Sequential Administration of Chemotherapeutic Agents for Treatment of Cancer

(76) Inventors: Yaolin Wang, Edison, NJ (US); Yan Wang, Warren, NJ (US); Brian Der-Hua Lu, Westfield, NJ (US); Ming Liu, Fanwood, NJ (US); Cynthia Seidel-Dugan, Mountainside, NJ (US); Siu-Long Yao, West Windsor, NJ (US)

(21) Appl. No.: 12/991,228

(22) PCT Filed: May 4, 2009
(86) PCT No.: PCT/US09/42657

§ 371 Date: Feb. 21, 2011

Related U.S. Application Data
(60) Provisional application No. 61/050,405, filed on May 5, 2008.

(51) Int. Cl. A61K 39/395; A61P 35/00; A61P 35/02

(52) U.S. Cl. 424/131.1; 424/130.1; 424/141.1; 424/133.1; 424/136.1

(57) ABSTRACT

The present invention relates to the sequential administration of a cytotoxic agent followed by an IGF1R antagonist (e.g., an antibody) for the treatment of hyperproliferative disorders including cancer.

The present application claims the benefit of U.S. provisional patent application No. 61/050,405; filed May 5, 2008, which is herein incorporated by reference in its entirety.

### FIELD OF THE INVENTION

The present invention relates, in general, to methods for treating or prevent a hyperproliferative disorder by administering a cytotoxic agent followed by an IGF1R inhibitor.

### BACKGROUND OF THE INVENTION

The insulin-like growth factors, also known as somatomedins, include insulin-like growth factor-I (IGF-I) and insulin-like growth factor-II (IGF-II) (Klapper, et al., (1983) Endocrinol. 112:2215 and Rinderknecht, et al., (1978) Febs. Lett. 89:283). These growth factors exert mitogenic activity on various cell types, including tumor cells (Macaulay, (1992) Br. J. Cancer 65:311), by binding to a common receptor named the insulin-like growth factor receptor-1 (IGF1R) (Sepp-Lorenzino, (1998) Breast Cancer Research and Treatment 47:235). Interaction of IGFs with IGF1R activates the receptor by triggering autophosphorylation of the receptor on tyrosine residues (Butler, et al., (1998) Comparative Biochemistry and Physiology 121:19). Once activated, IGF1R, in turn, phosphorylates intracellular targets to activate cellular signaling pathways. This receptor activation is critical for stimulation of tumor cell growth and survival. Therefore, inhibition of IGF1R activity represents a valuable method to treat or prevent growth of human cancers and other proliferative diseases.

Combined administration of an IGF1R inhibitor and a cytotoxic agent has been observed to lead to superior clinical results relative to the administration of either alone. However, there is an interest in developing modifications to combination regimens which further enhance clinical outomes.

### SUMMARY OF THE INVENTION

The present invention provides, e.g., methods which modify the treatment regimen of combination of IGF1R inhibitors and cytotoxic agents which lead to enhanced efficacy.

The present invention provides a method for treating or preventing a hyperproliferative disorder (e.g., cancer) mediated by elevated expression or activity of insulin-like growth factor I receptor or elevated expression of IGF-1 or elevated expression of IGF-II, in a subject, comprising first administering a therapeutically effective amount of a cytotoxic anti-cancer chemotherapeutic agent (e.g., irinotecan or cyclophosphamide) to the subject, then administering a therapeutically effective amount of an IGF1R inhibitor (e.g., anti-IGF1R antibody) to the subject. For example, the present invention provides, e.g., a method for treating or preventing a hyperproliferative disorder mediated by elevated expression or activity of insulin-like growth factor I receptor or elevated expression of IGF-1 or elevated expression of IGF-II (e.g., osteosarcoma, rhabdomyosarcoma, neuroblastoma, any pediatric cancer, kidney cancer, leukemia, renal transitional cell cancer, bladder cancer, Wilm's cancer, ovarian cancer, pancreatic cancer, benign prostatic hyperplasia, breast cancer, prostate cancer, bone cancer, lung cancer, gastric cancer, colorectal cancer, cervical cancer, synovial sarcoma, diarrhea associated with metastatic carcinoid, vasoactive intestinal peptide secreting tumors, psoriasis, smooth muscle restenosis of blood vessels and inappropriate microvascular proliferation, head and neck cancer, squamous cell carcinoma, multiple myeloma, solitary plasmacytoma, renal cell cancer, retinoblastoma, germ cell tumors, hepatoblastoma, hepatocellular carcinoma, melanoma, rhabdoid tumor of the kidney, Ewing Sarcoma, chondrosarcoma, haemotological malignancy, chronic lymphoblastic leukemia, chronic myelomonocytic leukemia, acute lymphoblastic leukemia, acute lymphocytic leukemia, acute myelogenous leukemia, acute myeloblastic leukemia, chronic myeloblastic leukemia, Hodgekin's disease, non-Hodgekin's lymphoma, chronic lymphocytic leukemia, chronic myelogenous leukemia, myelodysplastic syndrome, hairy cell leukemia, mast cell leukemia, mast cell neoplasm, follicular lymphoma, diffuse large cell lymphoma, mantle cell lymphoma, Burkitt Lymphoma, mycosis fungoides, seary syndrome, cutaneous T-cell lymphoma, chronic myeloproliferative disorders, a cental nervous system tumor, brain cancer, glioblastoma (e.g., glioblastoma multiforme), non-glioblastoma brain cancer, meningioma, pituitary adenoma, vestibular schwannoma, a primitive neuroectodermal tumor, medulloblastoma, astrocytoma, anaplastic astrocytoma, oligodendroglioma, ependymoma and choroid plexus papilloma, a myeloproliferative disorder, polycythemia vera, thrombocythemia, idiopathic myeifibrosis, soft tissue sarcoma, thyroid cancer, endometrial cancer, carcinoid cancer, germ cell tumors, liver cancer), in a subject (e.g., a mammalian subject such as a human), comprising first administering a therapeutically effective amount of cyclophosphamide or irinotecan to the subject, then administering a therapeutically effective amount of an IGF1R inhibitor to the subject. In an embodiment of the invention, the IGF1R inhibitor is an isolated antibody (e.g., monoclonal antibody, e.g., in a pharmaceutically composition with a pharmaceutically acceptable carrier) or antigen-binding fragment thereof (e.g., a monoclonal antibody, a labeled antibody, bivalent antibody, a polyclonal antibody, a bispecific antibody, a chimeric antibody, a recombinant antibody, an anti-idiotypic antibody, a humanized antibody or a bispecific antibody, camelized single domain antibody, a diabody, an scfv, an scfv dimer, a dsfv, a (dsfv)2, a dsFv-dsfv', a bispecific ds diabody, an Fv, an Fab, an Fab′, an F(ab′)₂, or a domain antibody), or a pharmaceutical composition thereof further comprising a pharmaceutically acceptable carrier, comprising one or more members selected from the group consisting of: (a) CDR-L1, CDR-L2 and CDR-L3 of the variable region of 15H12/19D12 light chain C, 15H12/19D12 light chain D, 15H12/19D12 light chain E or 15H12/19D12 light chain F; or (b) CDR-H1, CDR-H2 and CDR-H3 of the variable region of 15H12/19D12 heavy chain A or 15H12/19D12 heavy chain b; or both; for example, wherein: CDR-L1 comprises the amino acid sequence: Arg Ala Ser Gln Ser Ile Gly Ser Ser Leu His (SEQ ID NO: 1); CDR-L2 comprises the amino acid sequence: Tyr Ala Ser Gln Ser Leu Ser (SEQ ID NO: 2); CDR-L3 comprises the amino acid sequence: His Gln Ser Ser Arg Leu Pro His Thr (SEQ ID NO: 3); CDR-H1 comprises the amino acid sequence: Ser Phe Ala Met His (SEQ ID NO: 4) or Gly Phe Thr Phe Ser Ser Phe Ala Met His (SEQ ID NO: 5); CDR-H2 comprises the amino acid sequence: Val Ile Asp Thr Arg Gly Ala Thr Tyr Tyr Ala Asp Ser Val Lys Gly (SEQ ID NO: 6); and CDR-H3 comprises the amino acid sequence: Leu Gly Asn Phe Tyr Tyr Gly Met Asp Val (SEQ ID NO: 7); for example, wherein the antibody or fragment comprises a light chain variable region comprising amino acids 20-128 of SEQ ID NO: 9, 11, 13 or 15 and a heavy chain variable region comprising amino acids 20-137 of SEQ ID NO: 17 or 19. In an embodiment of the invention, the antibody or fragment is in a pharmaceutical composition which further comprises a pharmaceutically acceptable carrier. In an embodiment of the invention, the antibody or fragment is linked to a constant region such as a κ light chain, γ1 heavy chain, γ2 heavy chain, γ3 heavy chain or a γ4 heavy chain. In an embodiment of the invention, the subject is administered a further chemotherapeutic agent (e.g., anti-cancer chemotherapeutic agent) or an anti-cancer therapeutic procedure (e.g., anti-cancer radiation therapy or surgical tumorectomy). In an embodiment of the invention, the further chemotherapeutic agent is one or more members selected from the group consisting of everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an EGFR TK inhibitor, an aurora kinase inhibitor, a PIK-1 modulator, a Bcl-2 inhibitor, an HDAC inhibitor, a c-MET inhibitor, a PARP inhibitor, a Cdk inhibitor, an EGFR TK inhibitor, an IGFR-TK inhibitor, an anti-HGF antibody, a PI3 kinase inhibitors, an AKT inhibitor, a JAK/STAT inhibitor, a checkpoint-1 or 2 inhibitor, a focal adhesion kinase inhibitor, a Map kinase kinase (mek) inhibitor, a VEGF trap antibody, pemetrexed, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR, KRX-0402, lucanthone, LY 317615, neuradiab, vitespan, Rta 744, Sdx 102, talampanel, atrasentan, Xr 311, romidepsin, ADS-100380, sunitinib, 5-fluorouracil, leucovorin,

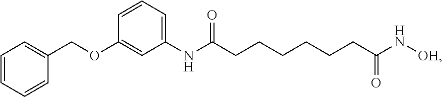

### CG-781, CG-1521,

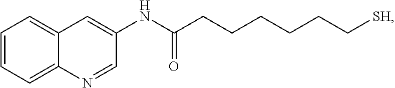

SB-556629, chlamydocin, JNJ-16241199,

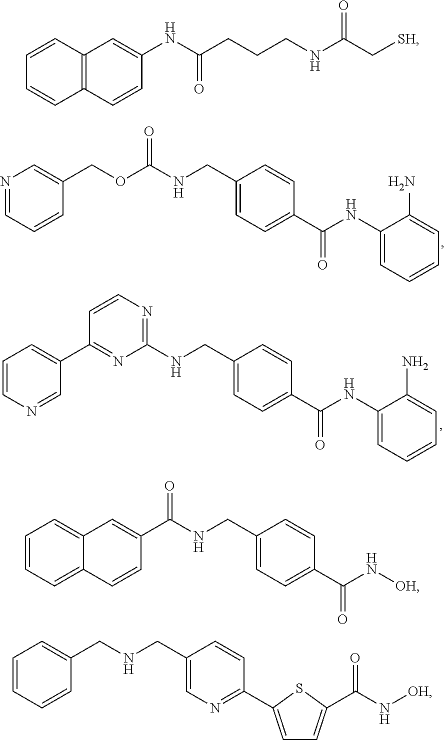

vorinostat, etoposide, gemcitabine, doxorubicin, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin, PEG-labeled irinotecan, tamoxifen, toremifene citrate, anastrazole, exemestane, letrozole, DES (diethylstilbestrol), estradiol, estrogen, conjugated estrogen, bevacizumab, IMC-1C11, CHIR-258,

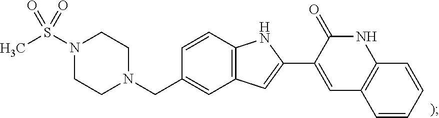

3-[5-(methylsulfonylpiperadinemethyl)indoly]-quinolone, vatalanib, AG-013736, AVE-0005, the acetate salt of [D-Ser(Bu t) 6, Azgly 10] (pyro-Glu-His-Trp-Ser-Tyr-D-Ser(Bu t)-Leu-Arg-Pro-Azgly-NH₂ acetate [C₅₉H₈₄N₁₈O₁₄.(C₂H₄O₂)ₓ where x=1 to 2.4], goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, lonafarnib,

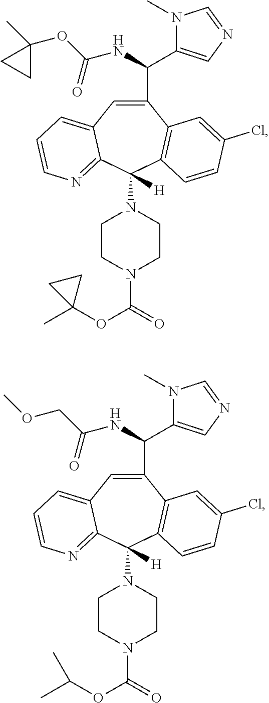

BMS-214662, tipifarnib; amifostine, NVP-LAQ824, suberoyl analide hydroxamic acid, valproic acid, trichostatin A, FK-228, SU11248, sorafenib, KRN951, aminoglutethimide, amsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mercaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, cremophor-free paclitaxel, docetaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, topotecan, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa and darbepoetin alfa

### BRIEF DESCRIPTION OF THE FIGURES

FIG. 1. Colorectal HT29 model sequencing study design. Ir=irinotecan; anti-IGF1R=LCF/HCA antibody.

FIG. 2. Colorectal HT29 model sequencing study results (0.1 mg anti-IGF1R). Irinotecan was adminsterd on the days indicated with arrows (days 8, 12 and 15). Anti-IGF1R was administered on the days indicated with the arrows (days 8, 12 or 15 simultaneous with irinotecan; or days 18, 22 and 26 either alone or following irinotecan). Ir=irinotecan; anti-IGF1R=LCF/HCA antibody. Percentages indicate percentage of tumor growth inhibition relative to the control (IgG1+vehicle, black diamonds).

FIG. 3. Colorectal HT29 model sequencing study results (0.5 mg anti-IGF1R). Irinotecan was adminsterd on the days indicated with arrows (days 8, 12 and 15). Anti-IGF1R was administered on the days indicated with the arrows (days 8, 12 or 15 simultaneous with irinotecan; or days 18, 22 and 26 either alone or following irinotecan). Ir=irinotecan; anti-IGF1R=LCF/HCA antibody. Percentages indicate percentage of tumor growth inhibition relative to the control (IgG1+vehicle, black diamonds).

FIG. 4. Colorectal HT29 model sequencing study results. Combination of the data set forth in FIGS. 2 and 3 into a single plot for ease of comparison. Ir=irinotecan; anti-IGF1R=LCF/HCA antibody. Percentages indicate percentage of tumor growth inhibition relative to the control (IgG1+vehicle, black diamonds).

FIG. 5. Osteosarcoma SJSA-1 model sequencing study design. CX=cyclophosphamide; Ab.=LCF/HCA anti-IGF1R antibody.

FIG. 6. Osteosarcoma SJSA-1 model sequencing study results.

FIG. 7. Osteosarcoma SJSA-1 model sequencing study result, end of study tumor volume and growth inhibition.

FIG. 8. Osteosarcoma SJSA-1 model sequencing study result, follow-up. Tumor volume followed after treatment cessation after day 38.

### DETAILED DESCRIPTION OF THE INVENTION

The present invention provides, in part, methods for treating or preventing a medical condition mediated by elevated expression or activity of IGF1R and/or overexpression of IGF-I and/or IGF-II by first administering a cytotoxic agent (e.g., irinotecan or cyclophosphamide), then an IGF1R inhibitor. Such sequential administration of such chemotherapeutic agents has proven to have efficacy far superior to that of non-sequential administration (e.g., co-administration). Sequential administration includes, in an embodiment of the invention, administering the cytotoxic agent prior to administration of the IGF1R inhibitor; for example, 1 or 2 or 3 or 4 or 5 or 6 or 7 days prior to administration of the IGF1R inhibitor. For example, the present invention includes embodiments wherein a subject is administered the cytotoxic agent, then the IGF1R inhibitor, then the cytotoxic agent, then the IGF1R inhibitor, etc, (for example, 1, 2, 3 or 4 or more cycles of cytotoxic agent, then IGF1R inhibitor) as part of a continuous treatment cycle regimen wherein each combined treatment, of cytotoxic agent, then IGF1R inhibitor, is considered one treatment cycle.

The present invention also includes embodiments wherein the patient is given one or more (e.g., 1, 2, 3, 4, 5, 6 or 7), treatments of cytotoxic agent, followed by one or more (e.g., 1, 2, 3, 4, 5, 6 or 7), treatments of the IGF1R inhibitor. In such embodiments of the present invention, each episode of one or more treatments with the cytotoxic agent, followed by an episode or one or more treatments with the IGF1R inhibitor, would be considered one treatment cycle. The present invention also encompasses methods comprising several of these treatment cycles (e.g., 1, 2, 3, 4, 5, 6, 7, 8, 9, 10).

A “hyperproliferative disease” is a disorder characterized by abnormal proliferation of cells, and generically includes, e.g., benign and malignant tumors of all organ systems (e.g., colorectal cancer or osteosarcoma). A “tumor” is a neoplasm, and includes both solid and non-solid tumors (such as hematologic malignancies).

### Cytotoxic Agents

The present invention provides methods for treating or preventing hyperproliferative disorders by administering an anti-cancer cytotoxic chemotherapeutic agent (e.g., irinotecan or cyclophosphamide), then an IGF1R inhibitor.

In an embodiment of the invention, an anti-cancer cytotoxic chemotherapeutic agent is an agent which is toxic to cells, in particular an agent which is more cytotoxic to cancer cells or cells in a hyperproliferative state than to cells which divide normally and/or which are not cancerous. For example, such agents include those which induce apoptosis, inhibit nucleic acid (e.g., DNA) synthesis, stabilize microtubule polymerization, inhibit topoisomerase (e.g., topoisomerase I), interfere with microtubule assembly, interfere with signal transduction, inhibit angiogenesis, and/or inhibit cellular division, in particular of hyperproliferative and/or cancerous cells.

In an embodiment of the invention, irinotecan is characterized by the following structural formula.

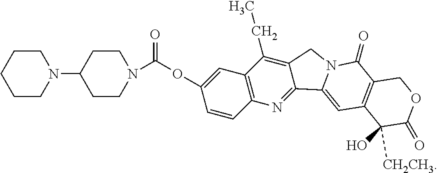

The term includes, for example, salts thereof such as, for example, the monohydrochloride, trihydrate thereof. The term also includes PEGylated irinotecan (PEG-irinotecan), for example, NKTR-102.

In an embodiment of the invention, cyclophosphamide is characterized by the following structural formula:

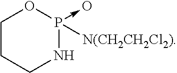

Cyclophosphamide is sold commercially as Cytoxan or as Neosar. The term cyclophosphamide includes hydrates thereof (e.g., monohydrate).

### Antibodies and Antigen-Binding Fragments Thereof

In an embodiment of the invention, subjects are administered an anti-IGF1R antibody or antigen-binding fragment thereof, e.g., that specifically binds to IGF1R, which comprises light chain CDRs or heavy chain CDRs or both, for example, as set forth below, following administration of irinotecan or cyclophosphamide.

15H12/19D12 light chain immunoglobulin CDRs

| | |
|---|---|
| CDR-L1: RASQSIGSSLH | (SEQ ID NO: 1) |
| CDR-L2: YASQSLS; | (SEQ ID NO: 2) |
| CDR-L3: HQSSRLPHT; | (SEQ ID NO: 3) |

for example, all three light chain immunoglobulin CDRs;
and/or

15H12/19D12 heavy chain immunoglobulin CDRs

| | | |
|---|---|---|
| CDR-H1: | SFAMH; | (SEQ ID NO: 4) |

or

| | | |
|---|---|---|
| | GFTFSSFAMH; | (SEQ ID NO: 5) |
| CDR-H2: | VIDTRGATYYADSVKG; | (SEQ ID NO: 6) |
| CDR-H3: | LGNFYYGMDV; | (SEQ ID NO: 7) |

for example, all three heavy chain immunoglobulin CDRs.

In an embodiment of the invention, the antibody comprises any combination of the following light and heavy chain immunoglobulin chains (e.g., mature fragments thereof) or antigen-binding fragments thereof or CDRs thereof, e.g., CDRs as defined by Kabat et al., Sequences of Proteins of Immunological Interest, 5th Ed. Public Health Service, National Institutes of Health, Bethesda, Md. (1991); and/or, Chothia and Lesk, J. Mol. Biol. 196:901-917 (1987). In an embodiment of the invention, the anti-IGF1R antibody or antigen-binding fragment thereof binds to the same IGF1R epitope as any of those that are set forth herein (but which is a non-identical antibody or fragment); or competes for binding to an IGF1R epitope with any of those set forth herein (but which is a non-identical antibody or fragment). Signal sequences are underscored with dashed lines and CDR sequences are underscored by solid lines. In an embodiment of the invention, mature variable region fragments lack the signal sequences.

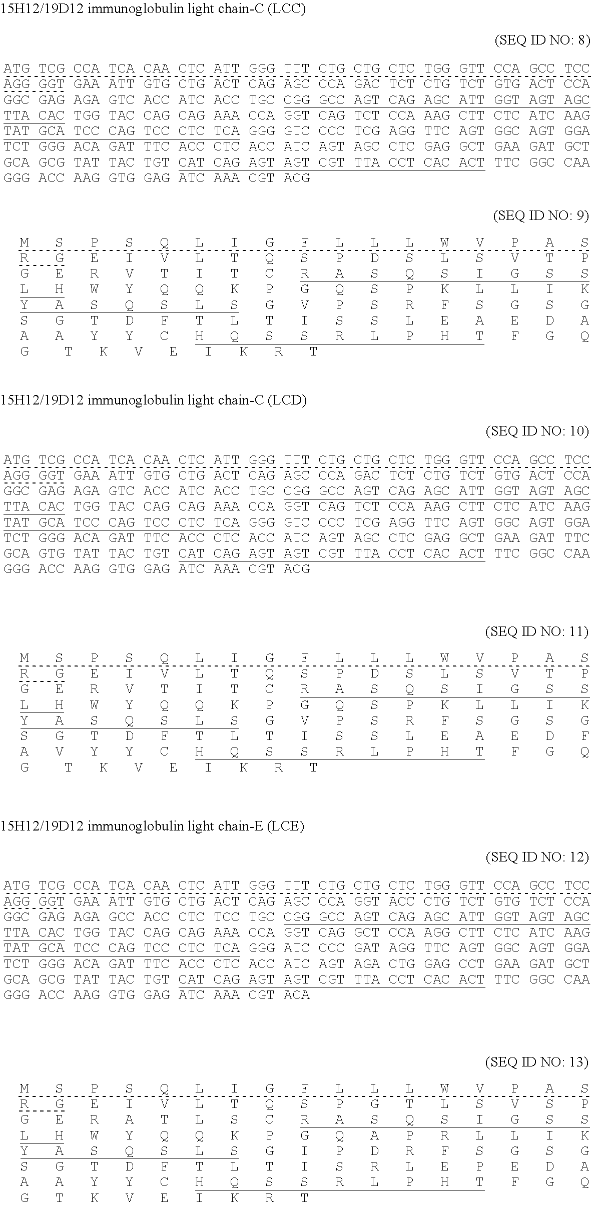

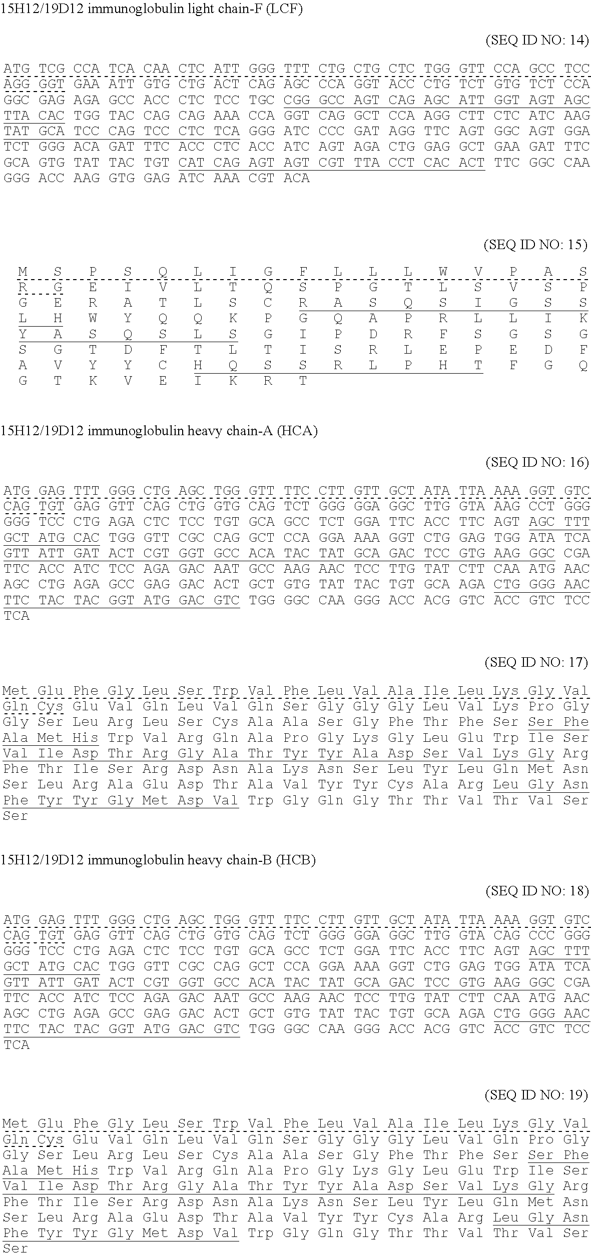

See U.S. Pat. No. 7,217,796; any anti-IGF1R or antigen-binding fragment thereof in the patent can be used in a method of the present invention.

In an embodiment of the present invention, the anti-IGF1R antibody light chain and/or heavy chain is encoded by any plasmid selected from the group consisting of:

(i) CMV promoter—15H12/19D12 HCA (γ4)—Deposit name: “15H12/19D12 HCA (γ4)”; ATCC accession No.: PTA-5214;
(ii) CMV promoter—15H12/19D12 HCB γ4)—Deposit name: “15H12/19D12 HCB (γ4)”; ATCC accession No.: PTA-52 15;
(iii) CMV promoter—15H12/19D12 HCA (γ1)—Deposit name: “15H12/19D12 HCA (γ1)”; ATCC accession No.: PTA-5216;
(iv) CMV promoter—15H12/19D12 LCC (κ)—Deposit name: “15H12/19D12 LCC (κ)”; ATCC accession No.: PTA-5217;
(v) CMV promoter—15H12/19D12 LCD (κ)—Deposit name: “15H12/19D12 LCD (κ)”; ATCC accession No.: PTA-5218;
(vi) CMV promoter—15H12119D12 LCE (κ)—Deposit name: “15H12/19D12 LCE (κ)”; ATCC accession No.: PTA-5219; and
(vii) CMV promoter—15H12119D12 LCF (κ)—Deposit name: “15H12/19D12 LCF (κ)”, ATCC accession No.: PTA-5220;

The above-identified plasmids were deposited, under the Budapest Treaty, on May 21, 2003, with the American Type Culture Collection (ATCC); University Boulevard; Manassas, Va. 20110 2209. All restrictions on the accessibility of the deposited plasmids to the public have been irrevocably removed by the applicant upon the granting of U.S. Pat. No. 7,217,796.

In an embodiment of the invention, the antibody is an LCC/HCA (i.e., comprising light chain C and heavy chain A), LCD/HCB (i.e., comprising light chain D and heavy chain B) or LCF/HCA (i.e., comprising light chain F and heavy chain A).

In an embodiment of the invention, the anti-IGF1R antibody or antigen-binding fragment thereof comprises the mature heavy chain immunoglobulin variable region:

(SEQ ID NO: 20)
vqllesggglvqpggslrlsctasgftfssyamnwvrqapgkglewvsai
sgsggttfyadsvkgrftisrdnsrttylqmnslraedtavyycakdlgw
sdsyyyyygmdvwgqgttvtvss;

or one or more CDRs (e.g., 3) therefrom.

In an embodiment of the invention, the anti-IGF1R antibody or antigen-binding fragment thereof comprises the mature light chain immunoglobulin variable region:

(SEQ ID NO: 21)
diqmtqfpsslsasvgdrytitcrasqgirndlgwyqqkpgkapkrliya
asrlhrgvpsrfsgsgsgteftltisslqpedfatyyclqhnsypcsfgq
gtkleik;

(or one or more CDRs (e.g., 3) therefrom.

The present invention includes methods for using anti-IGF1R antibodies and antigen-binding fragments thereof. Thus, the invention includes methods for using monoclonal antibodies, camelized single domain antibodies, polyclonal antibodies, bispecific antibodies, chimeric antibodies, recombinant antibodies, anti-idiotypic antibodies, humanized antibodies, bispecific antibodies, diabodies, single chain antibodies, disulfide Fvs (dsfv), Fvs, Fabs, Fab′s, F(ab′)₂s and domain antibodies (the meanings of which are well known in the art). Thus, the term “antibody” and the like covers, but is not limited to, monoclonal antibodies, polyclonal antibodies, recombinant antibodies, multispecific antibodies (e.g., bispecific antibodies) (the meanings of which are well known in the art). The term “antigen-binding fragment” and the like of an antibody (of the “parental antibody”) encompasses a fragment or a derivative of an antibody, typically including at least a portion of the antigen-binding or variable region (e.g., one or more CDRs) of the parental antibody, that retains at least some of the binding specificity of the parental antibody. Examples of antibody antigen-binding fragments include, but are not limited to, Fab, Fab′, F(ab′)₂, and Fv fragments; diabodies; single-chain antibody molecules, e.g., sc-Fv; and multispecific antibodies formed from antibody fragments (the meanings of which are well known in the art). In an embodiment of the invention, a binding fragment or derivative retains at least 10% of its IGF1R binding activity when that activity is expressed on a molar basis. In an embodiment of the invention, a binding fragment or derivative retains at least 20%, 50%, 70%, 80%, 90%, 95% or 100% or more of the IGF1R binding affinity as the parental antibody. It is also intended that an antigen-binding fragment can include conservative amino acid substitutions (referred to as “conservative variants” of the antibody) that do not substantially alter its biologic activity.

In an embodiment of the invention, “Fab” refers to a fragment including a single light chain (both variable and constant regions) bound to the variable region and first constant region of a single heavy chain by a disulfide bond. Fab fragments may be produced by, for example, papain digestion of an IgG antibody.

In an embodiment of the invention, “Fab′” refers to a Fab fragment that includes a portion of the hinge region.

In an embodiment of the invention, “F(ab)₂” refers to a dimer of Fab′. F(ab)₂ fragments which may be produced by enzymatic cleavage of an IgG by, for example, pepsin.

In an embodiment of the invention, an “Fc” region comprises two heavy chain fragments comprising the C_{H}1 and C_{H}2 domains of an antibody. The two heavy chain fragments are held together by two or more disulfide bonds and by hydrophobic interactions of the C_{H}3 domains.

In an embodiment of the invention, a “nanobody” is the VHH domain of a heavy-chain antibody. Such heavy chain antibodies contain a single variable domain (VHH) and two constant domains (CH2 and CH3).

In an embodiment of the invention, a “disulfide stabilized Fv fragment” or “dsFv” comprises molecules having a variable heavy chain (V_{H}) and a variable light chain (V_{L}) which are linked by a disulfide bridge.

In an embodiment of the invention, an “Fv region” comprises the variable regions from both the heavy and light chains, but lacks the constant regions.

In an embodiment of the invention, the term “single-chain Fv” or “scFv” antibody comprises antibody fragments comprising the V_{H} and V_{L} domains of an antibody, wherein these domains are present in a single polypeptide chain. Generally, the polypeptide further comprises a polypeptide linker between the V_{H} and V_{L} domains which enables the V_{H} and V_{L} chains to pair and form a binding site (e.g., 5-12 residues long). For a review of scFv, see Pluckthun (1994) THE PHARMACOLOGY OF MONOCLONAL ANTIBODIES, vol. 113, Rosenburg and Moore eds. Springer-Verlag, New York, pp. 269-315. See also, International Patent Application Publication No. WO 88/01649 and U.S. Pat. Nos. 4,946,778 and 5,260,203.

In an embodiment of the invention, a “domain antibody” comprises an immunologically functional immunoglobulin fragment containing only the variable region of a heavy chain or the variable region of a light chain. In some instances, two or more V_{H} regions are covalently joined with a peptide linker to create a bivalent domain antibody. The two V_{H} regions of a bivalent domain antibody may target the same or different antigens.

In an embodiment of the invention, a “bivalent” or “bispecific” antibody comprises two antigen-binding sites. In some instances, the two binding sites have the same antigen specificities. However, bivalent antibodies may be bispecific. For example, the present invention comprises scfv dimers and dsfv dimers, each of which scfv and dsfv moieties may have a common or different antigen binding specificity.

In an embodiment of the invention, a (dsfv)₂ comprises three peptide chains: two V_{H} moieties linked by a peptide linker and bound by disulfide bridges to two V_{L} moieties.

In an embodiment of the invention, a “bispecific ds diabody” comprises a VH₁-VL₂ (tethered by a peptide linker) linked, by a disulfide bridge between the VH₁ and VL₁, to a VL₁-VH₂ moiety (also tethered by a peptide linker).

In an embodiment of the invention, a “bispecific dsfv-dsfv′” also comprises three peptide chains: a VH₁-VH₂ moiety wherein the heavy chains are linked by a peptide linker (e.g., a long flexible linker) and are bound to VL₁ and VL₂ moieties, respectively, by disulfide bridges; wherein each disulfide paired heavy and light chain has a different antigen specificity.

In an embodiment of the invention, an “scfv dimer” (a bivalent diabody) comprises a V_{H}-V_{L} moiety wherein the heavy and light chains are bound to by a peptide linker and dimerized with another such moiety such that V_{H}s of one chain coordinate with the V_{L}s of another chain and form two identical binding sites.

In an embodiment of the invention a “bispecific diabody” comprises VH₁-VL₂ moiety (linked by a peptide linker) associated with a VL₁-VH₂ (linked by a peptide linker), wherein the VH₁ and VL₁ coordinate and the VH₂ and VL₂ coordinate and each coordinated set has diverse antigen specificities.

In an embodiment of the invention, the term “monoclonal antibody” comprises an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic epitope. In contrast, conventional (polyclonal) antibody preparations typically include a multitude of antibodies directed against (or specific for) different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made recombinantly or by the hybridoma method first described by Kohler et al. (1975) Nature 256: 495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352: 624-628 and Marks et al. (1991) J. Mol. Biol. 222: 581-597, for example. See also Presta (2005) J. Allergy Clin. Immunol. 116:731.

In an embodiment of the invention, “chimeric” antibodies (immunoglobulins) include a portion of the heavy and/or light chain that is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., (1984) Proc. Natl. Acad. Sci. USA 81: 6851-6855). For example, variable domains are obtained from an antibody from an experimental animal (the “parental antibody”), such as a mouse, and the constant domain sequences are obtained from human antibodies, so that the resulting chimeric antibody will be less likely to elicit an adverse immune response in a human subject than the parental mouse antibody.

In an embodiment of the invention, a recombinant antibody or antigen-binding fragment thereof of the invention is an antibody which is produced recombinantly, e.g., expressed from a polynucleotide which has been introduced into an organism (e.g., a plasmid containing a polynucleotide encoding the antibody or fragment transformed into a bacterial cell (e.g., E. coli) or a mammalian cell (e.g., CHO cell)), followed by isolation of the antibody or fragment from the organism.

The present invention also includes camelized single domain antibodies, for example, comprising one or more (e.g., 3) of the anti-IGF1R CDRs set forth herein. See, e.g., Muyldermans et al. (2001) Trends Biochem. Sci. 26:230; Reichmann et al. (1999) J. Immunol. Methods 231:25; WO 94/04678; WO 94/25591; U.S. Pat. No. 6,005,079, which are hereby incorporated by reference in their entireties). Camelidae (camels, dromedaries and llamas) comprise IgG antibodies in which are devoid of light chains and therefore called ‘heavy-chain’ IgGs or HCAb (for heavy-chain antibody). HCAbs typically have a molecular weight of ˜95 kDa since they consist only of the heavy-chain variable domains. Although the HCAbs are devoid of light chains, they have an authentic antigen-binding repertoire (Hamers-Casterman et al., Nature (1993) 363:446-448; Nguyen et al., Adv. Immunol. (2001) 79:261-296; Nguyen et al., Immunogenetics. (2002) 54:39-47). In one embodiment, the present invention provides single domain antibodies comprising two V_{H} domains with modifications such that single domain antibodies are formed.

In an embodiment of the invention, the term “diabodies” includes small antibody fragments with two antigen-binding sites, which fragments comprise a heavy chain variable domain (V_{H}) connected to a light chain variable domain (V_{L}) in the same polypeptide chain (V_{H}-V_{L} or V_{L}-V_{H}). By using a linker that is too short to allow pairing between the two domains on the same chain, the domains are forced to pair with the complementary domains of another chain and create two antigen-binding sites. Diabodies are described more fully in, e.g., EP 404,097; WO 93/11161; and Holliger et al. (1993) Proc. Natl. Acad. Sci. USA 90: 6444-6448. For a review of engineered antibody variants generally see Holliger and Hudson (2005) Nat. Biotechnol. 23:1126-1136.

In an embodiment of the invention, the term “humanized antibody” comprises forms of antibodies that contain sequences from both human and non-human (e.g., murine or rat) antibodies. In general, the humanized antibody will comprise substantially all of at least one, and typically two, variable domains, in which all or substantially all of the hypervariable loops correspond to those of a non-human immunoglobulin, and all or substantially all of the framework (FR) regions are those of a human immunoglobulin sequence. The humanized antibody may optionally comprise at least a portion of a human immunoglobulin constant region (Fc).

For example, the present invention comprises any humanized antibody comprising the CDRs of 15H12/19D12, e.g., wherein identical CDRs were originally isolated from a non-human species antibody and incorporated into a human antibody framework.

The antibodies of the present invention also include antibodies with modified (or blocked) Fc regions to provide altered effector functions. See, e.g., U.S. Pat. No. 5,624,821; WO2003/086310; WO2005/120571; WO2006/0057702. Such modifications can be used to enhance or suppress various reactions of the immune system, with possible beneficial effects in diagnosis and therapy. Alterations of the Fc region include amino acid changes (substitutions, deletions and insertions), glycosylation or deglycosylation, and adding multiple Fc. Changes to the Fc can also alter the half-life of antibodies in therapeutic antibodies, enabling less frequent dosing and thus increased convenience and decreased use of material. See Presta (2005) J. Allergy Clin. Immunol. 116:731 at 734-35.

The anti-IGF1R antibodies and antigen-binding fragments thereof of the invention are, in an embodiment of the invention, conjugated to a chemical moiety. The chemical moiety may be, inter alia, a polymer, a radionuclide or a cytotoxic factor. In an embodiment of the invention, the chemical moiety is a polymer which increases the half-life of the antibody or fragment in the body of a subject to whom it is administered. Polymers include, but are not limited to, polyethylene glycol (PEG) (e.g., PEG with a molecular weight of 2 kDa, 5 kDa, 10 kDa, 12 kDa, 20 kDa, 30 kDa or 40 kDa), dextran and monomethoxypolyethylene glycol (mPEG). Lee, et al., (1999) (Bioconj. Chem. 10:973-981) discloses PEG conjugated single-chain antibodies. Wen, et al., (2001) (Bioconj. Chem. 12:545-553) disclose conjugating antibodies with PEG which is attached to a radiometal chelator (diethylenetriaminpentaacetic acid (DTPA)).

The antibodies and antigen-binding fragments of the invention are, in an embodiment of the invention, conjugated with labels such as ^{99m}Tc, ⁹⁹Tc, ⁹⁰Y, ¹¹¹In, ³²P, ¹⁴C, ¹²⁵I, ³H, ¹³¹I, ¹²³I, ¹¹C, ¹⁵O, ¹³N, ¹⁸F, ³⁵S, ⁵¹Cr, ⁵⁷To, ²²⁶Ra, ⁶⁰Co, ⁵⁹Fe, ⁵⁷Se, ¹⁵²Eu, ⁶⁷CU, ²¹⁷Ci, ²¹¹At, ²¹²Pb, ⁴⁷Sc, ¹⁰⁷Pd, ²³⁴Th, ⁴⁰K, ¹⁵⁷Gd, ⁵⁵Mn, ⁵²Tr and ⁵⁶Fe.

The antibodies and antigen-binding fragments of the invention may also be conjugated with fluorescent or chemilluminescent labels, including fluorophores such as rare earth chelates, fluorescein and its derivatives, rhodamine and its derivatives, isothiocyanate, phycoerythrin, phycocyanin, allophycocyanin, o-phthaladehyde, fluorescamine, ¹⁵²Eu, dansyl, umbelliferone, luciferin, luminal label, isoluminal label, an aromatic acridinium ester label, an imidazole label, an acridimium salt label, an oxalate ester label, an aequorin label, 2,3-dihydrophthalazinediones, biotin, avidin, peroxidase such as horseradish peroxidase, alkaline phosphatase (e.g., calf, shrimp or bacterial), spin labels and stable free radicals.

The antibodies and antigen-binding fragments of the invention may also be conjugated to a cytotoxic factor such as diptheria toxin, Pseudomonas aeruginosa exotoxin A chain, ricin A chain, abrin A chain, modeccin A chain, alpha-sarcin, Aleurites fordii proteins and compounds (e.g., fatty acids), dianthin proteins, Phytoiacca americana proteins PAPI, PAPII, and PAP-S, momordica charantia inhibitor, curcin, crotin, saponaria officinalis inhibitor, mitogellin, restrictocin, phenomycin, and enomycin.

Any method known in the art for conjugating the antibodies and antigen-binding fragments of the invention to the various moieties may be employed, including those methods described by Hunter, et al., (1962) Nature 144:945; David, et al., (1974) Biochemistry 13:1014; Pain, et al., (1981) J. Immunol. Meth. 40:219; and Nygren, J., (1982) Histochem. and Cytochem. 30:407. Methods for conjugating antibodies are conventional and very well known in the art.

### Further Chemotherapeutic Agents

The present invention comprises methods for treating hyperproliferative disorders by administering irinotecan or cyclophosphamide, then an IGF1R antagonist. In addition to this regimen, the patient may be administered a further chemotherapeutic agent, e.g., in association with administration of the cytotoxic agent and/or IGF1R antagonist. In an embodiment of the invention, the further chemotherapeutic agent is an anti-cancer agent. In an embodiment of the invention, the further chemotherapeutic agent is one or more members selected from the group consisting of: everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an EGFR TK inhibitor, an aurora kinase inhibitor, a PIK-1 modulator, a Bcl-2 inhibitor, an HDAC inhibitor, a c-MET inhibitor, a PARP inhibitor, a Cdk inhibitor, an EGFR TK inhibitor, an IGFR-TK inhibitor, an anti-HGF antibody, a PI3 kinase inhibitors, an AKT inhibitor, a JAK/STAT inhibitor, a checkpoint-1 or 2 inhibitor, a focal adhesion kinase inhibitor, a Map kinase kinase (mek) inhibitor, a VEGF trap antibody, pemetrexed, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR, KRX-0402, lucanthone, LY 317615, neuradiab, vitespan, Rta 744, Sdx 102, talampanel, atrasentan, Xr 311, romidepsin, ADS-100380,

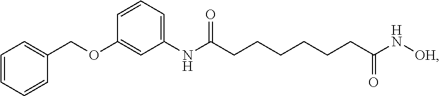

### CG-781, CG-1521,

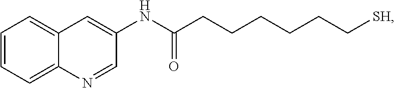

SB-556629, chlamydocin, JNJ-16241199,

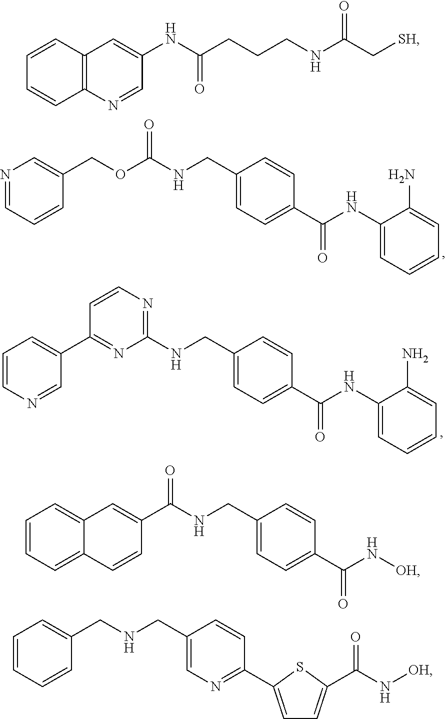

vorinostat, etoposide, gemcitabine, doxorubicin, liposomal doxorubicin, 5′-deoxy-5-fluorouridine, vincristine, temozolomide (optionally further including irinotecan; e.g., in a method to treat glioblastoma multiforme, for example, comprising administering the antibody or fragment, temozolomide and radiation therapy; or administering the antibody or fragment, temozolomide and irinotecan), ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin, tamoxifen, toremifene citrate, anastrazole, exemestane, letrozole, DES (diethylstilbestrol), estradiol, estrogen, conjugated estrogen, bevacizumab, IMC-1C11, CHIR-258,

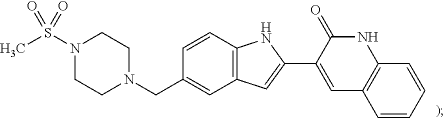

3-[5-(methylsulfonylpiperadinemethyl)indolyl]-quinolone, vatalanib, AG-013736, AVE-0005, the acetate salt of [D-Ser(Bu t) 6, Azgly 10] (pyro-Glu-His-Trp-Ser-Tyr-D-Ser(Bu t)-Leu-Arg-Pro-Azgly-NH₂ acetate [C₅₉H₈₄N₁₈O₁₄.(C₂H₄O₂)ₓ where x=1 to 2.4], goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, lonafarnib,

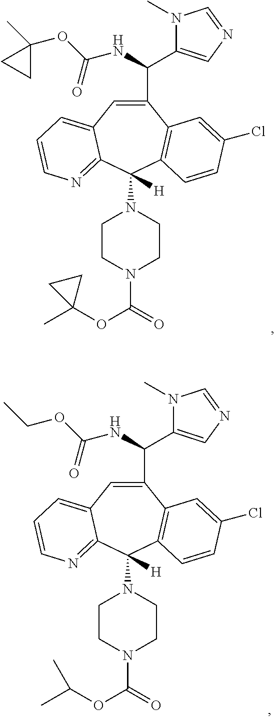

BMS-214662, tipifarnib; amifostine, NVP-LAQ824, suberoyl analide hydroxamic acid, valproic acid, trichostatin A, FK-228, SU11248, sorafenib, KRN951, aminoglutethimide, amsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mercaptopurine, deoxycoformycin, calcitriol, valrubicin, mithramycin, vinblastine, vinorelbine, topotecan, razoxin, marimastat, COL-3 (metastat), neovastat, BMS-275291

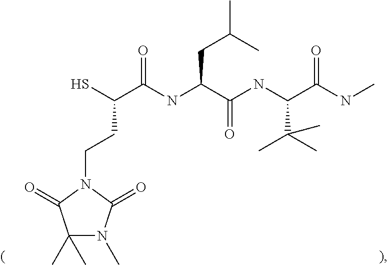

squalamine, endostatin, SU5416 (semaxinib), SU6668 ([(Z)-3-[2,4-dimethyl-5-(2-oxo-1,2-dihydro-indol-3-ylidenemethyl)-1H-pyrrol-3-yl]-propionic acid), EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, cremophor-free paclitaxel, docetaxel, epithilone B, BMS-247550 (ixabepilone), BMS-310705

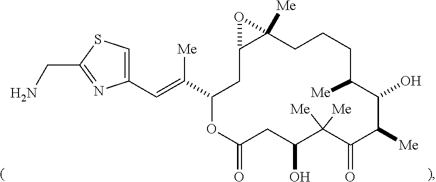

droloxifene, 4-hydroxytamoxifen, pipendoxifene, ERA-923 (2-(4-Hydroxy-phenyl)-3-methyl-1-[4-(2-piperidin-1-yl-ethoxy)-benzyl]-1H-indol-5-ol hydrochloride), arzoxifene, fulvestrant, acolbifene, lasofoxifene, idoxifene, TSE-424 (bazedoxifene acetate), HMR-3339 (4-chloro-11b-[4-(2-[diethylamino]ethoxy)phenyl]-estra-1,3,5(10)-triene-3,17b-diol), ZK186619, topotecan, PTK787/ZK 222584, VX-745

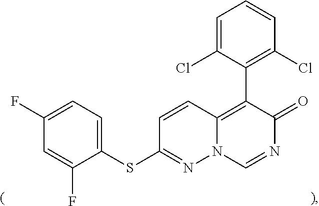

PD184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, filgrastim, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase and strontium 89.

In an embodiment of the invention, the further chemotherapeutic agent is an orally administrable formulation comprising etoposide (e.g., a liquid filled, soft gelatin capsule comprising 50 mg of etoposide in a vehicle consisting of citric acid, glycerin, purified water, and polyethylene glycol 400, wherein the soft gelatin capsules contain gelatin, glycerin, sorbitol, purified water, and parabens (ethyl and propyl) with the following dye system: iron oxide (red) and titanium dioxide), e.g., for use in a method for treating ovarian cancer.

In certain embodiments, the further chemotherapeutic agent is a PD-1 inhibitor, a chk1 inhibitor, a ras inhibitor (e.g., a farnesyl protein transferase inhibitor), a PTEN inhibitor, a hormone receptor antagonist (e.g., estrogen receptor alpha or beta or progesterone receptor), a transcription factor inhibitor, pertuzumab, altretamine, a nitrosourea (e.g., semustine, ethylnitrosourea (ENU) or Streptozotocin), or FOLFIRI regimen (folinic acid, fluorouracil (5-FU) and irinotecan; e.g., irinotecan (180 mg/m² IV over 90 minutes) concurrently with folinic acid (400 mg/m² [or 2×250 mg/m²] IV over 120 minutes), followed by fluorouracil (400-500 mg/m² IV bolus) then fluorouracil (2400-3000 mg/m² intravenous infusion over 46 hours);e.g., in a method for treating colorectal cancer).

In certain embodiments, the further chemotherapeutic agent is one or more compounds selected from the group consisting of:

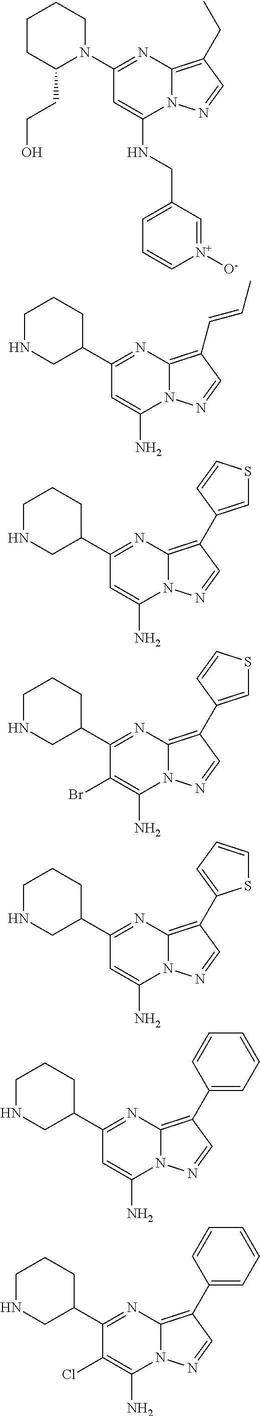

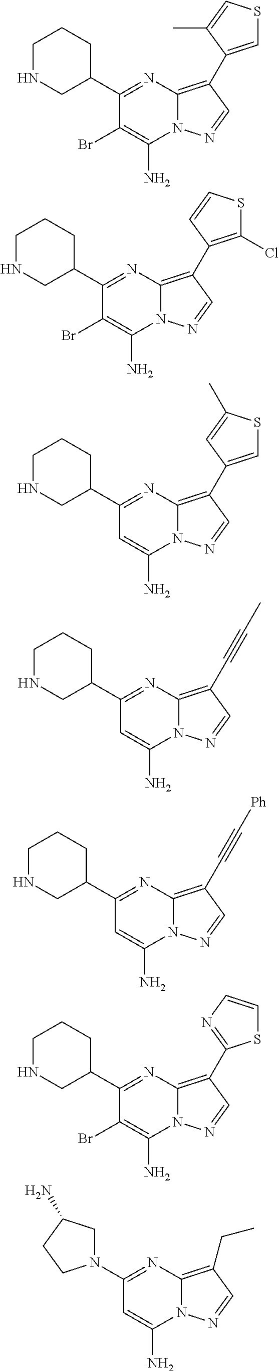

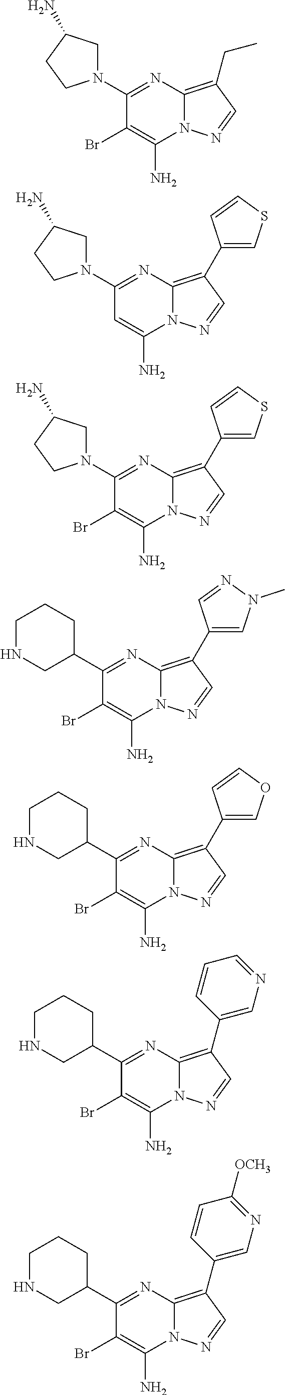

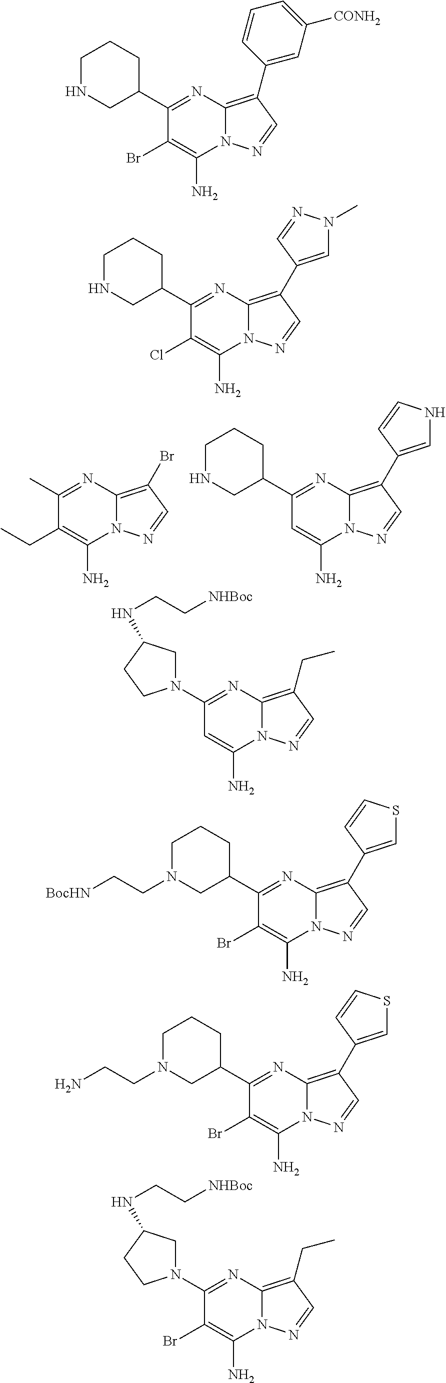

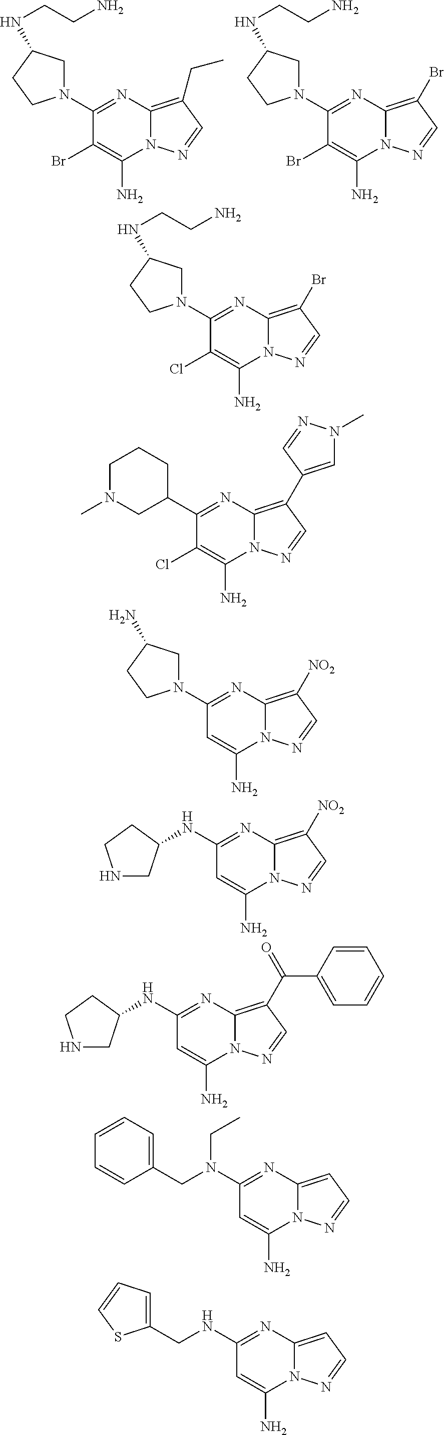

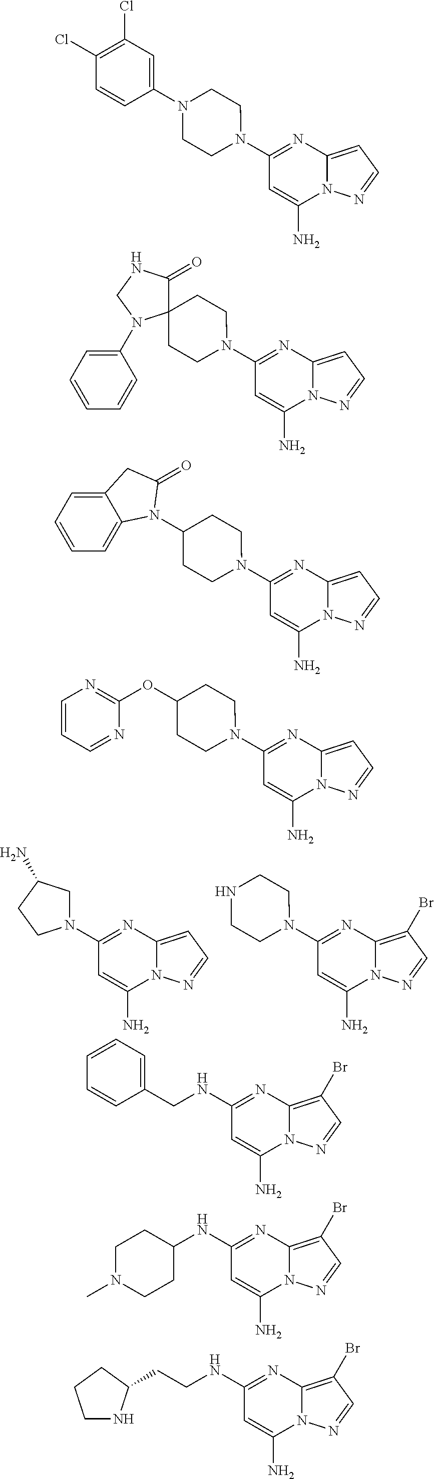

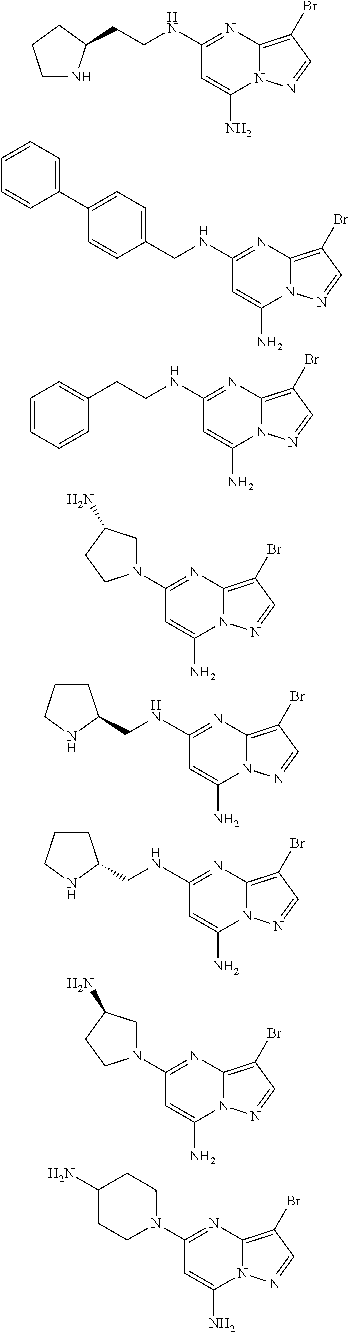

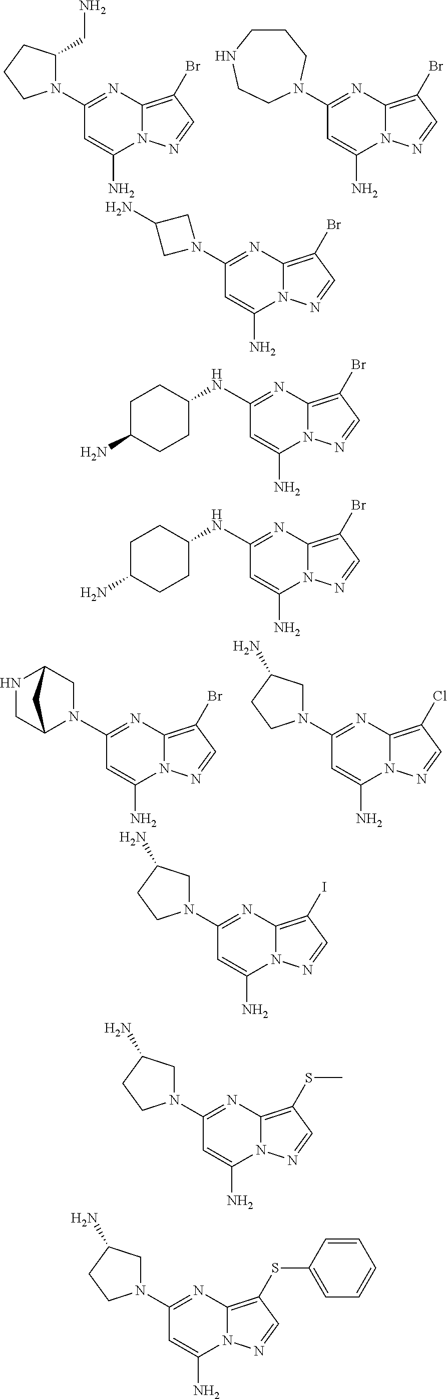

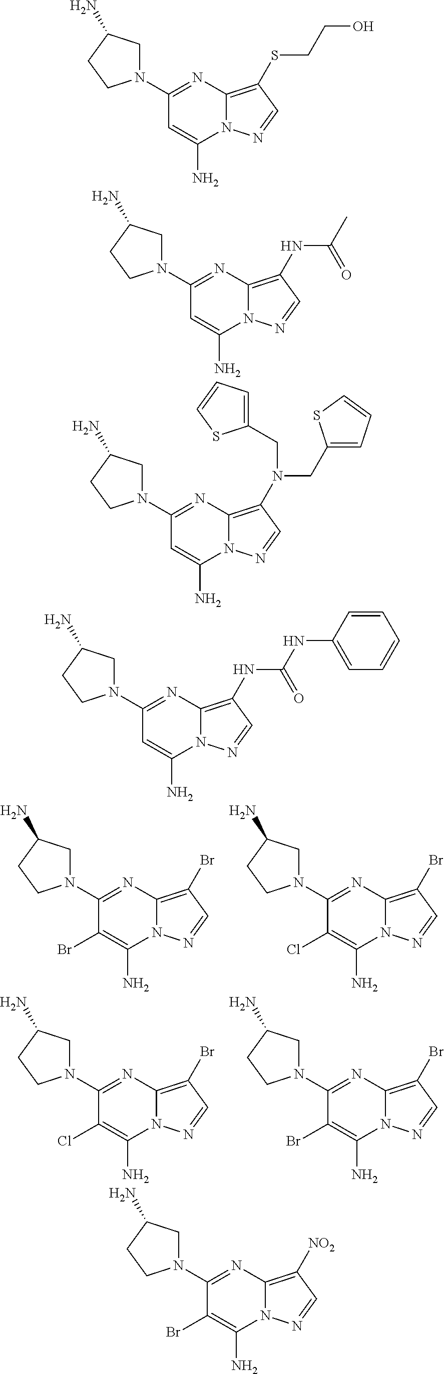

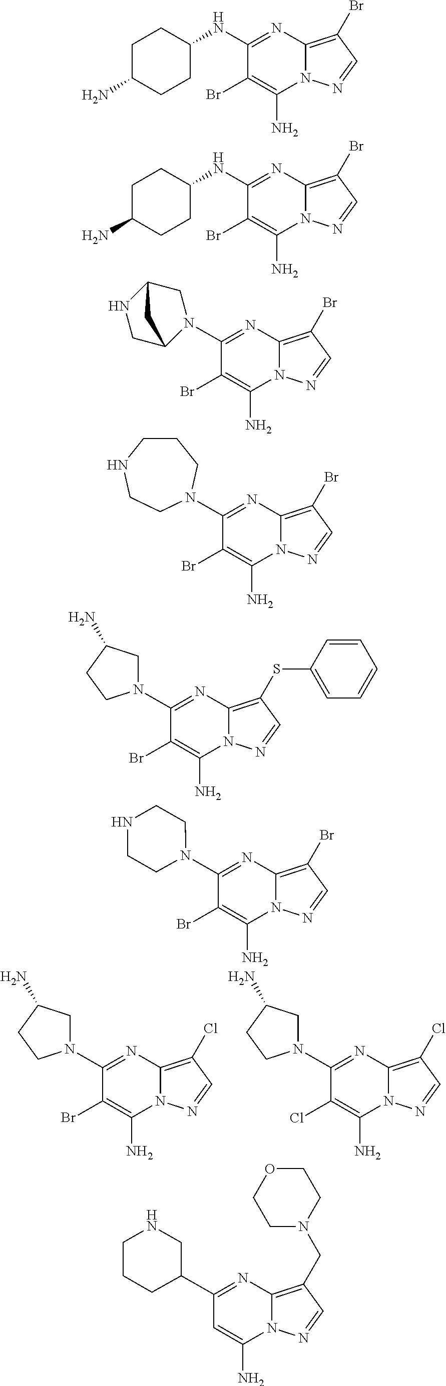

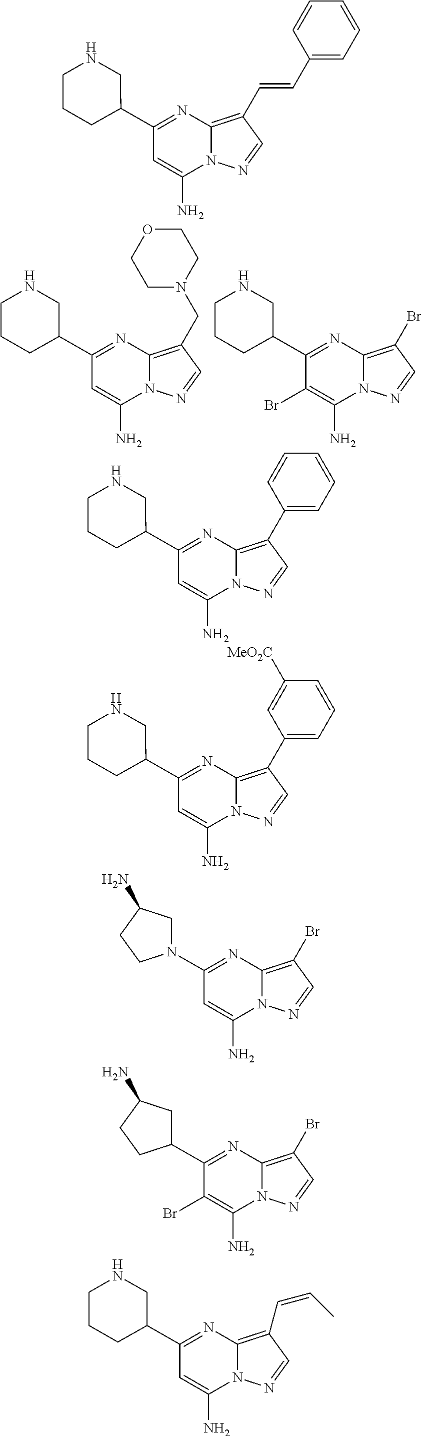

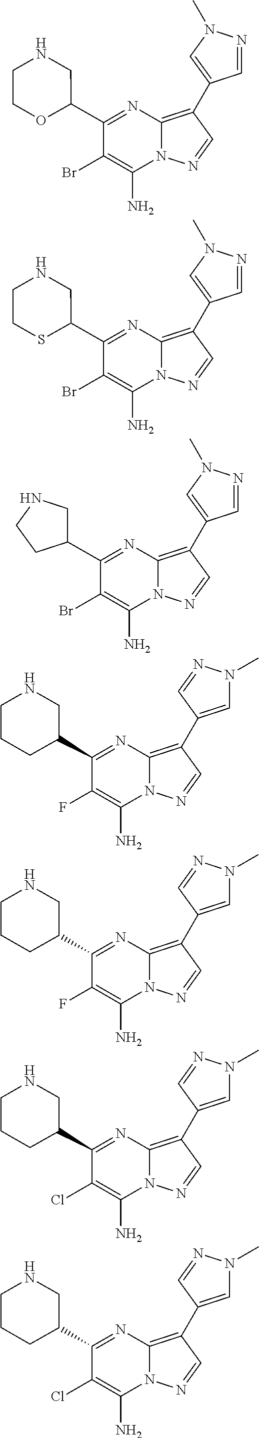

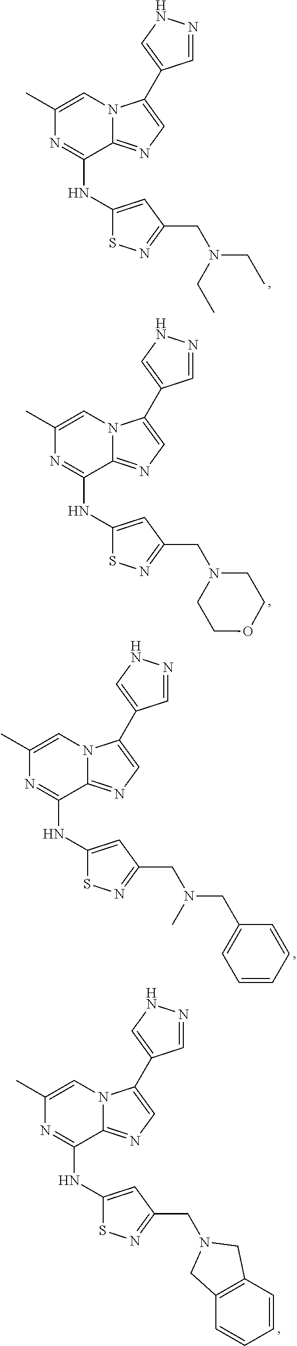

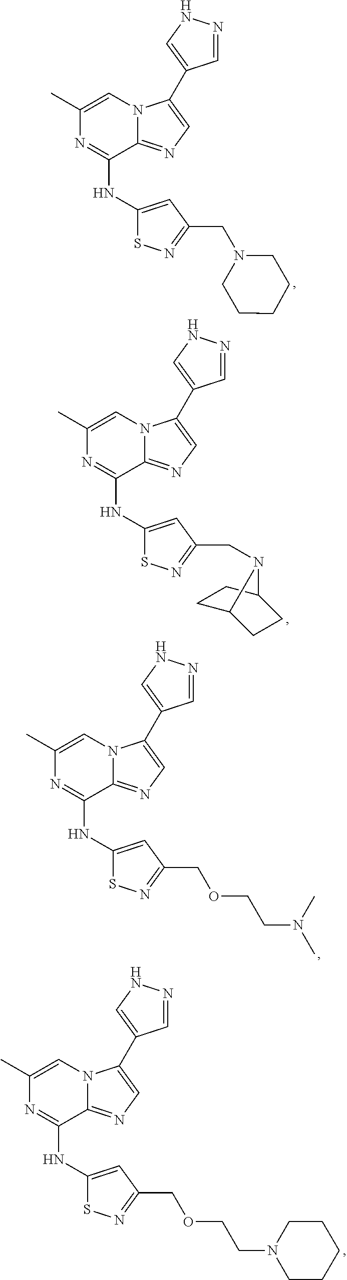

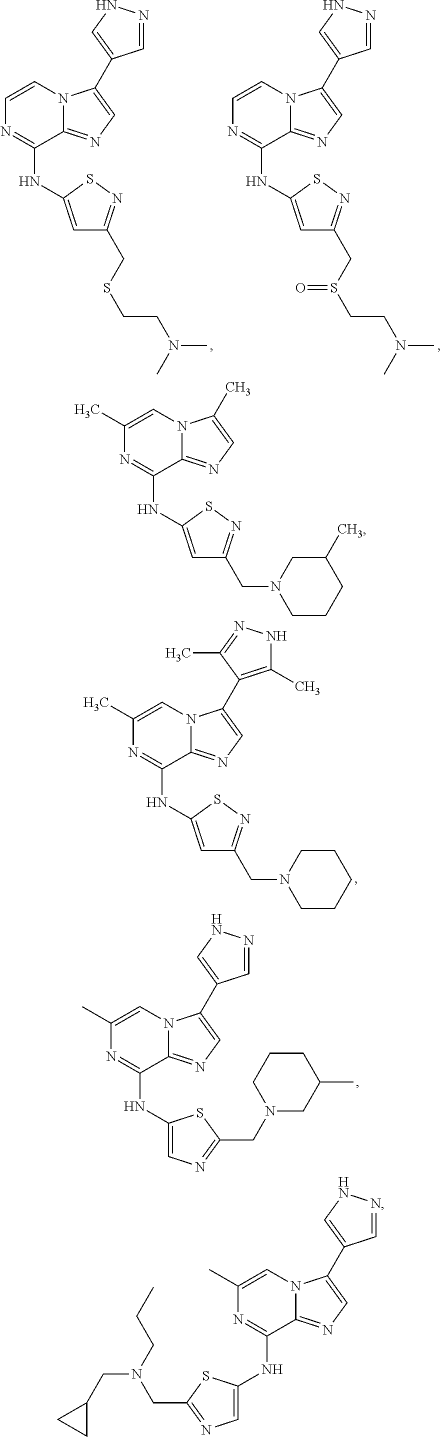

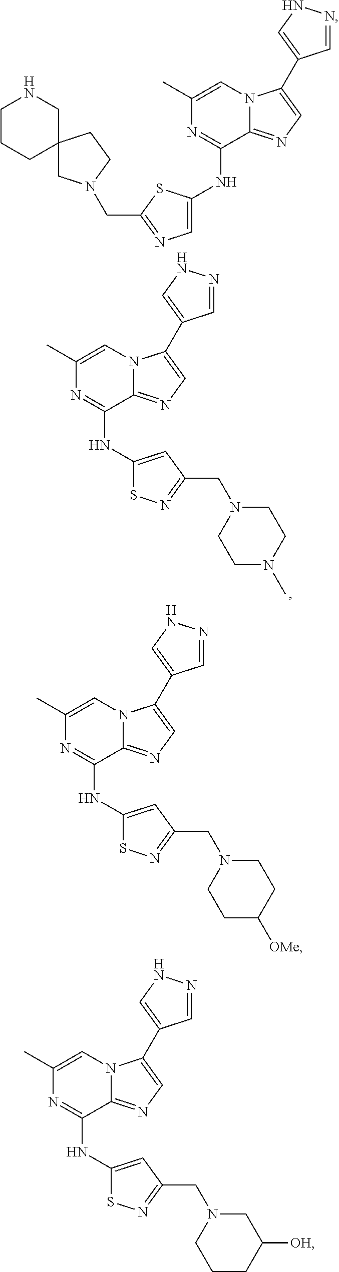

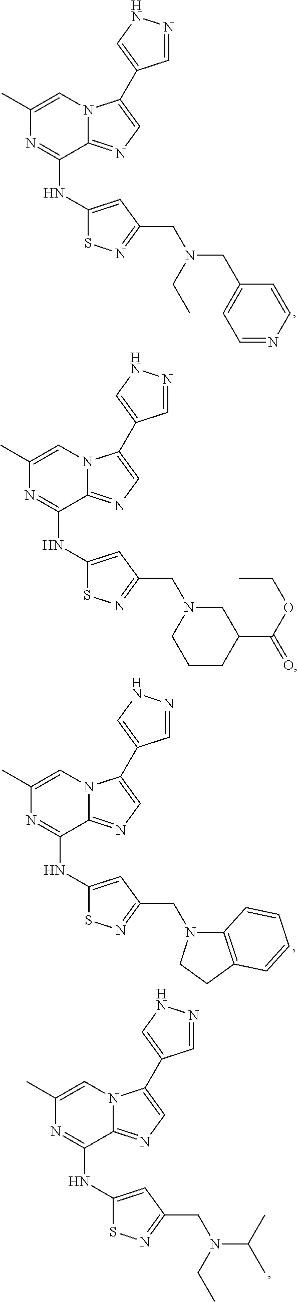

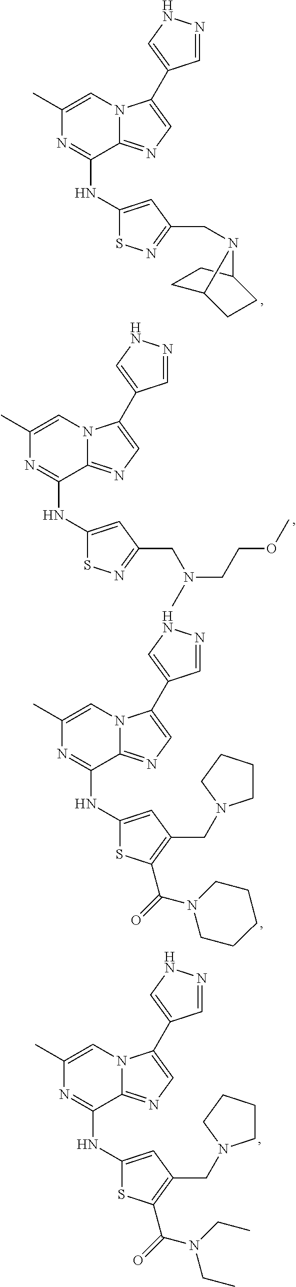

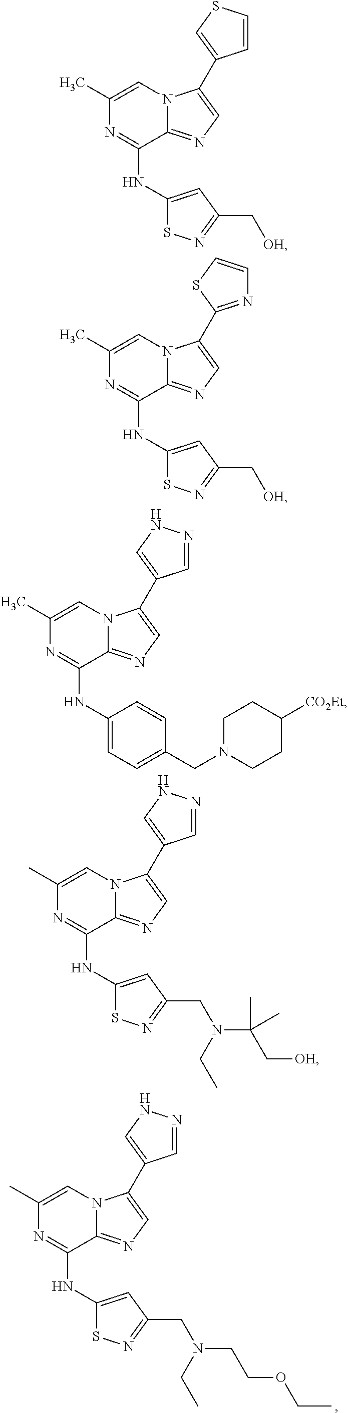

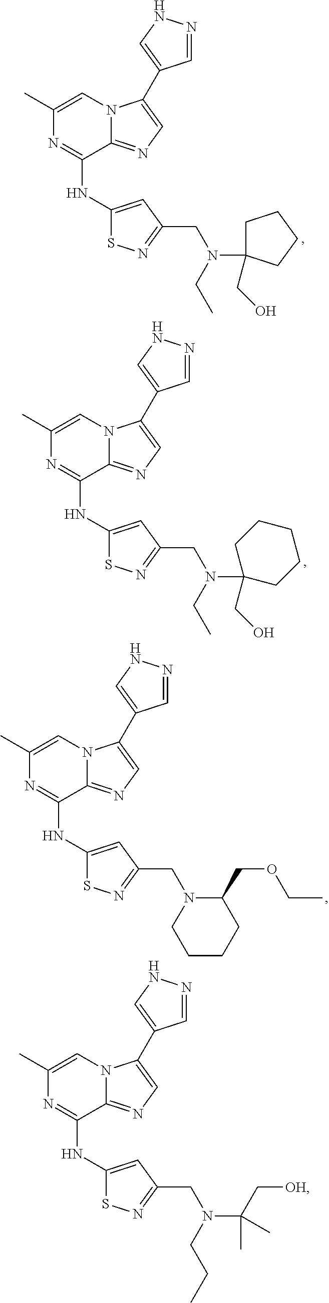

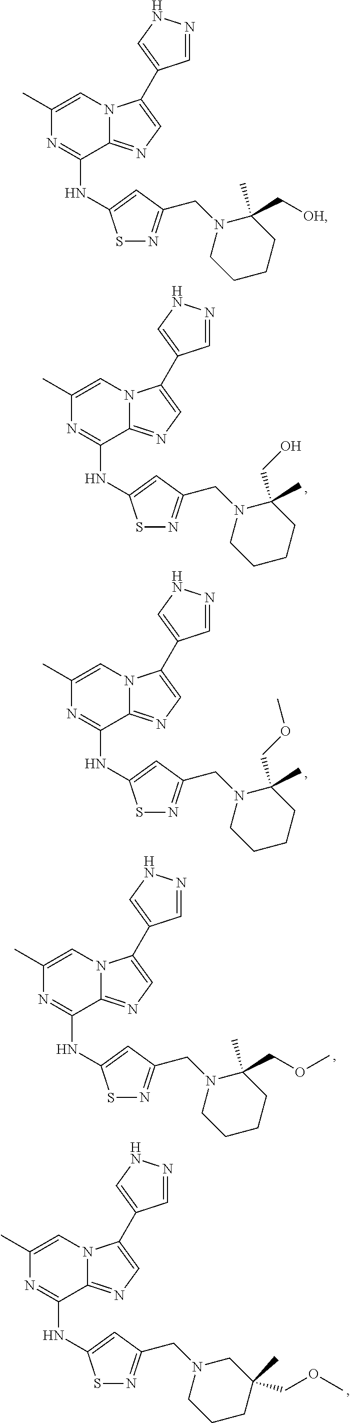

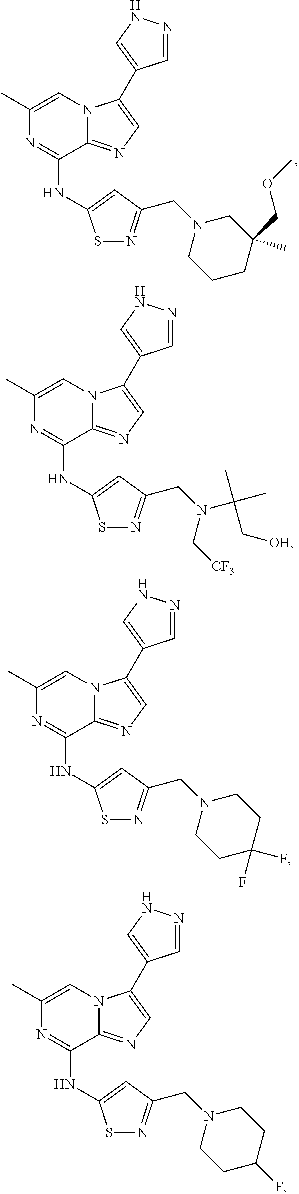

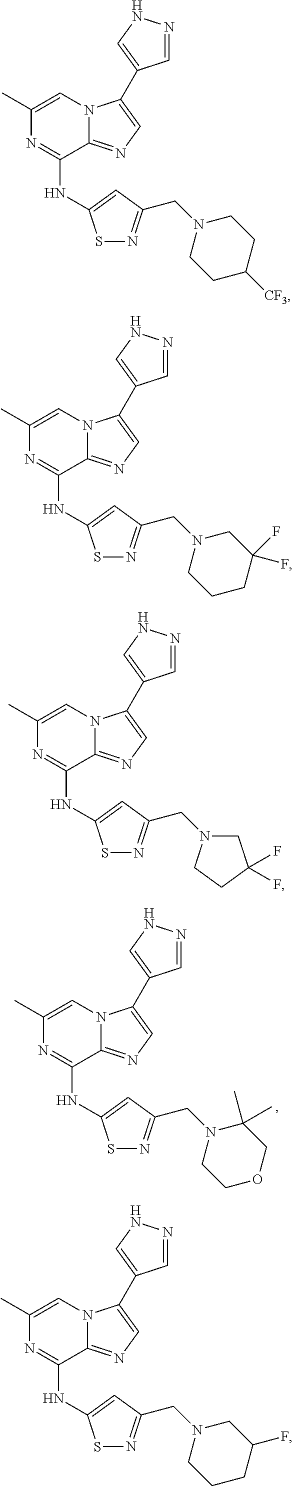

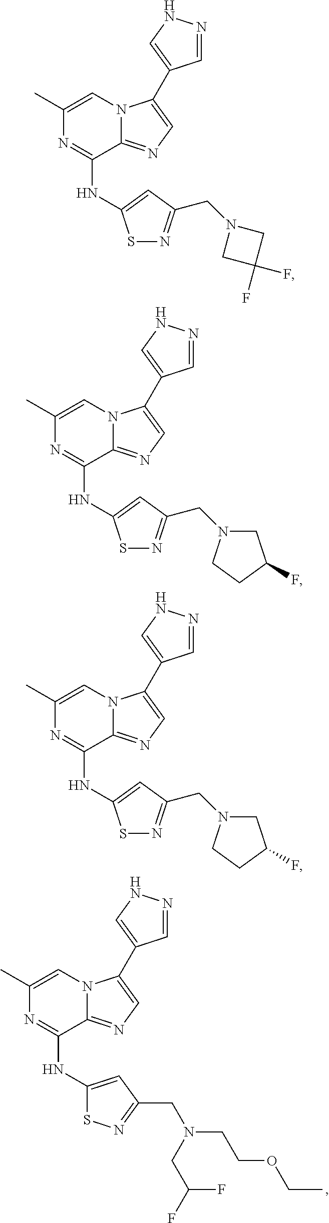

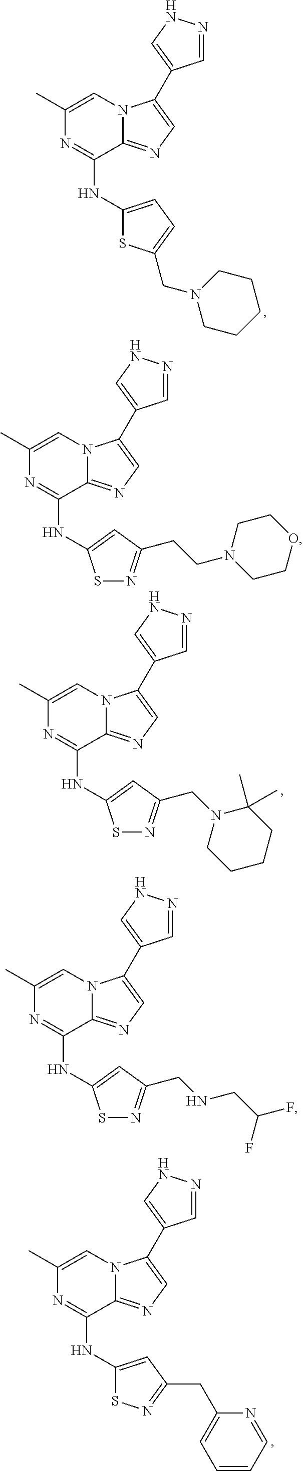

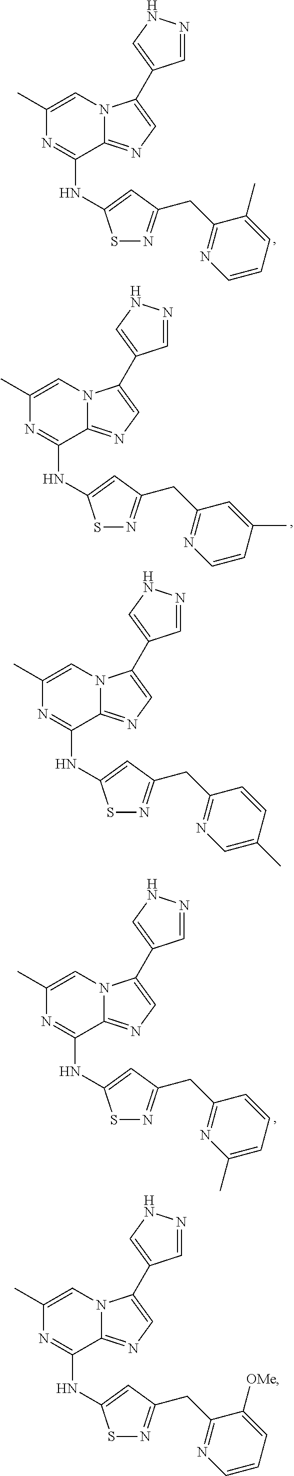

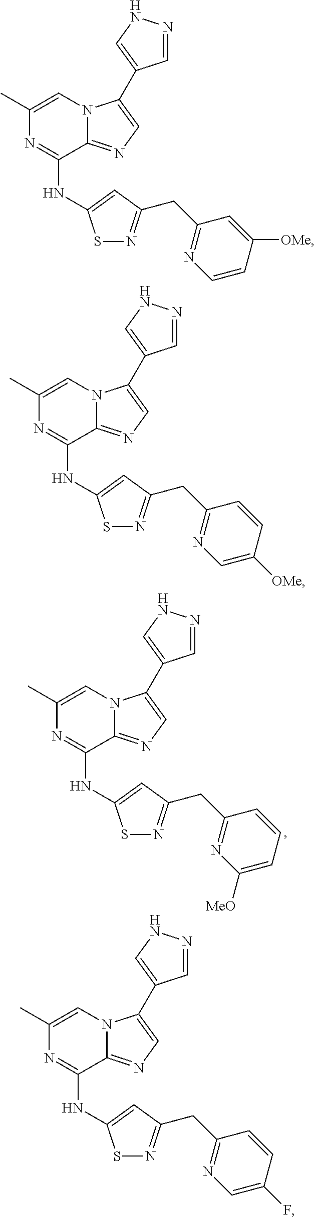

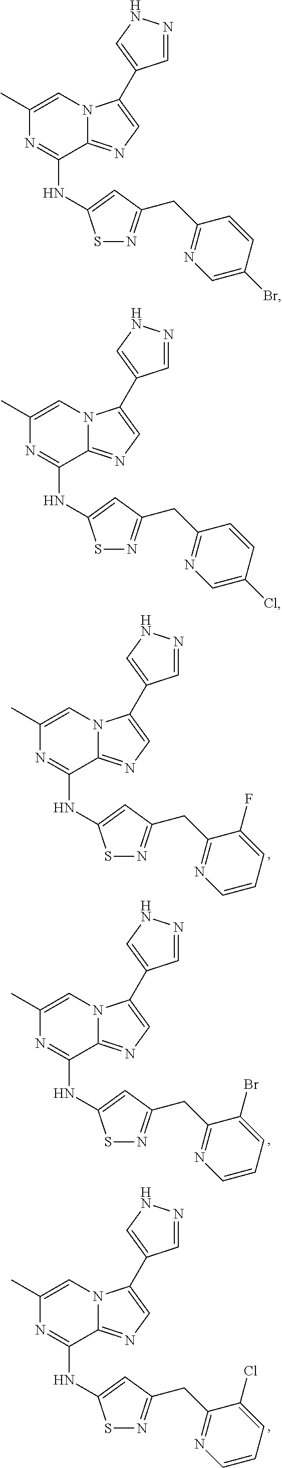

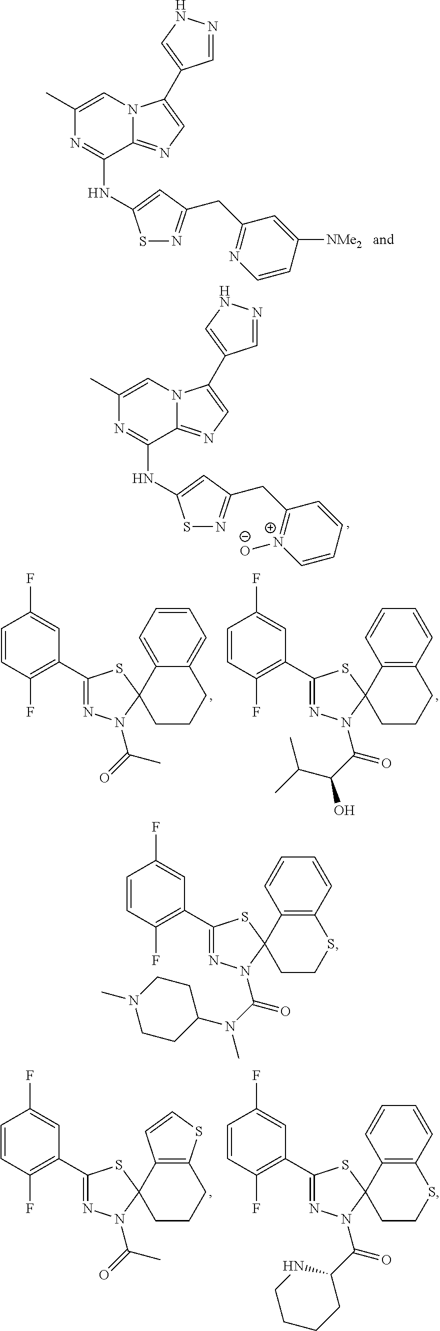

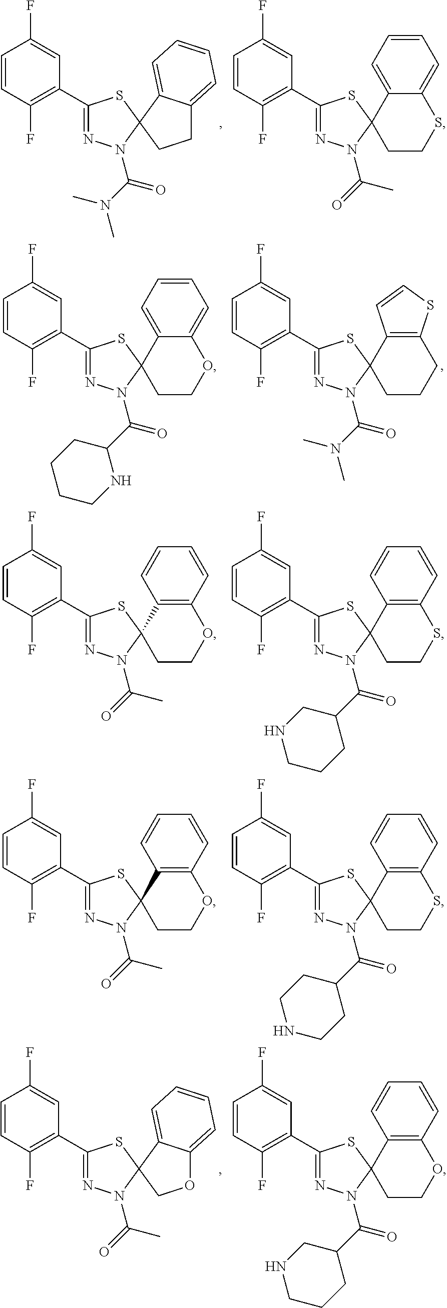

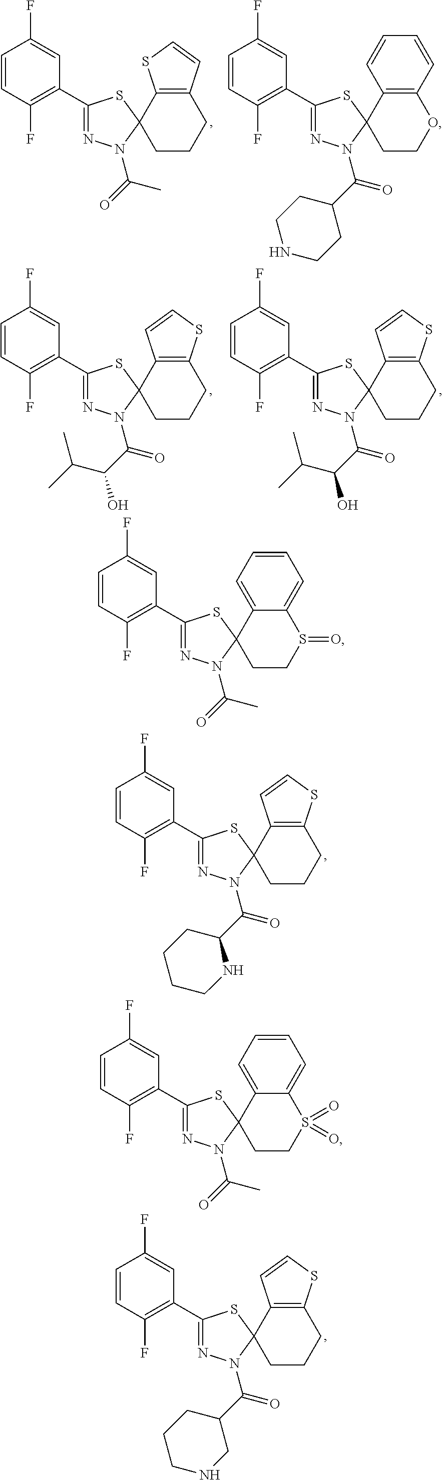

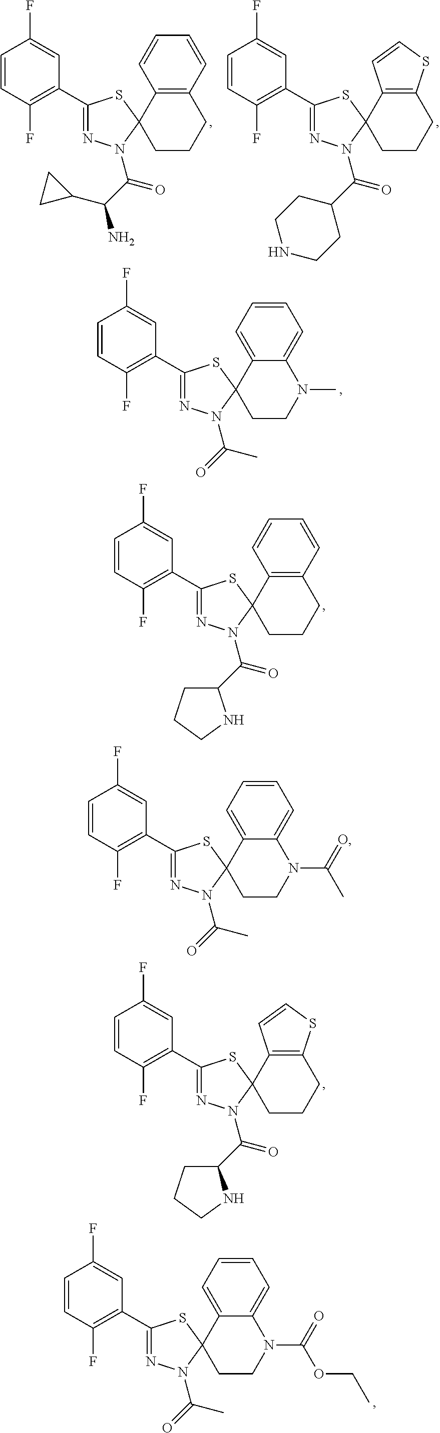

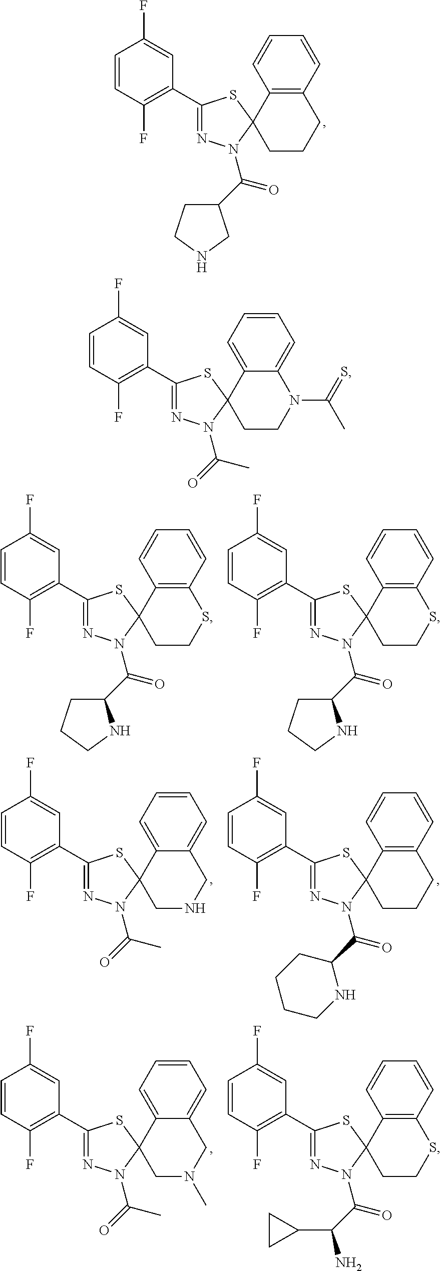

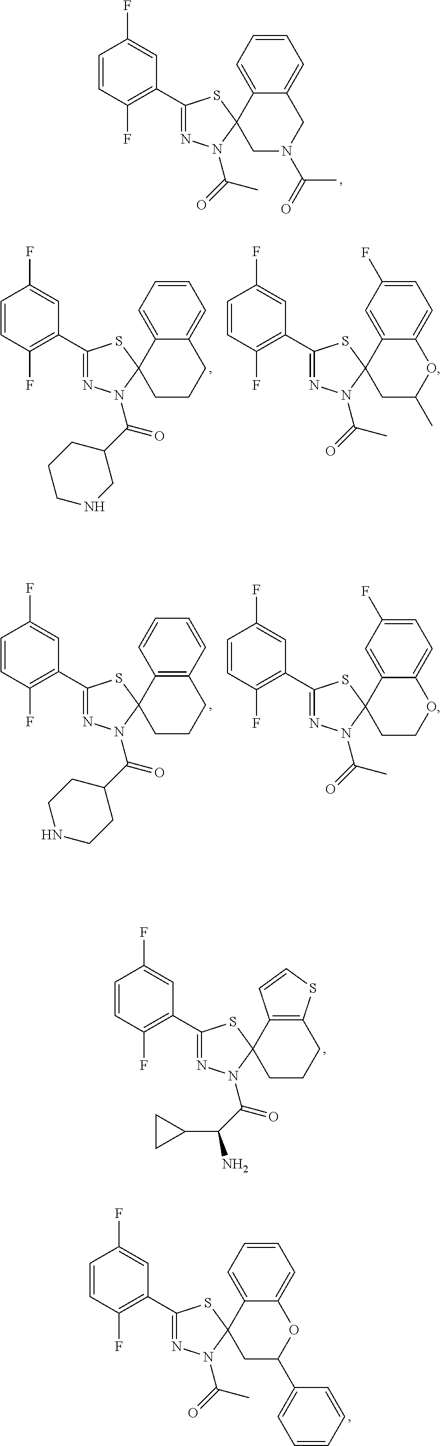

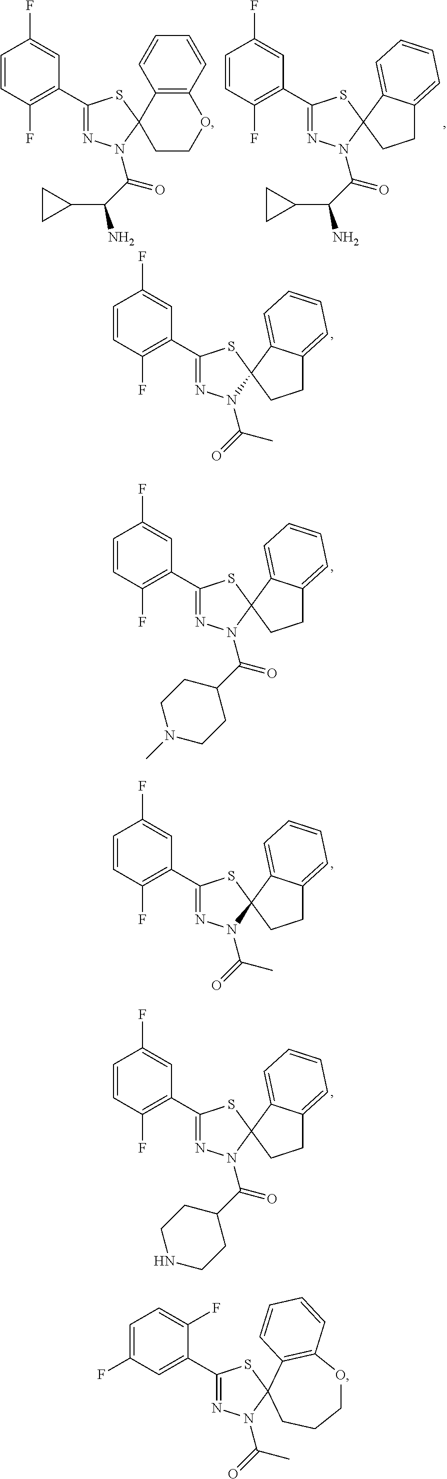

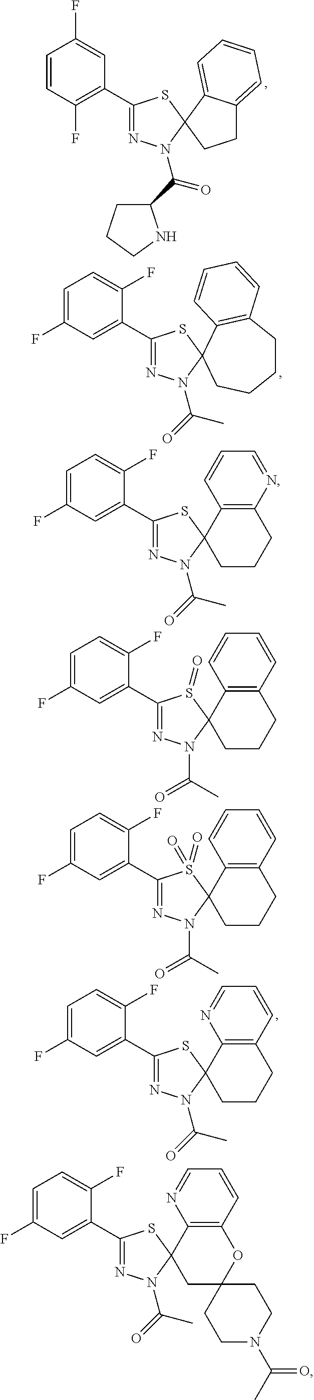

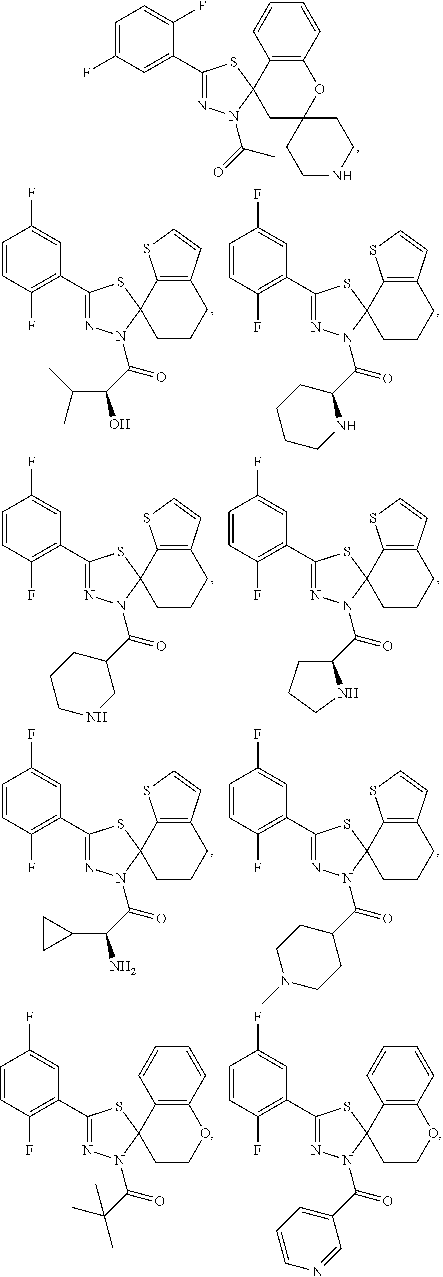

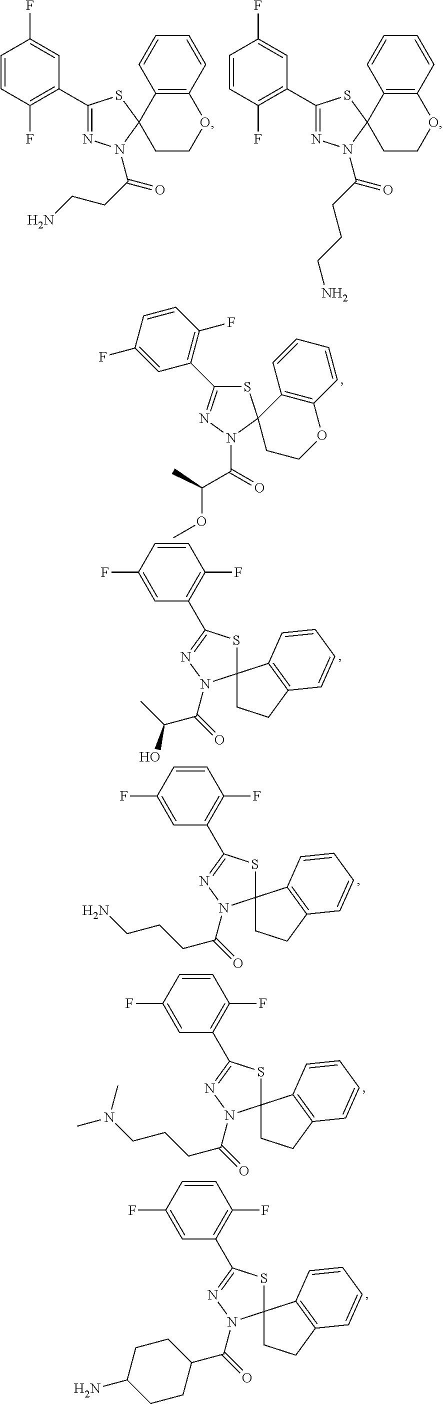

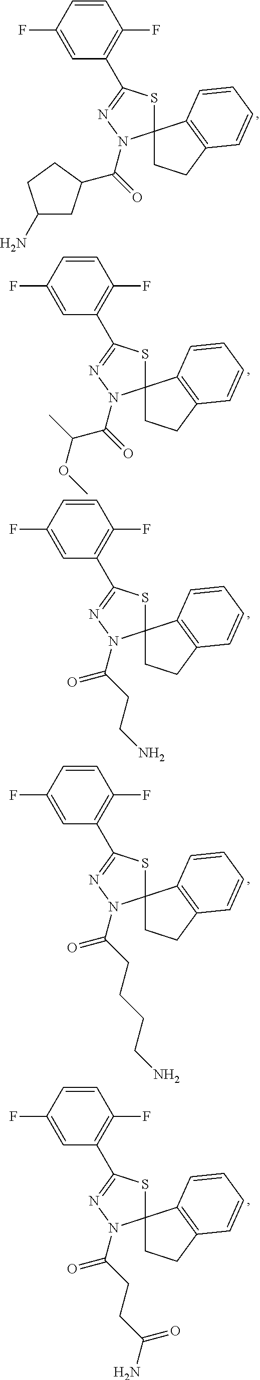

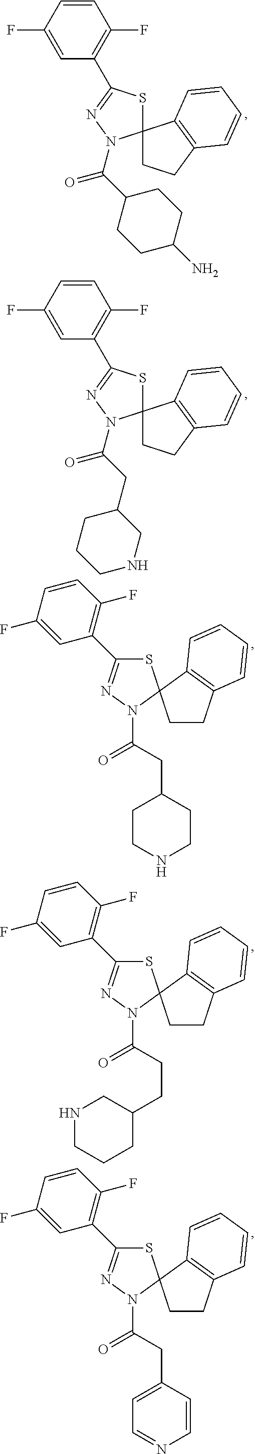

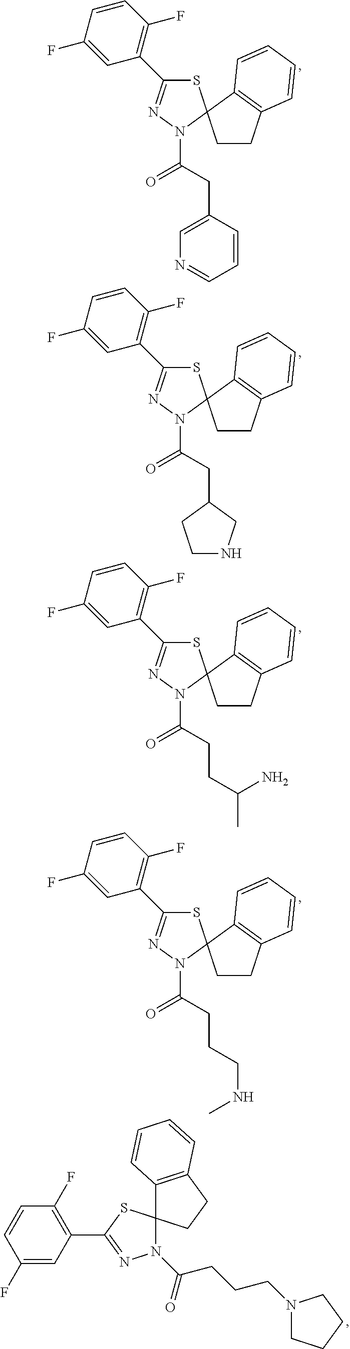

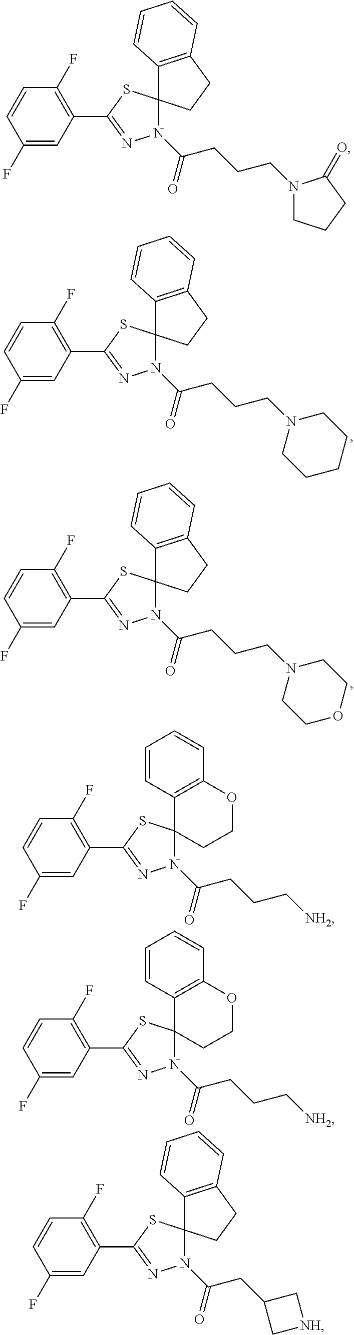

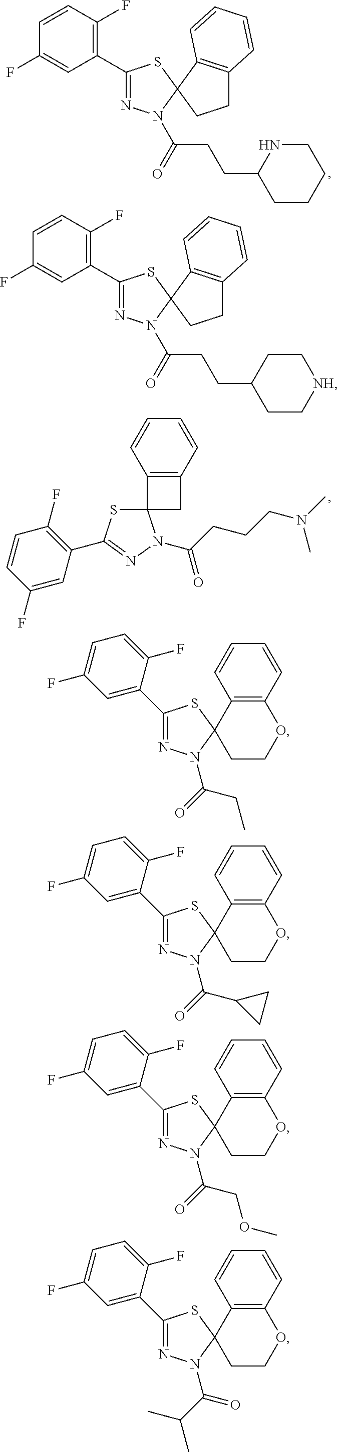

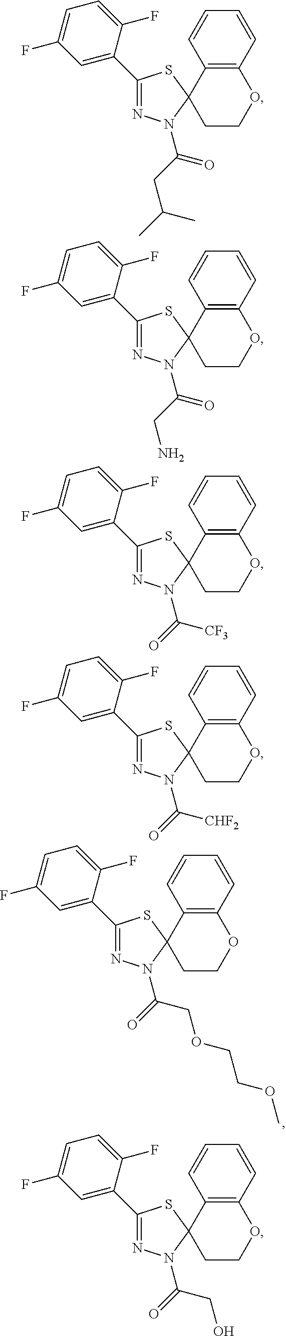

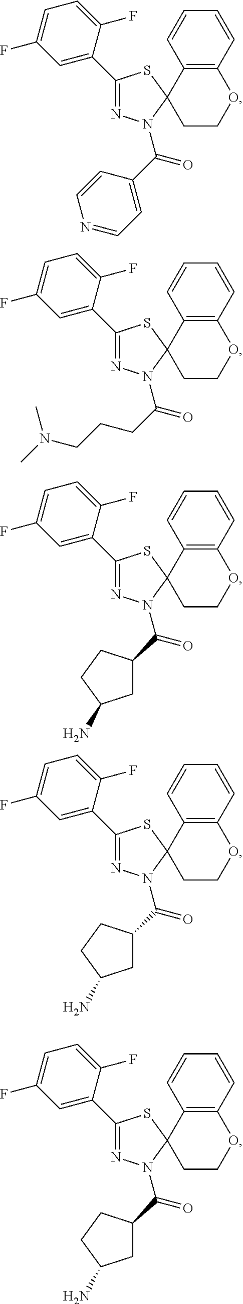

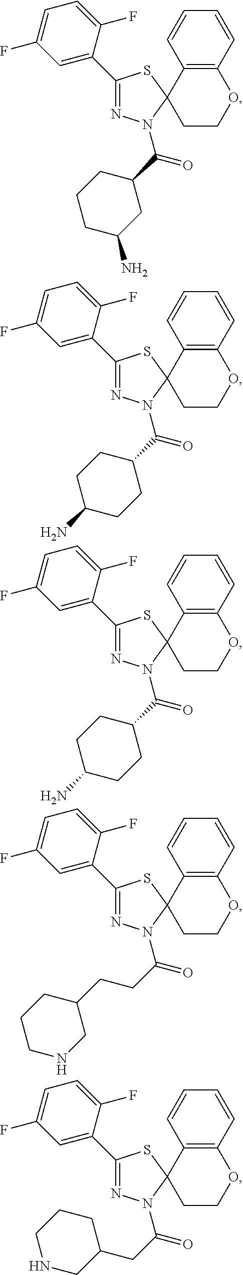

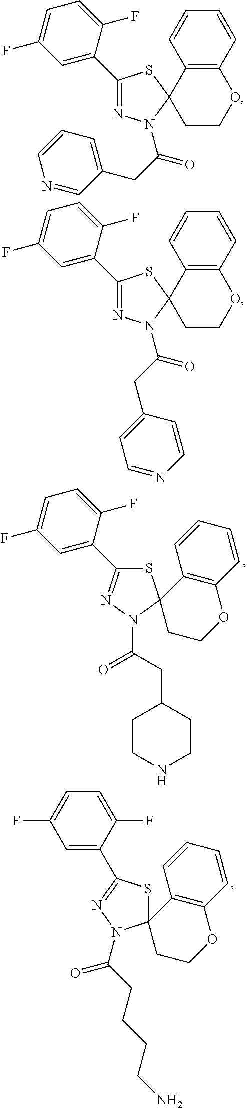

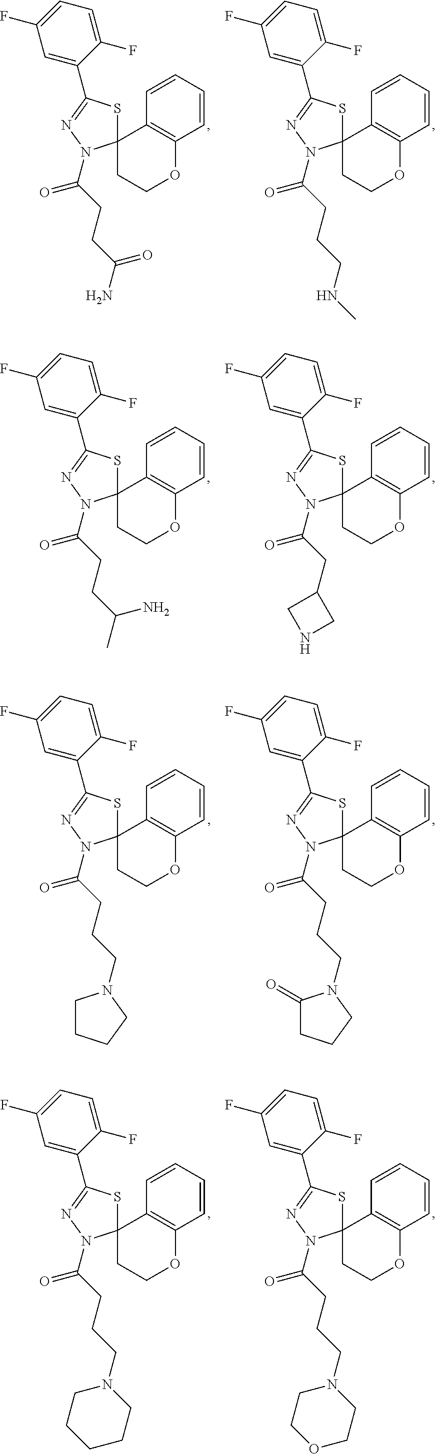

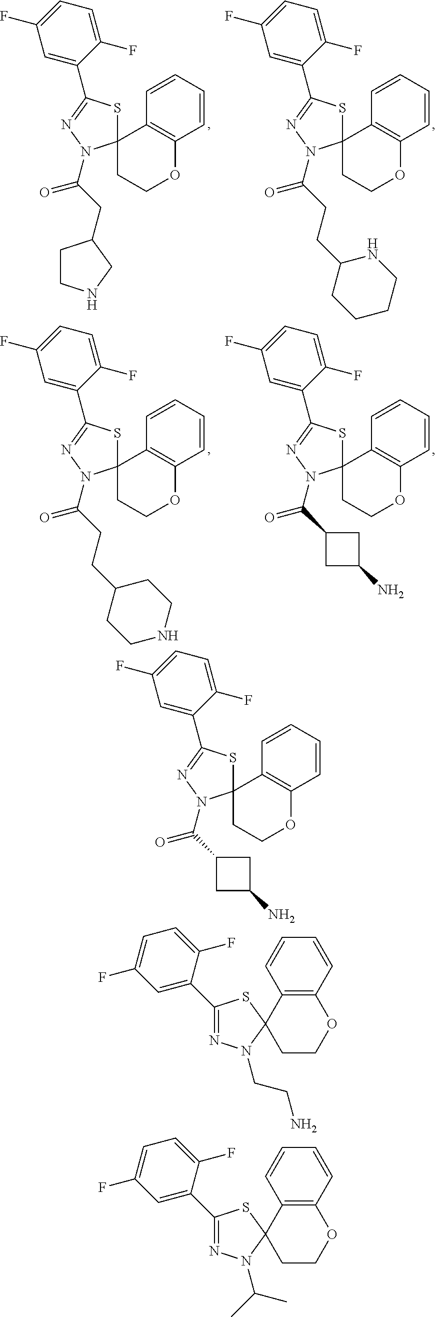

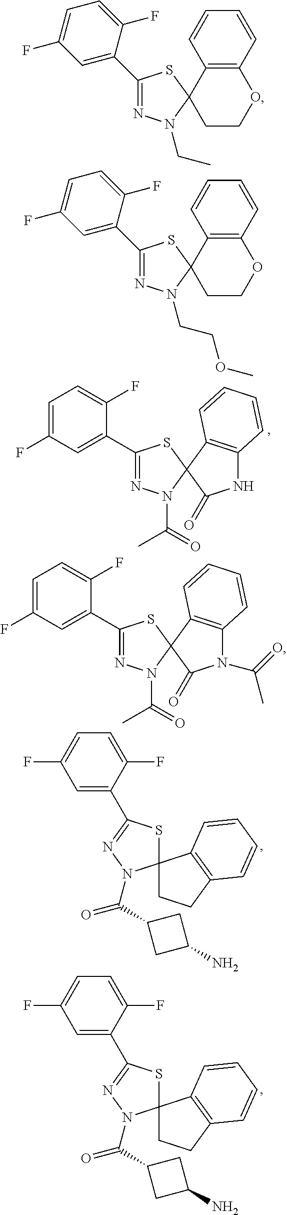

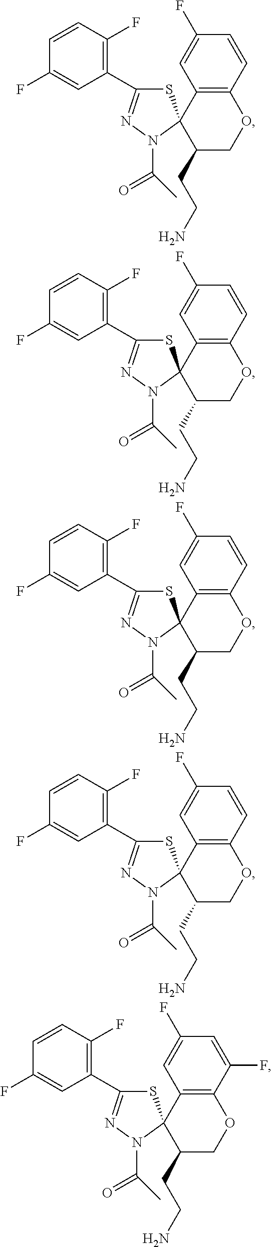

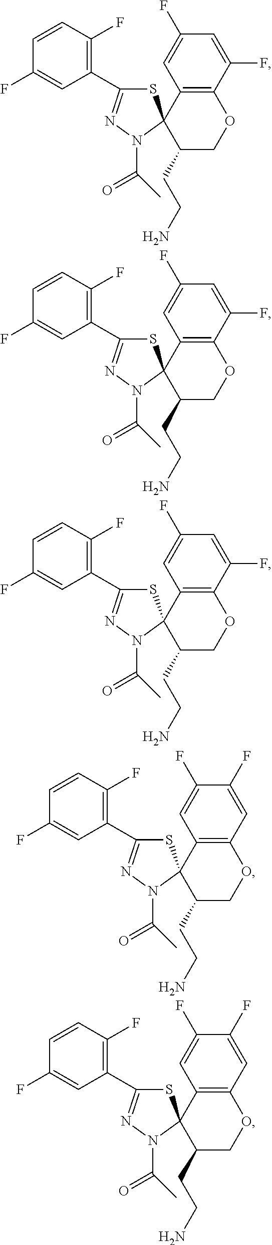

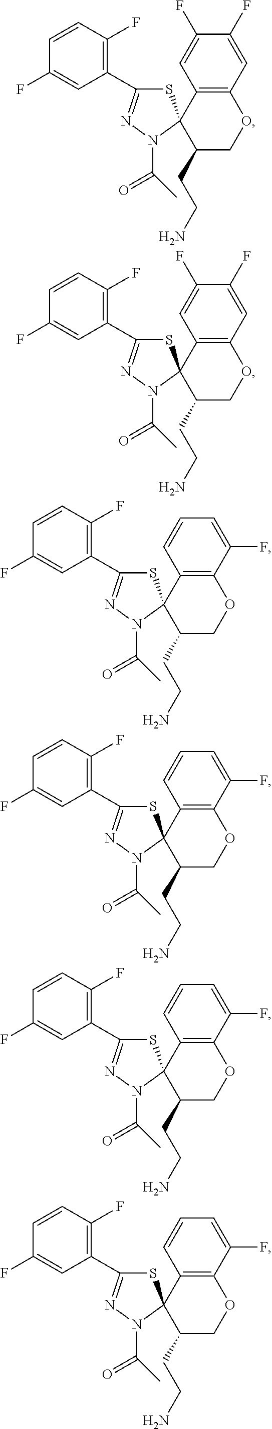

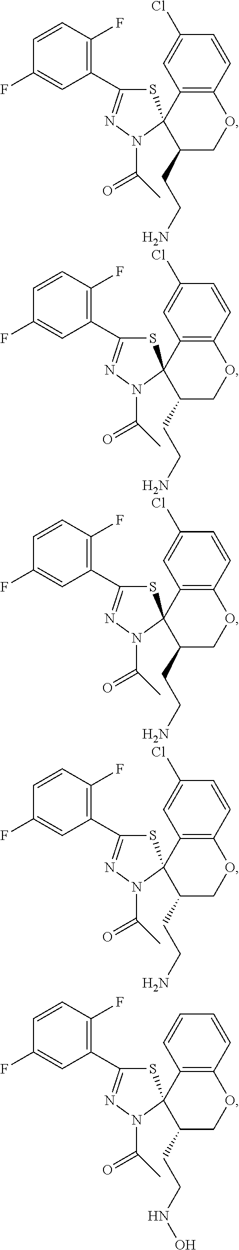

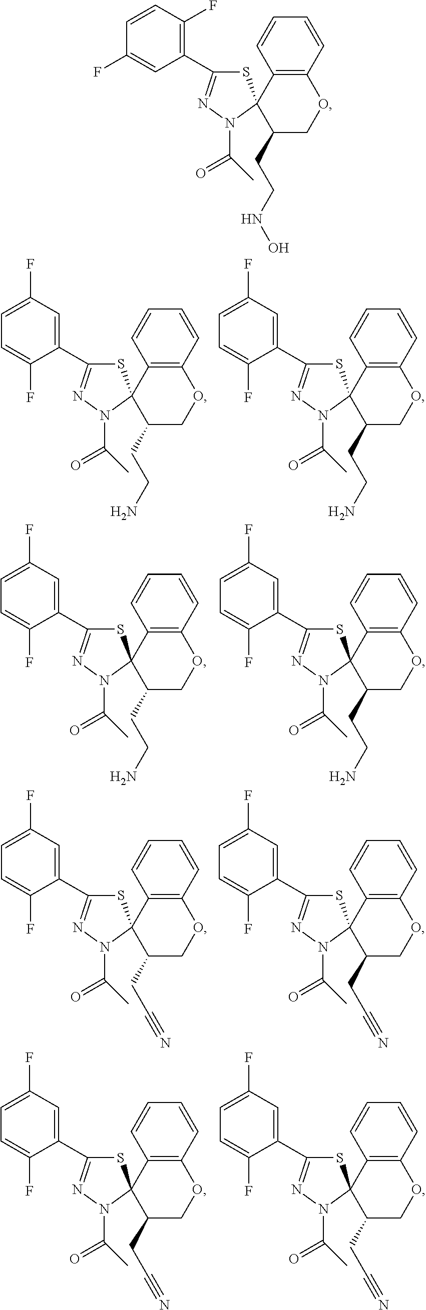

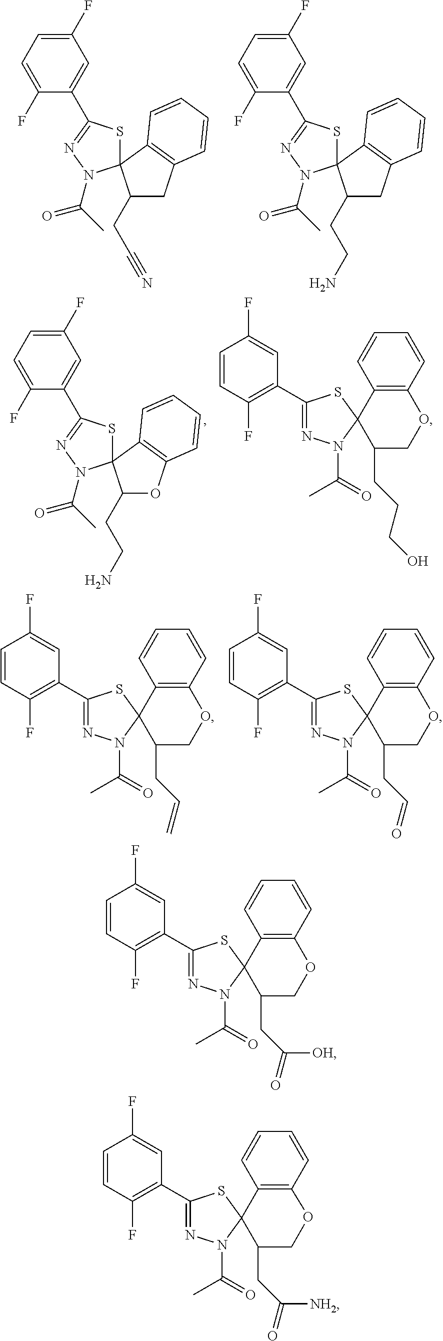

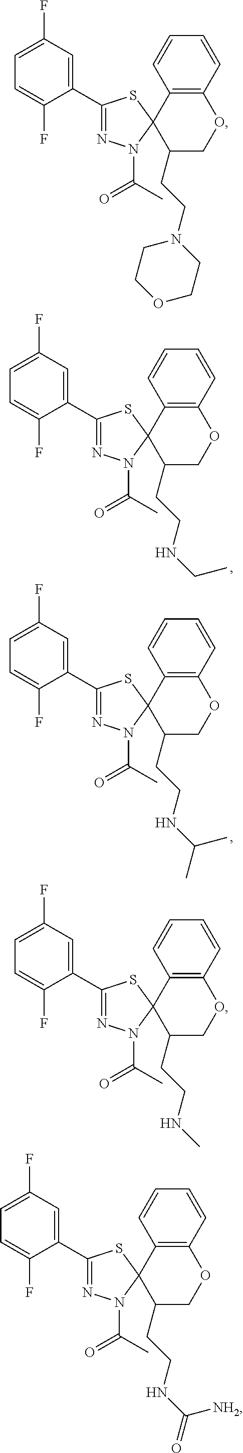

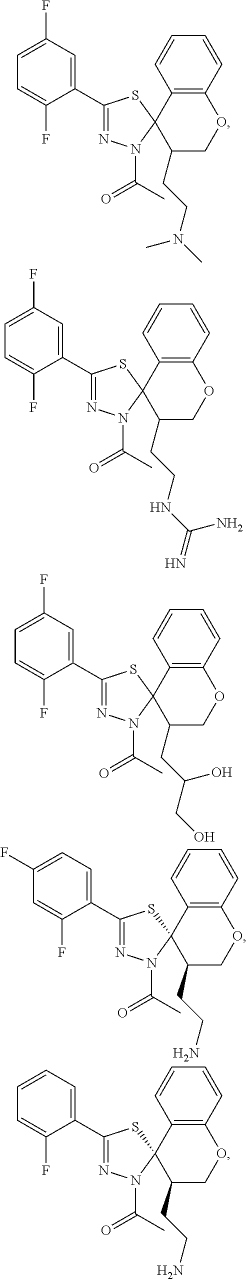

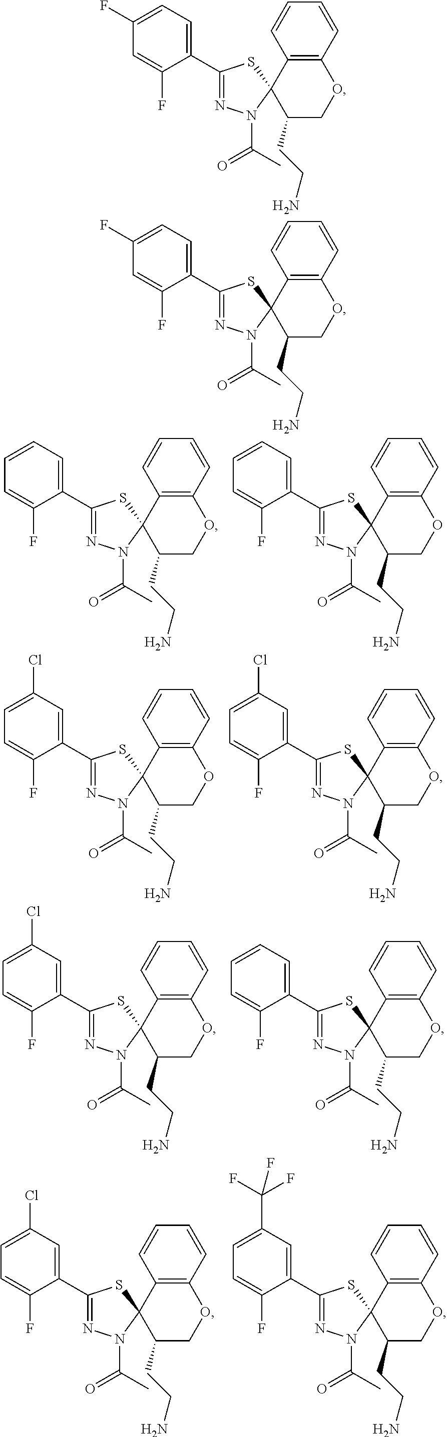

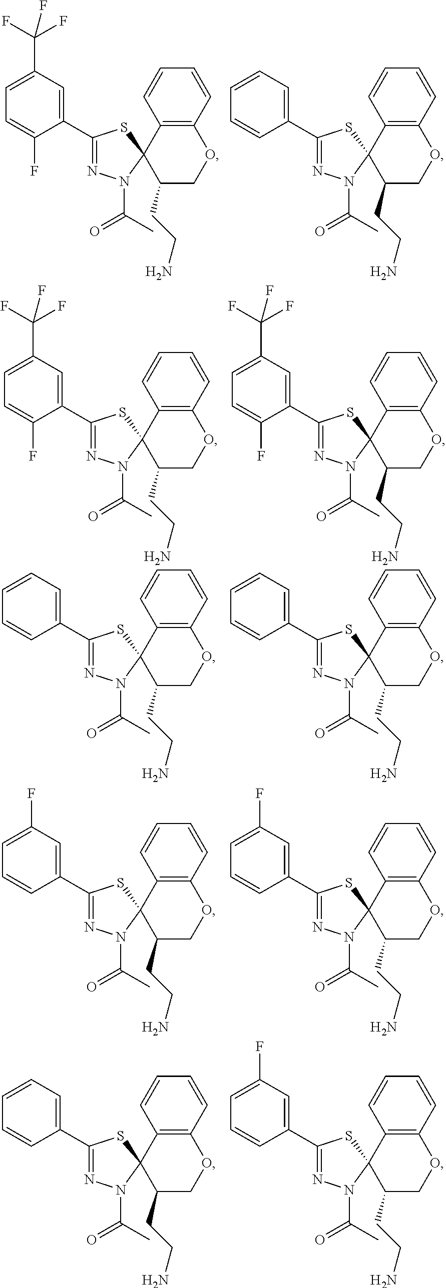

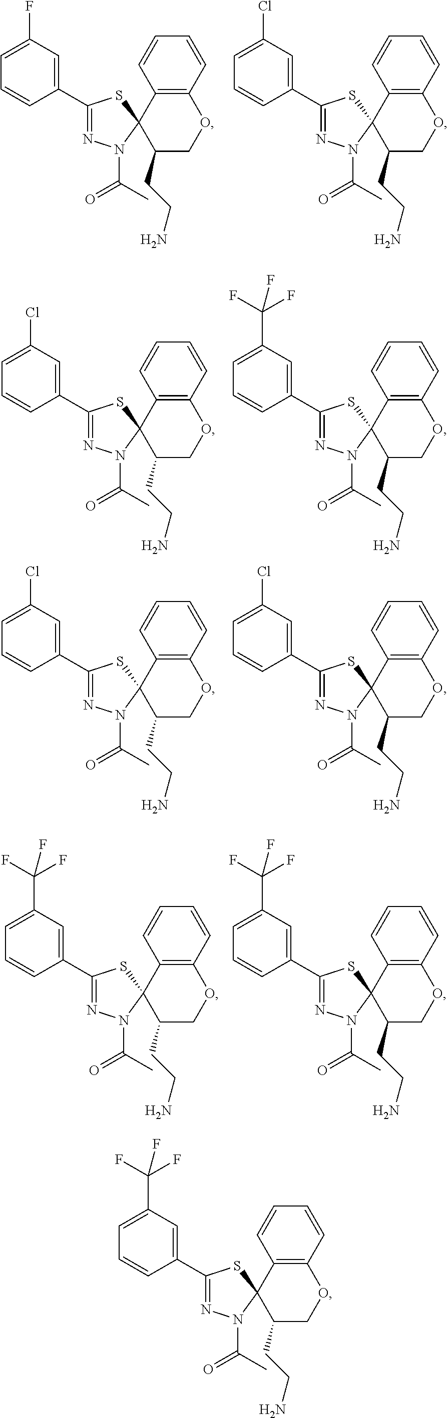

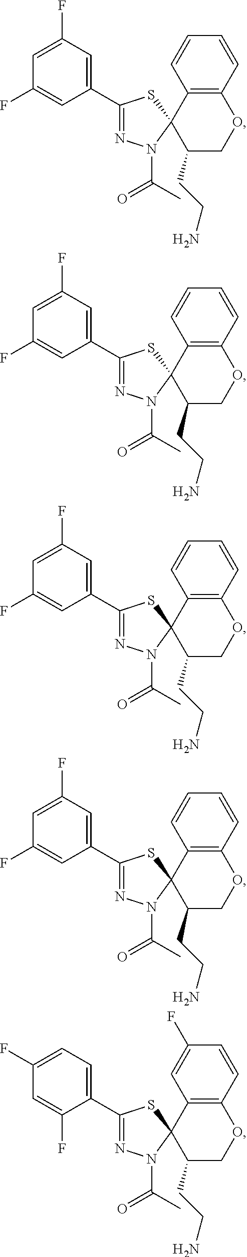

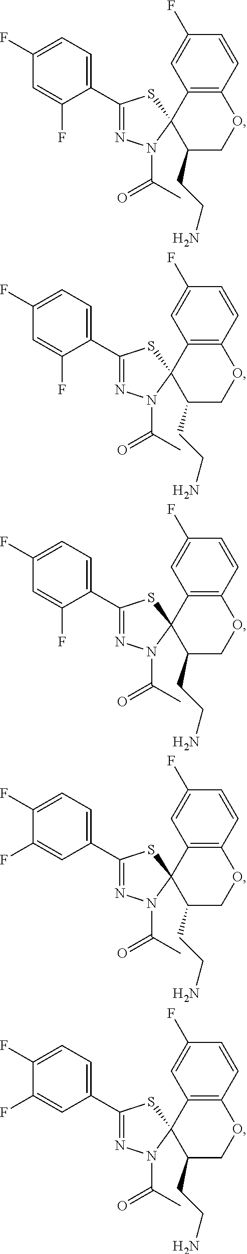

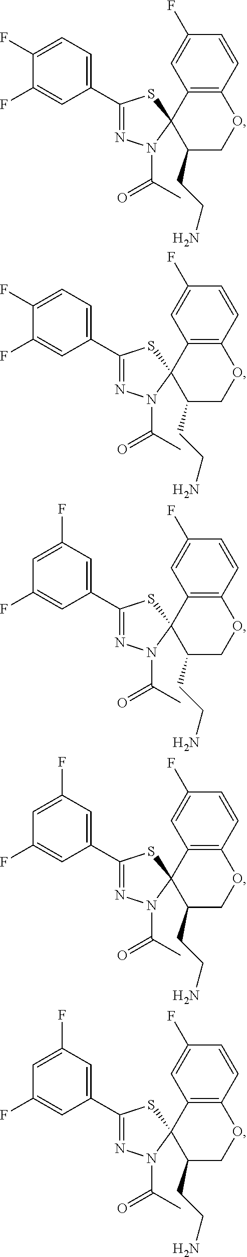

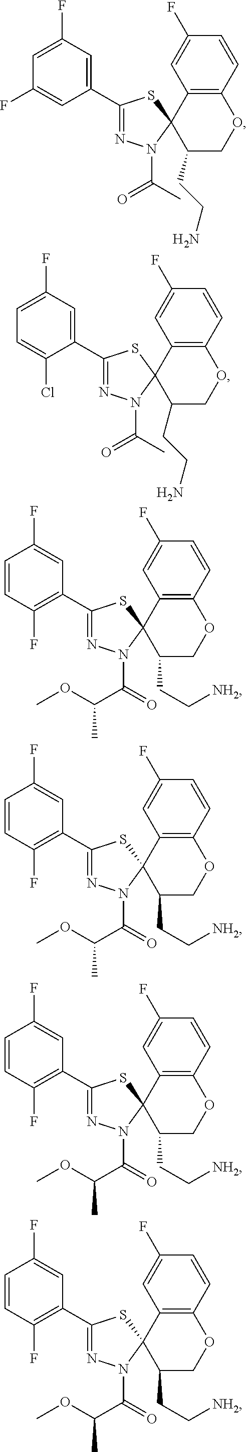

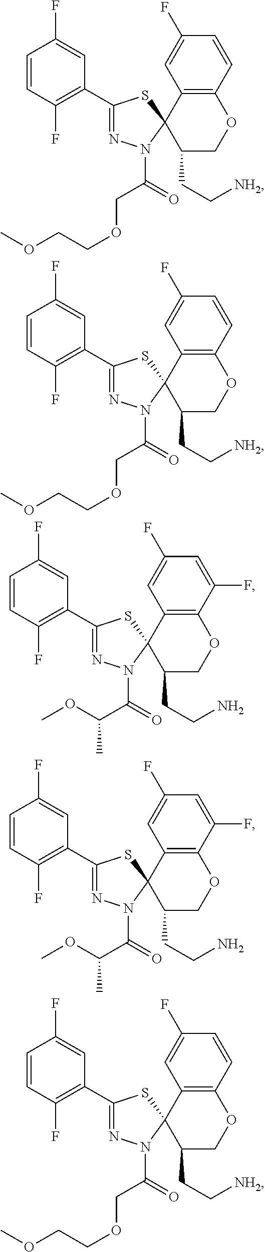

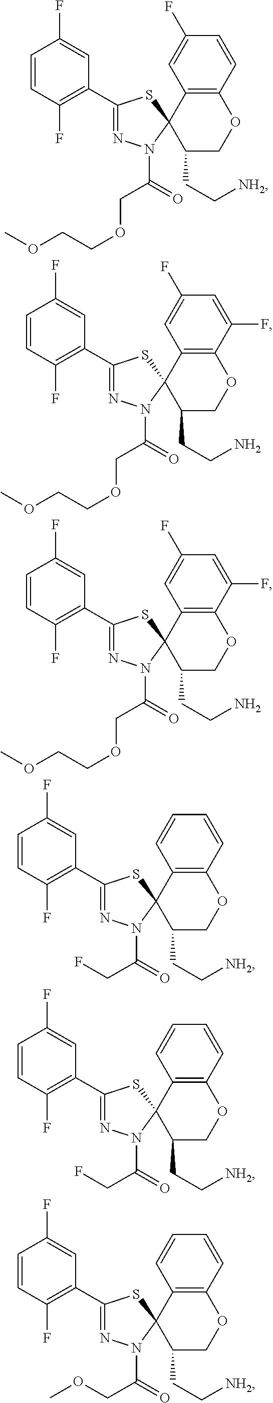

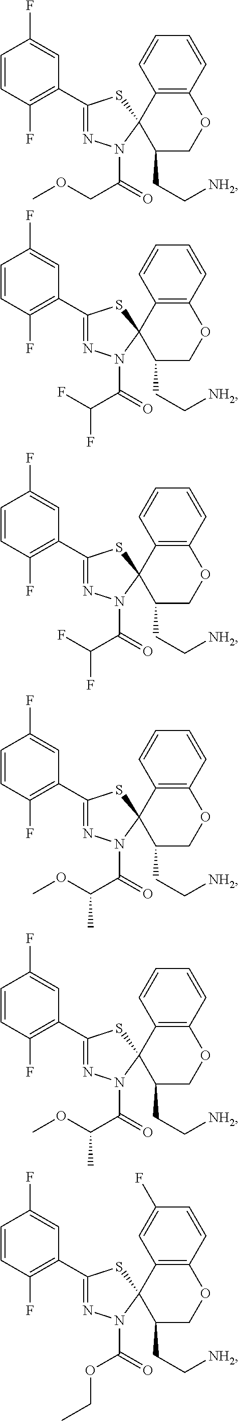

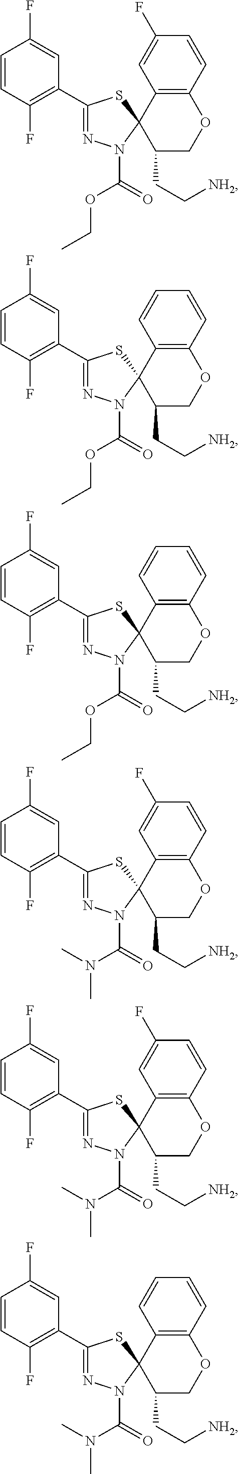

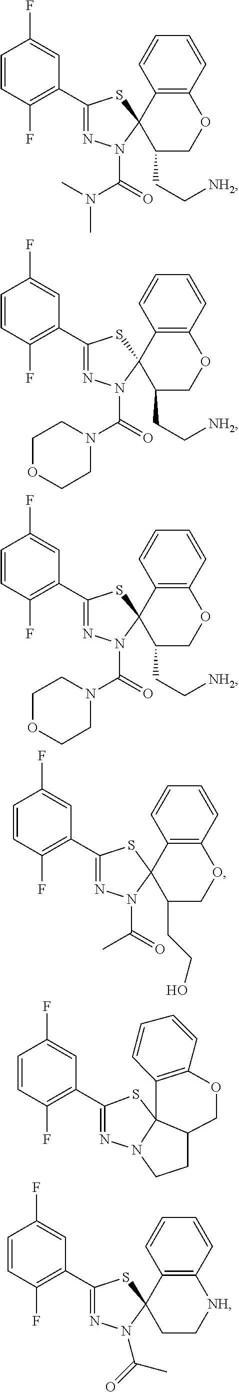

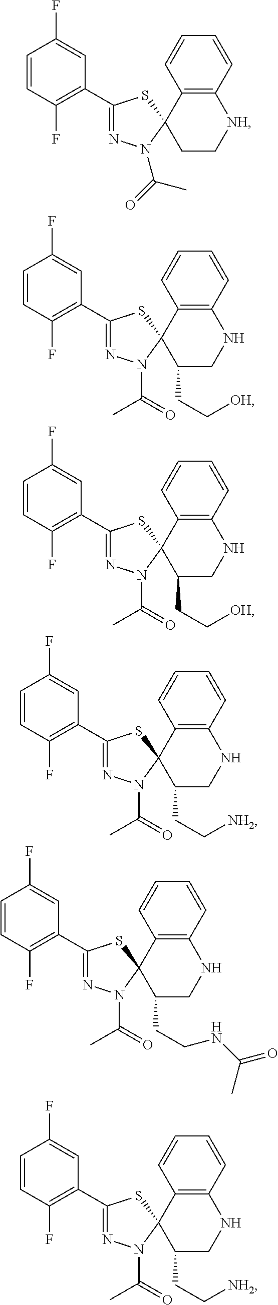

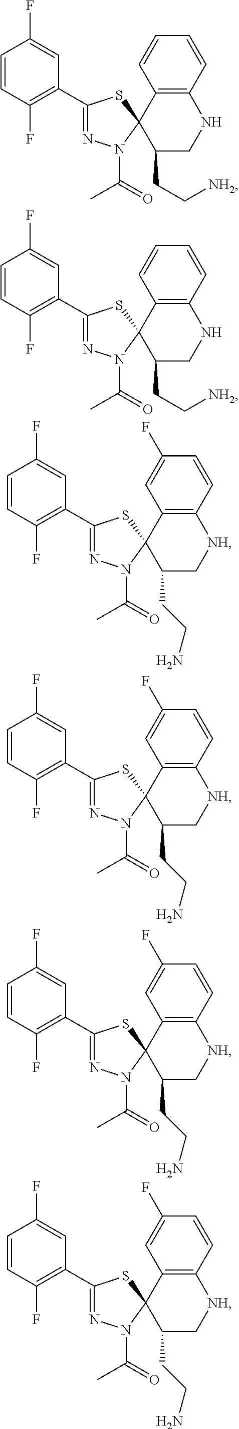

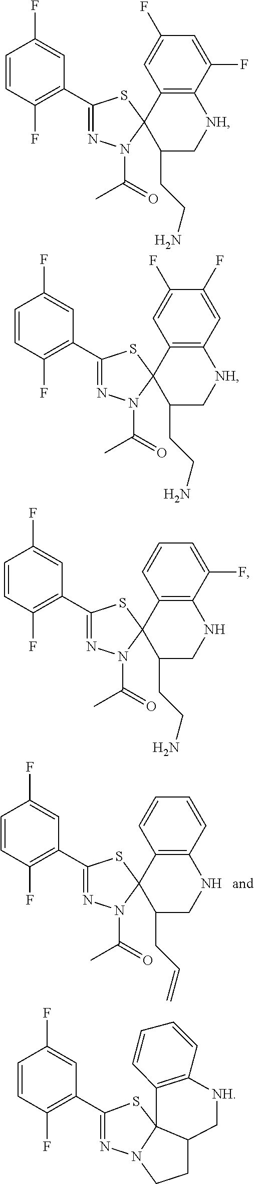

In certain embodiments, the further chemotherapeutic agent is deforolimus

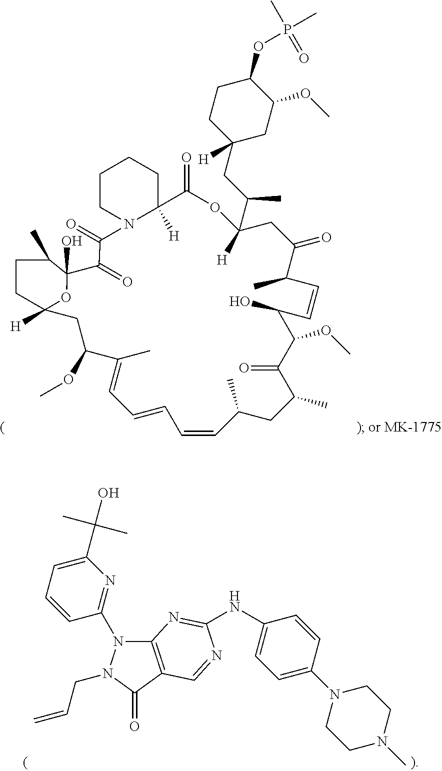

In certain embodiments, the further chemotherapeutic agent is an antibody or antigen-binding fragment thereof that specifically binds IGF1R comprising the heavy chain immunoglobulin sequence:

(SEQ ID NO: 22)
QVQLQESGPG LVKPSETLSL TCTVSGYSIS GGYLWNWIRQ
PPGKGLEWIG YISYDGTNNY KPSLKDRVTI SVDTSKNQFS
LKLSSVTAAD TAVYYCARYG RVFFDYWGQG TLVTVSS;
and/or

the light chain immunoglobulin sequence:

(SEQ ID NO: 23)
DIVMTQSPLS LPVTPGEPAS ISCRSSQSIV HSNGNTYLQW
YLQKPGQSPQ LLIYKVSNRL YGVPDRFSGS GSGTDFTLKI
SRVEAEDVGV YYCFQGSHVP WTFGQGTKVE IK;
or in

combination with any antibody or antigen-binding fragment that comprises the light chain and/or the heavy chain CDRs set forth above.

In certain embodiments, the further chemotherapeutic agent is an ERK inhibitor selected from the group consisting of:

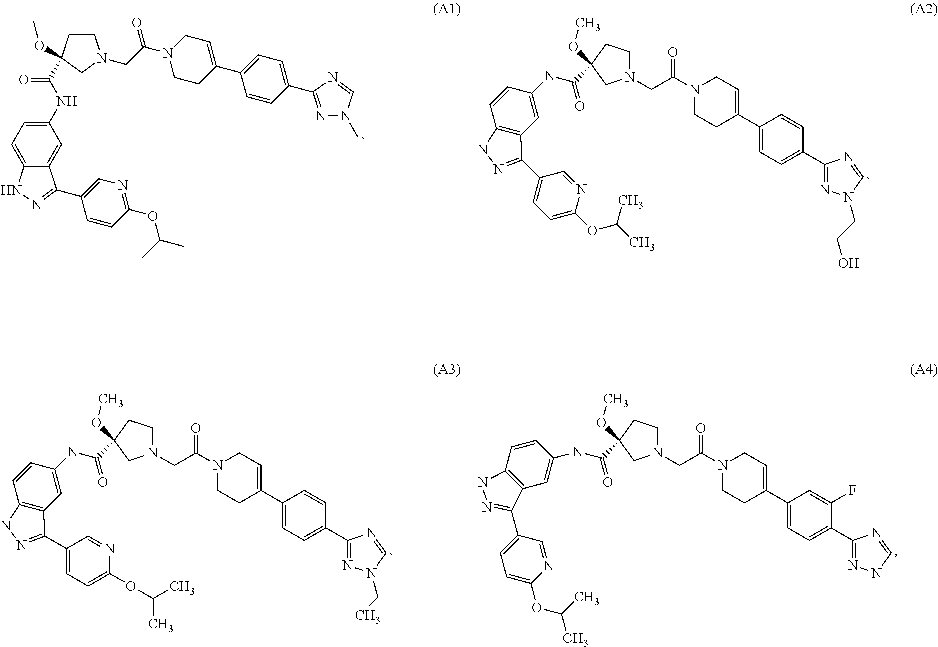

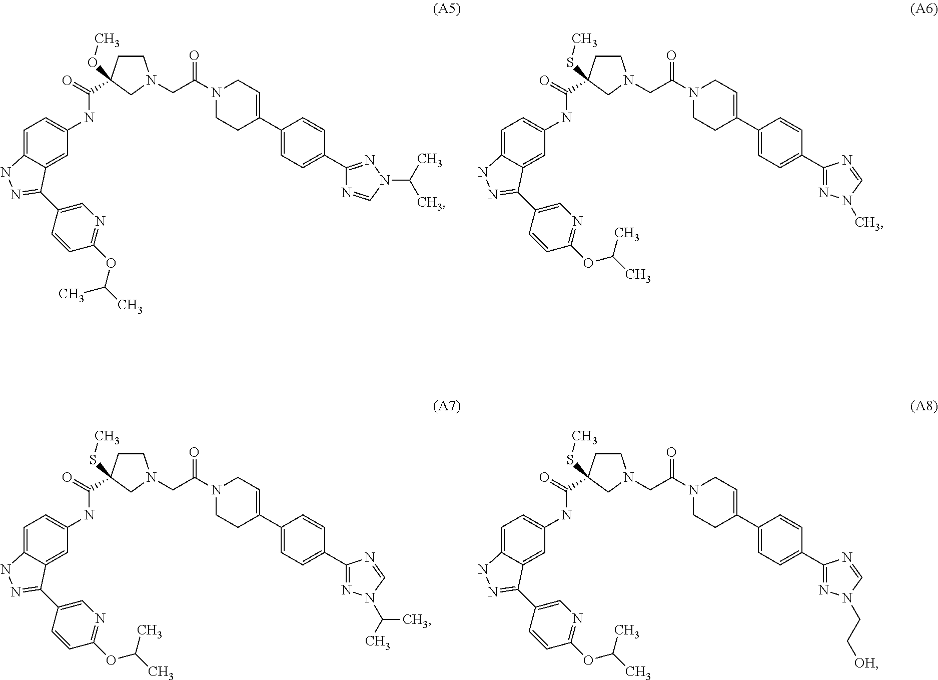

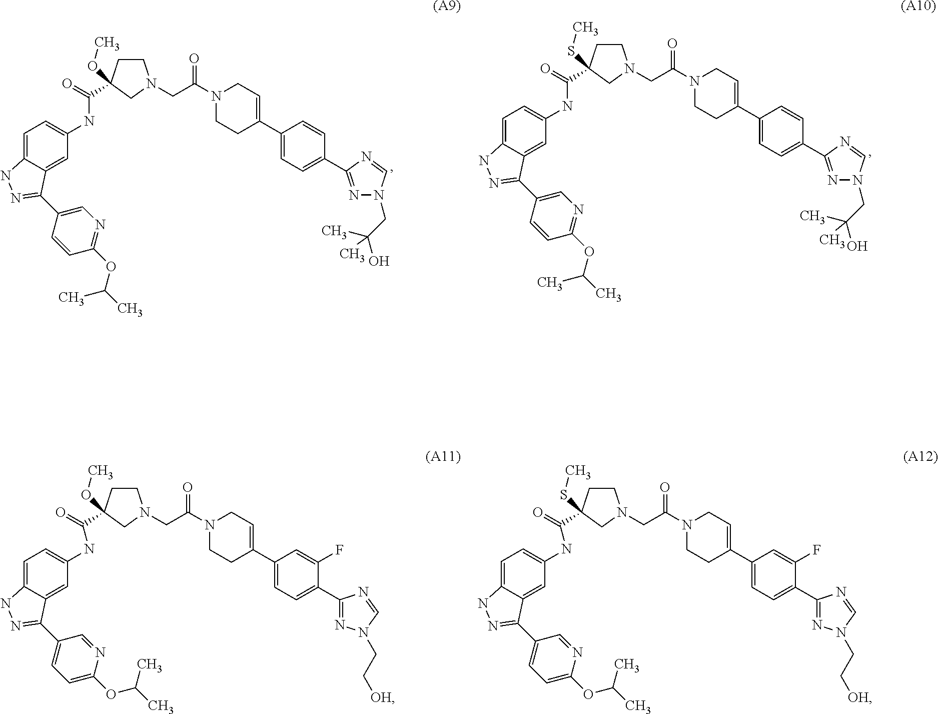

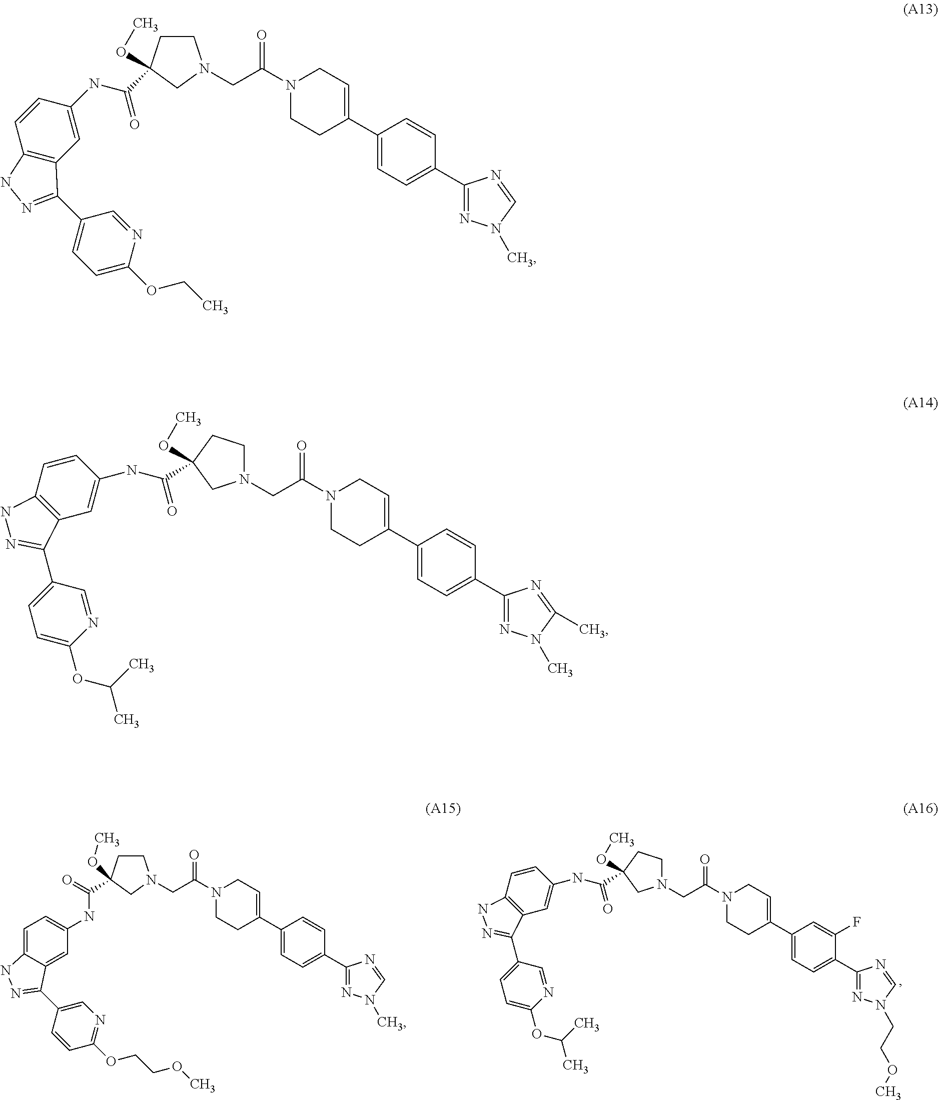

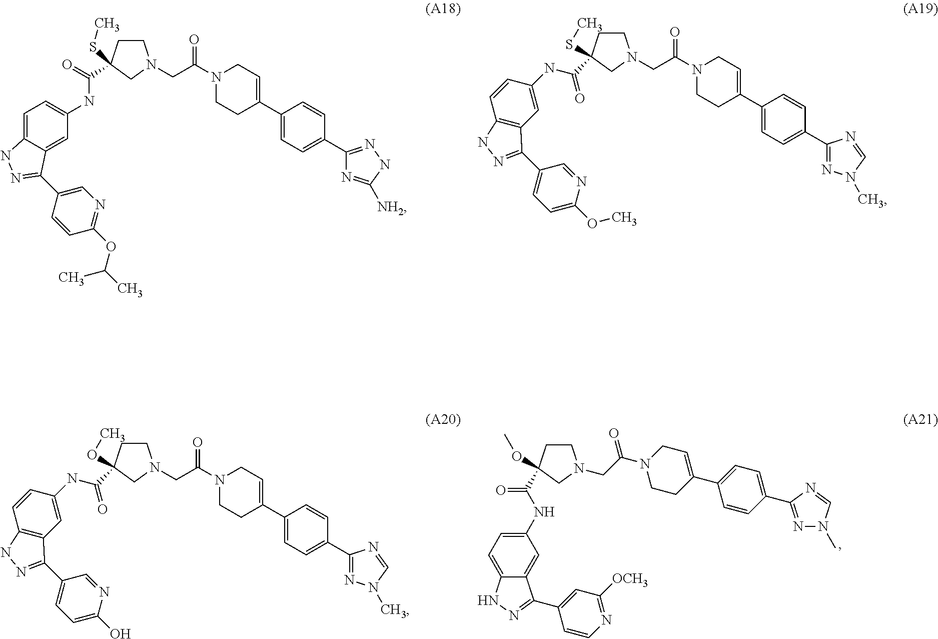

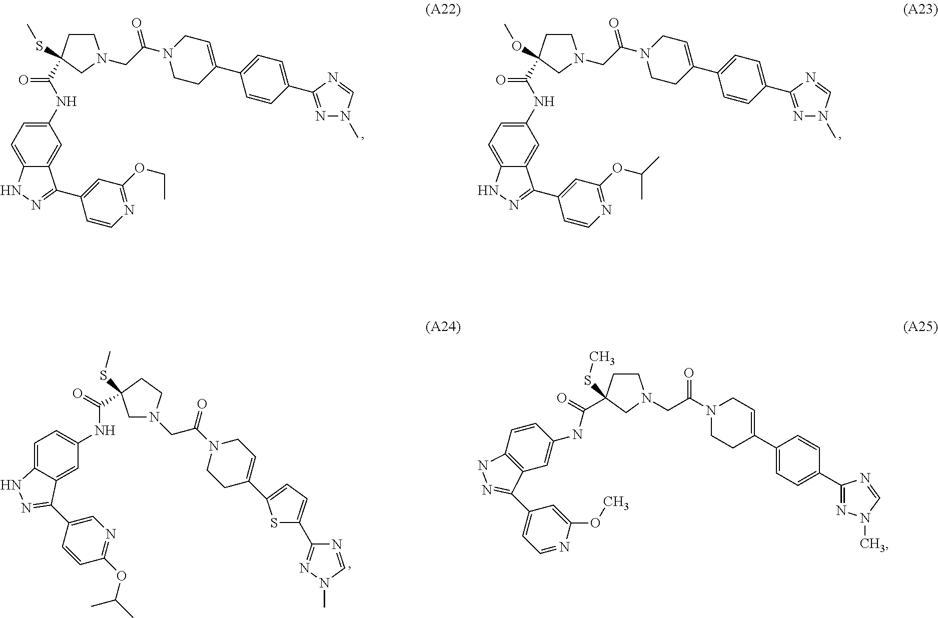

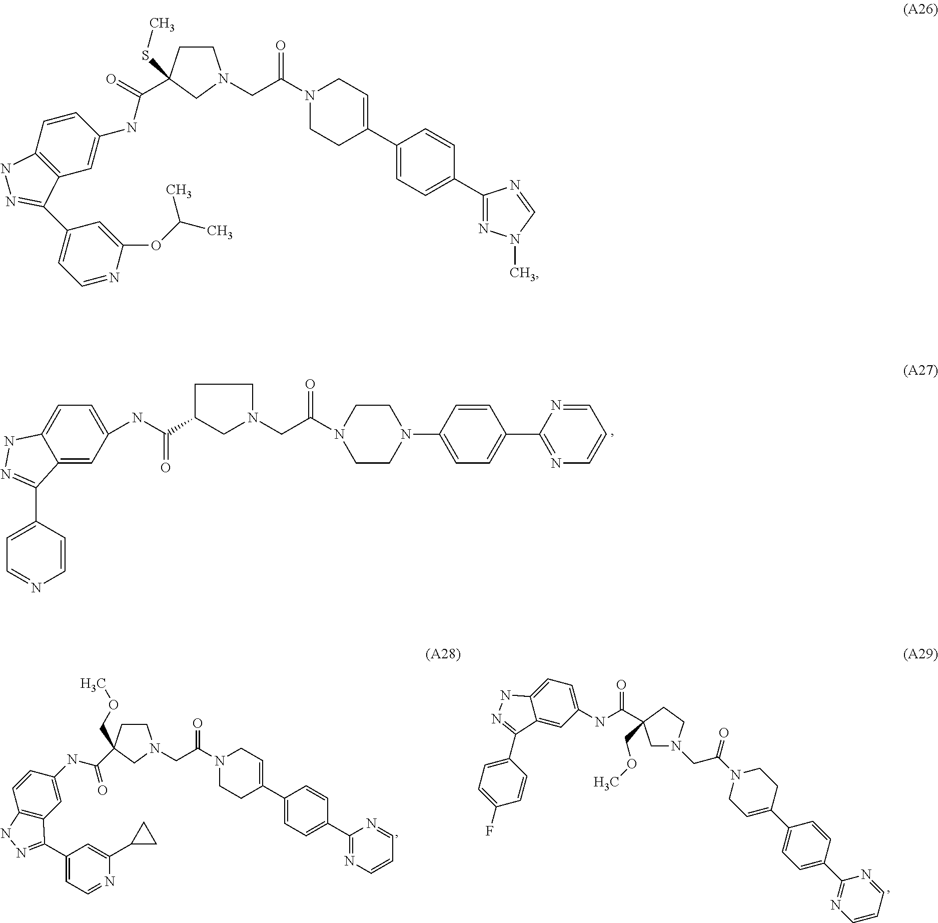

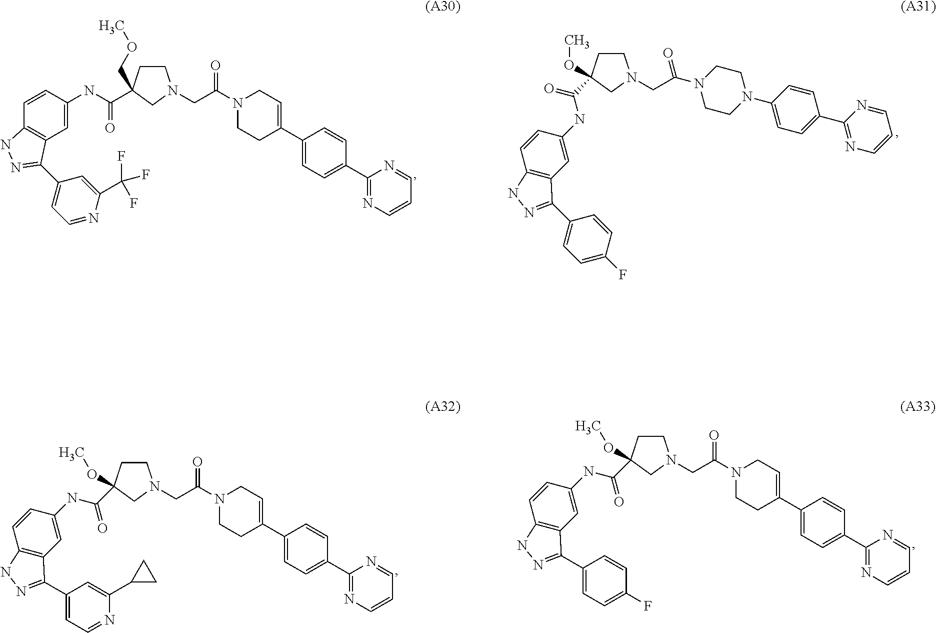

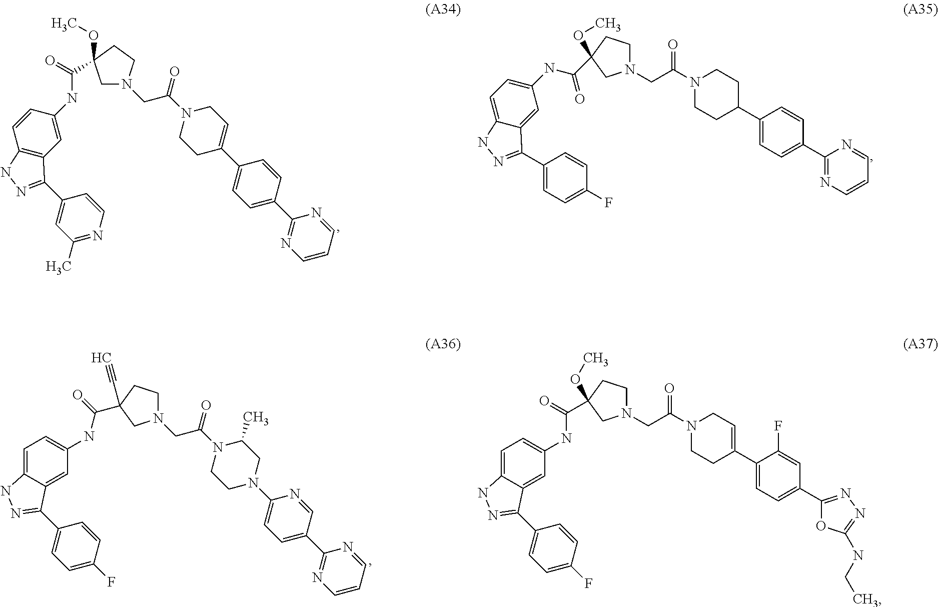

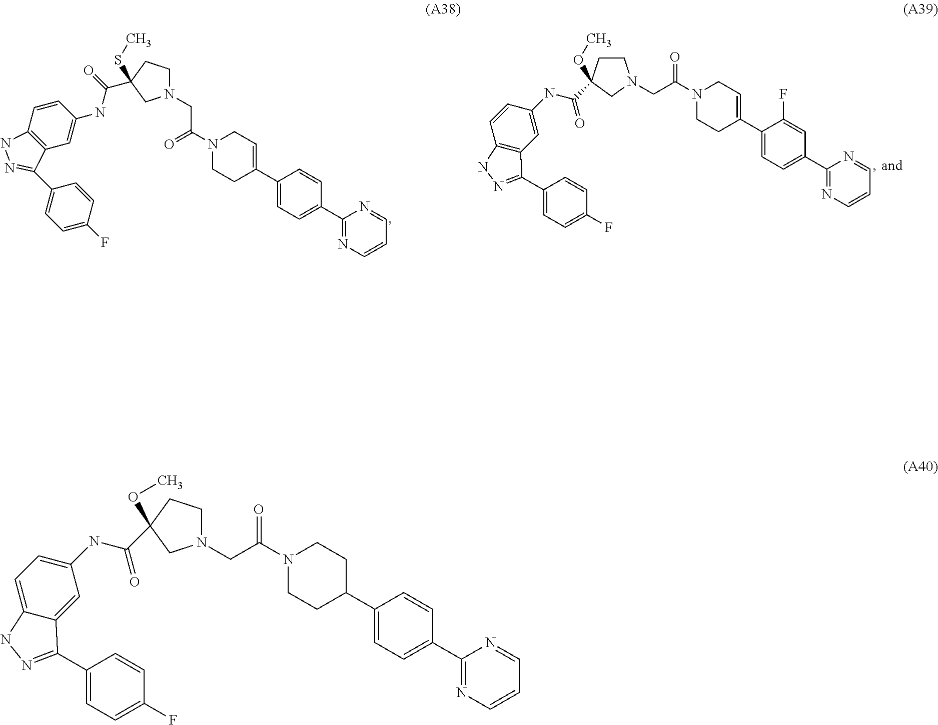

In certain embodiments, the further chemotherapeutic agent is an allogeneic anti-cancer vaccine, an autologous anti-cancer vaccine (e.g., a colorectal cancer cell vaccine or autologous glioma lysate-derived dendritic cell vaccine for glioblastoma multiforme or a cytomegalovirus (CMV) pp65-lysosomal-associated membrane protein (LAMP) mRNA-loaded dendritic cell vaccine or an anti-malignant glioma autologous dendritic cell vaccine wherein autologous dendritic cells (DC) are prepared from autologous PBMC exposed to sargramostim (GM-CSF) and interleukin-4 (IL-4), matured with a cytokine cocktail, and pulsed with synthetic glioma-associated antigen (GAA) peptides or an anti-malignant glioma autologous dendritic cell vaccine wherein autologous dendritic cells are prepared from autologous PBMC exposed to sargramostim (GM-CSF) and interleukin-4 and pulsed with autologous tumor lysates or a glioblastoma tumor lysate pulsed dendritic cell autologous vaccine or irradiated, autologous tumor cells plus sargramostim (GM-CSF) for brain and central nervous system tumor treatment), a dendritic cell vaccine, for example, with dendritic cells (DCs) that have been transduced ex vivo with an adenoviral vector containing the CCL21 gene, an anti-idiotype anti-cancer vaccine or a vector-based anti-cancer vaccine.

The scope of the present invention also includes methods wherein a subject is administered, in association with the regimen set forth herein, one or more antiemetics in order to alleviate symptoms of nausea associated with certain treatments. In an embodiment of the invention, such an antiemetic includes, but is not limited to, casopitant (GlaxoSmithKline), Netupitant (MGI-Helsinn) and other NK-1 receptor antagonists, palonosetron (sold as Aloxi by MGI Pharma), aprepitant (sold as Emend by Merck and Co.; Rahway, N.J.), diphenhydramine (sold as Benadryl® by Pfizer; New York, N.Y.), hydroxyzine (sold as Atarax® by Pfizer; New York, N.Y.), metoclopramide (sold as Reglan® by AH Robins Co,; Richmond, Va.), lorazepam (sold as Ativan® by Wyeth; Madison, N.J.), alprazolam (sold as Xanax® by Pfizer; New York, N.Y.), haloperidol (sold as Haldol® by Ortho-McNeil; Raritan, N.J.), droperidol (Inapsine®), dronabinol (sold as Marinol® by Solvay Pharmaceuticals, Inc.; Marietta, Ga.), dexamethasone (sold as Decadron® by Merck and Co.; Rahway, N.J.), methylprednisolone (sold as Medrol® by Pfizer; New York, N.Y.), prochlorperazine (sold as Compazine® by Glaxosmithkline; Research Triangle Park, N.C.), granisetron (sold as Kytril® by Hoffmann-La Roche Inc.; Nutley, N.J.), ondansetron (sold as Zofran® by Glaxosmithkline; Research Triangle Park, N.C.), dolasetron (sold as Anzemet® by Sanofi-Aventis; New York, N.Y.), tropisetron (sold as Navoban® by Novartis; East Hanover, N.J.).

Other side effects of cancer treatment include red and white blood cell deficiency. Accordingly, the present invention includes methods wherein the subject is administered, in association with the regimen set forth herein, an agent which treats or prevents such a deficiency, such as, e.g., pegfilgrastim, erythropoietin, epoetin alfa or darbepoetin alfa.

### Therapeutic Methods, Dosage and Administration

Methods of the present invention include administration of a therapeutically effective dosage of irinotecan or cyclophosphamide and then an IGF1R antibody or antigen-binding fragment thereof of the invention. In an embodiment of the invention, the administration and dosage of irinotecan or cyclophosphamide is, when possible, done according to the schedule listed in the product information sheet of the approved agents, in the Physicians' Desk Reference 2003 (Physicians' Desk Reference, 57th Ed); Medical Economics Company; ISBN: 1563634457; 57th edition (November 2002), as well as therapeutic protocols well known in the art.

The term colorectal cancer includes all cancers of the colon and/or rectum. For example, the term includes adenocarcinoma of the colon (e.g., mucinous (colloid) adenocarcinoma or signet ring adenocarcinoma). Other types of colorectal cancer included by the term include the following varieties of colon cancer: neuroendocrine, lymphoma, melanoma, squamous cell, sarcoma and carcinoid.

The term colorectal cancer also includes all stages of colorectal cancer; for example, under the Modified Duke Staging System or TNM system (Tumor, Node, Metastasis). The stages associated with these systems are well known by practitioners of ordinary skill in the art.

In an embodiment of the invention, the IGF1R antibody or antigen-binding fragment thereof of the invention is administered to a subject following treatment with irinotecan or cyclophosphamide to treat or prevent colorectal cancer wherein the subject is predisposed to colorectal cancer. For example, in an embodiment of the invention, the patient has familial adenomatous polyposis (FAP), hereditary nonpolyposis colon cancer (HNPCC) (i.e., Lynch I Syndrome or Lynch II Syndrome), inflammatory bowel disease, such as chronic ulcerative colitis (UC) or Crohn's disease, other family cancer syndromes (e.g., Peutz-Jegher Syndromem and Familial Juvenile Polyposis), or adenomatous polyps (e.g., sessile (flat with a broad base and no stalk); tubular (composed of tubular glands extending downward from the outer surface of the polyp); villous (composed of fingerlike epithelial projections extending outward from the surface of the bowel mucosa); pedunculated (attached by a narrow base and a long stalk)). In another embodiment of the invention, the subject is not afflicted with any such predisposition.

HNPCC is, in an embodiment of the invention, mediated by one or more genes such as MLH1, MSH2, PMS1, PMS2, and MSH6 and is characterized by an increased risk of several cancers such as colorectal cancer. HNPCC is inherited as an autosomal dominant trait and includes Lynch I syndrome and Lynch II syndrome. In an embodiment of the invention, Lynch I syndrome is characterized by a familial predisposition to colorectal cancer with right-sided predominance and predominantly early-onset proximal colon carcinomas. In an embodiment of the invention, Lynch syndrome II is characterized by a familial predisposition for other primary cancers in addition to the predisposition for colon cancer.

In an embodiment of the invention, familial adenomatous polyposis (FAP) is an inherited condition in which numerous polyps form mainly in the epithelium of the large intestine. In general, while these polyps start out benign, malignant transformation into colon cancer occurs when not treated.

In an embodiment of the invention, inflammatory bowel disease is the name of a group of disorders that cause the intestines to become inflamed (e.g., red and swollen). Typically, ulcerative colitis and Crohn's disease are classified as inflammatory bowel diseases. Ulcerative colitis is a form of colitis that includes characteristic ulcers or open sores, in the colon. In an embodiment of the invention, Crohn's disease is a chronic inflammatory disease of the intestines. It primarily causes ulcerations (breaks in the lining) of the small and large intestines, but can affect the digestive system anywhere from the mouth to the anus. Crohn's disease is also called granulomatous enteritis or colitis, regional enteritis, ileitis, or terminal ileitis.

In an embodiment of the invention, Peutz-Jegher's (PJ) syndrome is a hereditary condition that results in gastrointestinal polyps and freckles on the skin. The cause of Peutz-Jegher's is an inherited mutation in a gene on chromosome 19, LKB1 or STK 11. The mutation seems to result in a predisposition to benign and cancerous tumors.

In an embodiment of the invention, familial juvenile polyposis (FJP) is an autosomal dominant condition characterized by multiple juvenile polyps of the gastrointestinal (GI) tract. Kindreds have been described in which there is involvement of the colon only, the upper GI tract or both upper and lower GI tracts. FJP is a hamartomatous polyposis syndrome. Although the polyps in PJS are true hamartomata, some may undergo adenomatous change, and these family members are at increased risk for gastrointestinal malignancy. The PJS gene was mapped to chromosome 19p by comparative genomic hybridization and linkage and germline mutations were identified in the serine threonine kinase gene, LKB1.

In an embodiment of the invention, adenomatous polyps (adenomas) of the colon and rectum are benign (noncancerous) growths that may be precursor lesions to colorectal cancer. In general, polyps greater than one centimeter in diameter are associated with a greater risk of cancer. If polyps are not removed, they typically continue to grow and can become cancerous.

The term osteosarcoma includes all types of osteosarcoma, for example, high grade intramedullary osteosarcoma, low grade intramedullary osteosarcoma, parosteal osteosarcoma, periosteal osteosarcoma, high grade surface osteosarcoma, osteosarcoma complicating paget disease, osteosarcoma occurring in irradiated bone and osteosarcoma in the jaw.

The term osteosarcoma also includes all stages of the disease including, for example, stage 1A, stage 1B, stage 2A, stage 2B and stage 3.

The present invention comprises methods for treating or preventing a hyperproliferative disorder comprising administering a therapeutically effective amount or dosage of anti-IGF1R or an antigen-binding fragment thereof following administration of a therapeutically effective amount of irinotecan or cyclophosphamide. The term “therapeutically effective amount” or “therapeutically effective dosage” means that amount or dosage of an antibody or antigen-binding fragment thereof or other therapeutic agent or combination thereof of the invention or composition (e.g., as administered in a method according to the present invention) thereof that will elicit a biological or medical response of a tissue, system, patient, subject or host that is being sought by the administrator (such as a researcher, doctor or veterinarian) which includes any measurable alleviation of the signs, symptoms and/or clinical indicia of the disorder (e.g., tumor growth and/or metastasis) including the prevention, slowing or halting of progression of the disorder to any degree whatsoever. In an embodiment of the invention, the therapeutically effective dosage of a given component of a regimen of the present invention (e.g., irinotecan, cyclophosphamide, IGF1R inhibitor) may be a lower dose than is typically administered when such a component is given alone (e.g., 1, 5, 10 or 15% less).

In an embodiment of the invention, irinotecan is administered at a therapeutically effective dosage of about 125 mg/m², e.g., intravenously, e.g., over 90 min. In an embodiment of the invention, irinotecan is administered in a treatment regimen, as discussed above, on days 1, 8, 15 and 22, followed by about 2 weeks of rest. In an embodiment of the invention, irinotecan is administered at a dosage of about 350 mg/m², e.g., intravenously, e.g., over 90 min. In an embodiment of the invention, the irinotecan is administered about once every 3 weeks.

In an embodiment of the invention, cyclophosphamide is administered at a therapeutically effective dosage of about 40 to 50 mg/kg, e.g., intravenously (e.g., injection or infusion), intramuscularly, intraperitoneally, or intrapleurally, e.g., in divided doses over a period of 2 to 5 days. Another intravenous regimen includes 10 to 15 mg/kg, e.g., given every 7 to 10 days or 3 to 5 mg/kg twice weekly. In an embodiment of the invention, cyclophosphamide is administered in the dosage range of 1 to 5 mg/kg/day for both initial and maintenance dosing.

The anti-IGF1R antibodies and antigen-binding fragments thereof and compositions thereof are, in an embodiment of the invention, administered at a therapeutically effective dosage. For example, in one embodiment of the invention, a “therapeutically effective dosage” of any anti-IGF1R antibody or antigen-binding fragment thereof of the present invention is between about 0.3 and 20 mg/kg of body weight (e.g., about 0.3 mg/kg of body weight, about 0.6 mg/kg of body weight, about 0.9 mg/kg of body weight, about 1 mg/kg of body weight, about 2 mg/kg of body weight, about 3 mg/kg of body weight, about 4 mg/kg of body weight, about 5 mg/kg of body weight, about 6 mg/kg of body weight, about 7 mg/kg of body weight, about 8 mg/kg of body weight, about 9 mg/kg of body weight, about 10 mg/kg of body weight, about 11 mg/kg of body weight, about 12 mg/kg of body weight, about 13 mg/kg of body weight, about 14 mg/kg of body weight, about 15 mg/kg of body weight, about 16 mg/kg of body weight, about 17 mg/kg of body weight, about 18 mg/kg of body weight, about 19 mg/kg of body weight, about 20 mg/kg of body weight), about once per week to about once every 6 weeks (e.g., about once every 1 week, once every 2 weeks, once every 3 weeks, once every 4 weeks, once every 5 weeks or once every 6 weeks).

Dosage regimens may be adjusted to provide the optimum desired response (e.g., a therapeutic response). For example, a single dose may be administered or several divided doses may be administered over time or the dose may be proportionally reduced or increased as indicated by the exigencies or the particular circumstances or requirements of the therapeutic situation. For example, dosage may be determined or adjusted, by a practitioner of ordinary skill in the art (e.g., physician or veterinarian) according to the patient's age, weight, height, past medical history, present medications and the potential for cross-reaction, allergies, sensitivities and adverse side-effects. For example, the physician or veterinarian could start doses of the antibody or antigen-binding fragment of the invention or composition thereof at levels lower than that required in order to achieve the desired therapeutic effect and gradually increase the dosage until the desired effect is achieved. The effectiveness of a given dose or treatment regimen of an antibody or combination of the invention can be determined, for example, by determining whether a tumor being treated in the subject shrinks or ceases to grow. The size and progress of a tumor can be easily determined, for example, by X-ray, magnetic resonance imaging (MRI) or visually in a surgical procedure. In general, tumor size and proliferation can be measured by use of a thymidine PET scan (see e.g., Wells et al., Clin. Oncol. 8: 7-14 (1996)). Generally, the thymidine PET scan includes the injection of a radioactive tracer, such as [2-¹¹C]-thymidine, followed by a PET scan of the patient's body (Vander Borght et al., Gastroenterology 101: 794-799, 1991; Vander Borght et al., J. Radiat. Appl. Instrum. Part A, 42: 103-104 (1991)). Other tracers that can be used include [¹⁸F]-FDG (18-fluorodeoxyglucose), [¹²⁴I]IUdR (5-[124I]iodo-2′-deoxyuridine), [⁷⁶Br]BrdUrd (Bromodeoxyuridine), [¹⁸F]FLT (3′-deoxy-3′ fluorothymidine) or [¹¹C]FMAU (2′-fluoro-5-methyl-1-β-D-arabinofuranosyluracil).

For example, colorectal or colon cancer progress can be monitored, by the physician, by a variety of methods, and the dosing regimen can be altered accordingly. Methods by which to monitor colorectal or colon cancer include CT scan, MRI scan, chest X-ray, PET scan, fecal occult blood tests (FOBTs), flexible proctosigmoidoscopy, total colonoscopy, and barium enema.

For example, osteosarcoma progress can be monitored, by the physician, by methods including, e.g., an X-ray of the affected area, CT (computerised tomography) or MRI (magnetic resonance imaging) scan of the affected area, blood analysis (e.g., to determine LDH (lactate dehydrogenase) or ALP (alkaline phosphatase) levels—higher LDH or ALP is associated with bone activity and osteosarcoma), CT or MRI scan of the chest to see if the cancer has spread to the lungs, open biopsy (at time of surgery for diagnosis), needle biopsy of the affected bone, and a bone scan to see if the cancer has spread to other bones (e.g., using technetium-99 or thallium-201 as a tracer).

The term “subject” or “patient” includes any mammal (e.g., primate, dog, horse, rat, mouse, cat, rabbit) including a human. In an embodiment of the invention, a “subject” or “patient” is an adult human (e.g., 18 years or older) or a human child (e.g., under 18 years of age, for example, less than 1, 1, 2, 3, 4, 5, 6, 7, 8, 9 or 10 years of age); or a female or a male. For example, in the context of a method of treatment, a subject or a patient is a mammal, such as a human, with a hyperproliferative disorder who is in need of a treatment which is set forth herein.

### Pharmaceutical Compositions

Methods for treating or preventing hyperproliferative disorders by administering pharmaceutical compositions comprising irinotecan or cyclophosphamide; and an anti-IGF1R antibody or antigen-binding fragment thereof of the invention, wherein any of which is combined with a pharmaceutically acceptable carrier, are also within the scope of the present invention (e.g., in a single composition or separately in a kit). The pharmaceutical compositions may be prepared by any methods well known in the art of pharmacy; see, e.g., Gilman, et al., (eds.) (1990), The Pharmacological Bases of Therapeutics, 8th Ed., Pergamon Press; A. Gennaro (ed.), Remington's Pharmaceutical Sciences, 18th Edition, (1990), Mack Publishing Co., Easton, Pa.; Avis, et al., (eds.) (1993) Pharmaceutical Dosage Forms: Parenteral Medications Dekker, New York; Lieberman, et al., (eds.) (1990) Pharmaceutical Dosage Forms: Tablets Dekker, New York; and Lieberman, et al., (eds.) (1990), Pharmaceutical Dosage Forms: Disperse Systems Dekker, New York.

In an embodiment of the invention, the antibody or antigen-binding fragment thereof is administered to a subject as part of a pharmaceutical composition comprising sodium acetate (e.g., Trihydrate USP) at 2.30 mg/ml; glacial acetic acid (e.g., USP/Ph. Eur) at 0.18 mg/ml; sucrose (e.g., extra pure NF, Ph. Eur, BP) at 70.0 mg/ml; anti-IGF1R antibody or an antigen-binding fragment thereof at 20.0 mg/ml and water, for example, sterile water (e.g., for injection USP/Ph. Eur); at a pH of about 5.5 to about 6.0 (e.g., 5.5, 5.6, 5.7, 5.8, 5.9, 6.0). If a lyophilized powder thereof (also part of the present invention) is prepared, water is added to reconstitute the composition for use (see e.g., international application publication no. WO2006/138315).

A pharmaceutical composition containing an antibody or antigen-binding fragment thereof of the invention, which is optionally in association with a further chemotherapeutic agent, can be prepared using conventional pharmaceutically acceptable excipients and additives and conventional techniques. Such pharmaceutically acceptable excipients and additives include non-toxic compatible fillers, binders, disintegrants, buffers, preservatives, anti-oxidants, lubricants, flavorings, thickeners, coloring agents, emulsifiers and the like. All routes of administration are contemplated including, but not limited to, parenteral (e.g., subcutaneous, intravenous, intraperitoneal, intramuscular, topical, intra-peritoneal, inhalation, intra-cranial) and non-parenteral (e.g., oral, transdermal, intranasal, intraocular, sublingual, rectal and topical).

Injectables can be prepared in conventional forms, either as liquid solutions or suspensions, solid forms suitable for solution or suspension in liquid prior to injection, or as emulsions. The injectables, solutions and emulsions can also contain one or more excipients. Excipients include, for example, water, saline, dextrose, glycerol or ethanol. In addition, if desired, the pharmaceutical compositions to be administered may also contain minor amounts of non-toxic auxiliary substances such as wetting or emulsifying agents, pH buffering agents, stabilizers, solubility enhancers, or other such agents, such as for example, sodium acetate, sorbitan monolaurate, triethanolamine oleate or cyclodextrins.

In an embodiment of the invention, pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents or other pharmaceutically acceptable substances.

Examples of aqueous vehicles include sodium chloride injection, Ringers Injection, Isotonic Dextrose Injection, Sterile Water Injection, Dextrose or Lactated Ringers Injection. Nonaqueous parenteral vehicles include fixed oils of vegetable origin, cottonseed oil, corn oil, sesame oil or peanut oil. Antimicrobial agents in bacteriostatic or fungistatic concentrations may be added to parenteral preparations packaged in multiple-dose containers which include phenols or cresols, mercurials, benzyl alcohol, chlorobutanol, methyl or propyl p-hydroxybenzoic acid esters, thimerosal, benzalkonium chloride or benzethonium chloride. Isotonic agents include sodium chloride or dextrose. Buffers include phosphate or citrate. Antioxidants include sodium bisulfate, Local anesthetics include procaine hydrochloride. Suspending and dispersing agents include sodium carboxymethylcelluose, hydroxypropyl methylcellulose or polyvinylpyrrolidone. Emulsifying agents include Polysorbate 80 (TWEEN-80). A sequestering or chelating agent of metal ions includes EDTA (ethylenediaminetetraacetic acid) or EGTA (ethylene glycol tetraacetic acid). Pharmaceutical carriers may also include ethyl alcohol, polyethylene glycol or propylene glycol for water miscible vehicles; or sodium hydroxide, hydrochloric acid, citric acid or lactic acid for pH adjustment.

In an embodiment of the invention, preparations for parenteral administration can include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

The concentration of the antibody or antigen-binding fragment thereof of the invention can be adjusted so that an injection provides an effective amount to produce the desired pharmacological effect. As discussed herein, the exact dose depends, in part, on the age, weight and condition of the patient or animal as is known in the art.

In an embodiment of the invention, unit-dose parenteral preparations are packaged in an ampoule, a vial or a syringe with a needle. All preparations for parenteral administration must be sterile, as is known and practiced in the art.

In an embodiment of the invention, a sterile, lyophilized powder is prepared by dissolving the antibody or antigen-binding fragment thereof or a pharmaceutical composition thereof, in a suitable solvent. The solvent may contain an excipient which improves the stability or other pharmacological components of the powder or reconstituted solution, prepared from the powder. Excipients that may be used include, but are not limited to, dextrose, sorbital, fructose, corn syrup, xylitol, glycerin, glucose, sucrose or other suitable agent. The solvent may also contain a buffer, such as citrate, sodium or potassium phosphate or other such buffer known to those of skill in the art at, in one embodiment, about neutral pH. Subsequent sterile filtration of the solution followed by lyophilization under standard conditions known to those of skill in the art provides a desirable formulation. In one embodiment, the resulting solution will be apportioned into vials for lyophilization. Each vial can contain a single dosage or multiple dosages of the anti-IGF1R antibody or antigen-binding fragment thereof or composition thereof. Overfilling vials with a small amount above that needed for a dose or set of doses (e.g., about 10%) is acceptable so as to facilitate accurate sample withdrawal and accurate dosing. The lyophilized powder can be stored under appropriate conditions, such as at about 4° C. to room temperature.

Reconstitution of a lyophilized powder with water for injection provides a formulation for use in parenteral administration. In an embodiment of the invention, for reconstitution, the lyophilized powder is added to sterile water or other liquid suitable carrier. The precise amount depends upon the selected therapy being given. Such amounts can be empirically determined.

Implantation of a slow-release or sustained-release system, such that a constant level of dosage is maintained, is also contemplated herein. Briefly, an active agent is dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, or ethylene/vinyloxyethanol copolymer, that is insoluble in body fluids. The compound diffuses through the outer polymeric membrane in a release rate controlling step. The percentage of active compound contained in such parenteral compositions is highly dependent on the specific nature thereof as well as the activity of the antibody or antigen-binding fragment and the needs of the subject.

Agents set forth herein can be formulated into a sustained release formulation including liposomal formulations such as unilamellar vesicular (ULV) and multilamellar vesicular (MLV) liposomes and DepoFoam™ particles (Kim et al., Biochim. Biophys. Acta (1983) 728(3):339-348; Kim, Methods Neurosci. (1994) 21: 118-131; Kim et al., Anesthesiology (1996) 85(2): 331-338; Katre et al., J. Pharm. Sci. (1998) 87(11): 1341-1346). A feature of the DepoFoam system is that, inside each DepoFoam particle, discontinuous internal aqueous chambers, bounded by a continuous, non-concentric network of lipid membranes render a higher aqueous volume-to-lipid ratio and much larger particle diameters compared with MLV.

In an embodiment of the invention, irinotecan is in an aqueous solution, for example, wherein each milliliter of solution contains about 20 mg of irinotecan hydrochloride (on the basis of the trihydrate salt), about 45 mg of sorbitol NF powder, and about 0.9 mg of lactic acid, USP; wherein the pH of the solution has been adjusted to about 3.5 (range, 3.0 to 3.8) with sodium hydroxide or hydrochloric acid. In an embodiment of the invention, the aqueous irinotecan solution is, when prepared for administration, diluted with 5% Dextrose Injection, USP (D5W), or 0.9% Sodium Chloride Injection, USP, for example, prior to intravenous infusion.

In an embodiment of the invention, cyclophosphamide, for injection, is cyclophosphamide monohydrate. For example, in an embodiment of the invention, cyclophosphamide for injection (e.g., intravenously, intramuscularly, intraperitoneally, or intrapleurally) or infusion can be reconstituted, from a dry powder form, by adding 0.9% sterile sodium chloride solution.

In an embodiment of the invention, cyclophosphamide for oral administration (e.g., a tablet) is cyclophosphamide anhydrous, for example, wherein a cyclophosphamide tablet comprises the inactive ingredients: acacia, FD&C Blue No. 1, D&C Yellow No. 10 Aluminum Lake, lactose, magnesium stearate, starch, stearic acid and talc.

### EXAMPLES

The following information is provided for more clearly describing the present invention and should not be construed to limit the present invention. Any and all of the compositions and methods described below fall within the scope of the present invention.

### Example 1

### Sequential Administration of Irinotecan, then Anti-IGF1R Antibody for the Treatment of Human Colorectal Cancer in Xenograft Mice

This example demonstrates that administration of anti-IGF1R antibody (LCF/HCA) (3 doses, 2×/wk) following 3 doses of irinotecan (2×/wk) showed better efficacy than irinotecan alone (p=0.08 and 0.02 for 0.1 mg and 0.5 mg anti-IGF1R dosage, respectively). Administration of anti-IGF1R (3 doses, 2×/wk) following 3 doses of irinotecan (2×/wk) was better than simultaneous co-administration of the two agents (same total dosage) (p=0.07 for both 0.1 and 0.5 mg anti-IGF1R combination groups).

Five million WiDr colon cancer cells in a 1:1 mix with regular Matrigel (BD Biosciences; San Jose, Calif.) were inoculated in 150 nude mice subcutaneously on the right flank. When tumors reached about 95 mm³ in 10 days, 100 mice were sorted into 10 groups of 10 mice each. Dosing was started the same day as they were grouped. Tumor size and body weight were measured twice weekly.

For these experiments, anti-IGF1R antibody (LCF/HCA (γ1, κ)) stock (34.06 mg/ml) was used and diluted in 5 mM NaAc, pH 5.5. Irinotecan/Camptosar (clinical grade from Pharmacia; New York, N.Y.) was diluted with 5 ml of Saline (0.9% sodium chloride) for 10 mg/ml for a 100 mpk irinotecan solution.

Four million HT29 colon cells in a 1:1 mix with regular Matrigel (BD Biosciences; San Jose, Calif.) were inoculated in 100 nude mice subcutaneously on the right flank. When tumors reached about 100 mm³ in 7 days, 64 mice were sorted into 8 groups of 8 mice each. Dosing was started the day after they were grouped. Tumor size and body weight were measured twice weekly.

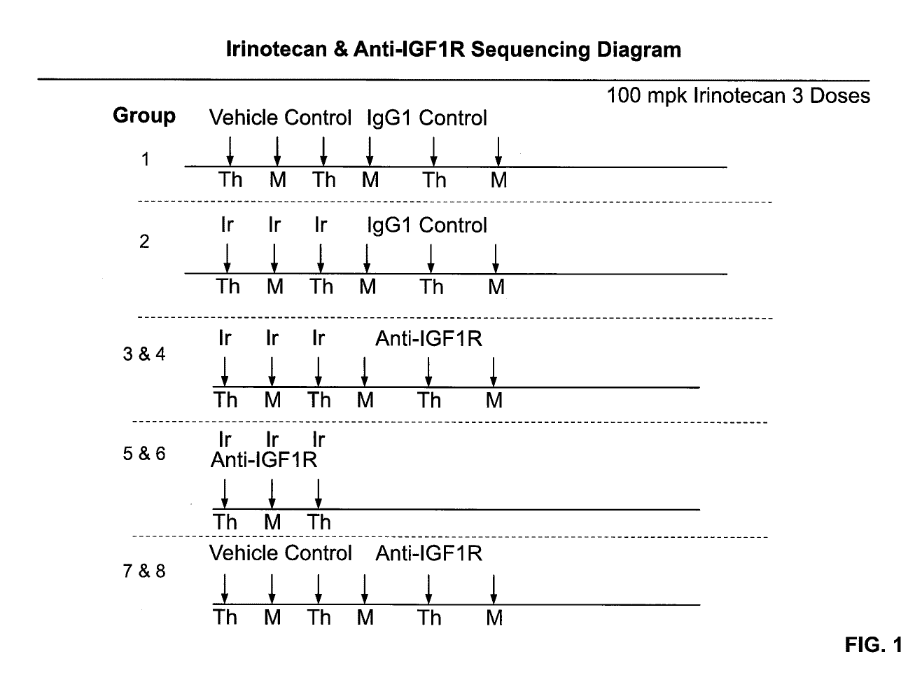

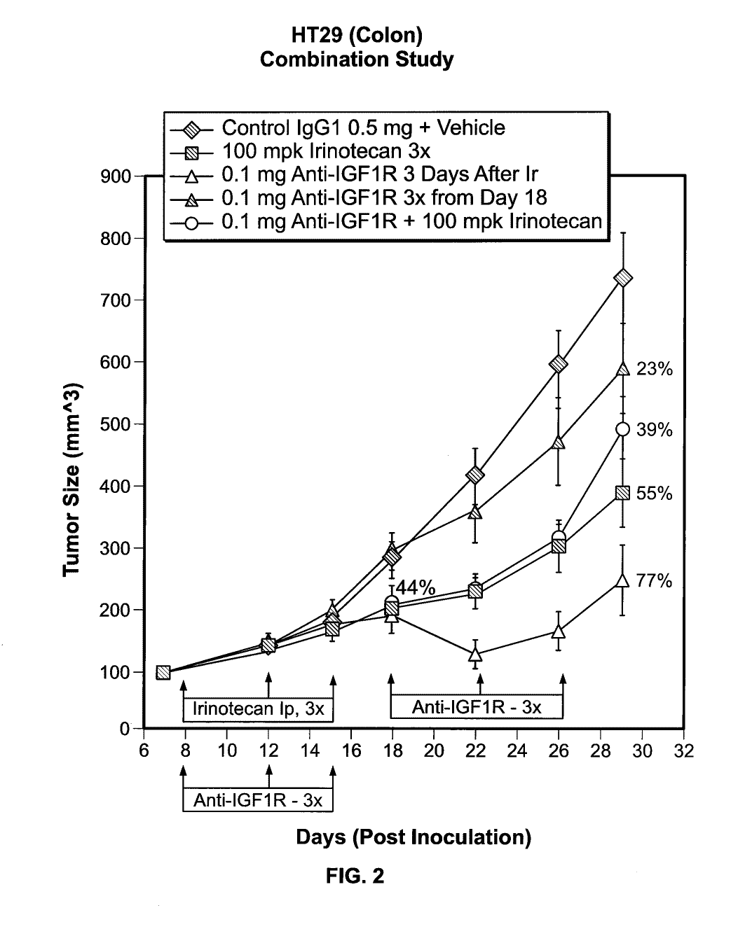

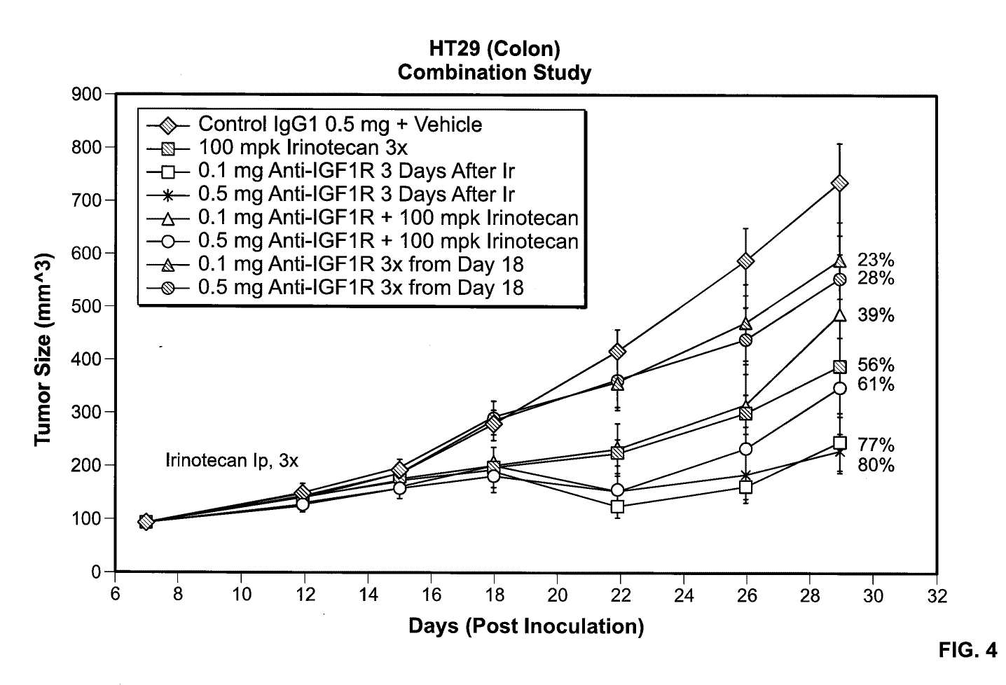

The design of this experiment was as shown in FIG. 1. FIGS. 2-4 show the tumor size in the groups analyzed over time for the mice with HT29 colon cancer cells treated with irinotecan. FIG. 2 shows that the lowest level of tumor growth occurred in the group treated with anti-IGF1R (0.1 mg) after irinotecan (100 mpk (mg/kg body weight)) (open triangles). This group exhibited 77% tumor growth inhibition. By contrast, coadministration of the anti-IGF1R antibody with irinotecan exhibited only 39% tumor growth inhibition (open circles).

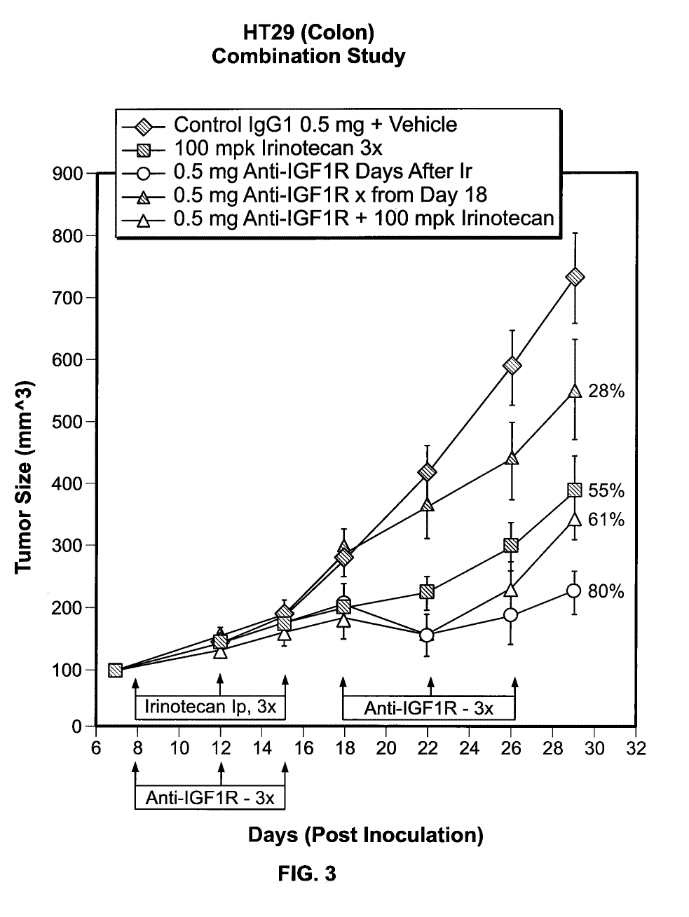

Similarly, the data in FIG. 3 indicates a similar result. The lowest level of tumor growth occurred in the group treated with anti-IGF1R (0.5 mg) after irinotecan (100 mpk (mg/kg body weight)) (open circles). This group exhibited 80% tumor growth inhibition. By contrast, coadministration of the anti-IGF1R antibody with irinotecan exhibited only 61% tumor growth inhibition (open triangles).

### Example 2

### Sequential Administration of Cyclophosphamide, then Anti-IGF1R Antibody for the Treatment of Human Ostreosarcoma in Xenograft Mice

This example demonstrates that the combination of anti-IGF1R antibody with cyclophosphamide was more effective at inhibiting tumor growth than either single agent alone. First, giving cyclophosphamide, then, followed 2 days later, with anti-IGF1R, was more effective at tumor growth inhibition than the reverse—administering anti-IGF1R first followed, 2 days later, with cyclophosphamide.

Four million SJSA-1 osteosarcoma cells with 1:1 mix of regular Matrigel (BD Biosciences; San Jose, Calif.) were inoculated into 142 nude mice subcutaneously on the right flank. When tumors reach about 250 mm³ in 22 days, 90 mice were sorted into 9 groups of 10 mice each. Dosing was started the day of grouping. Tumor size and body weight was measured twice weekly.

Anti-IGF1R antibody (LCF/HCA (γ1, κ)) stock (15 mg/ml) was used and diluted in 20 mM NaAc, 2.3% sucrose, pH 5.5.

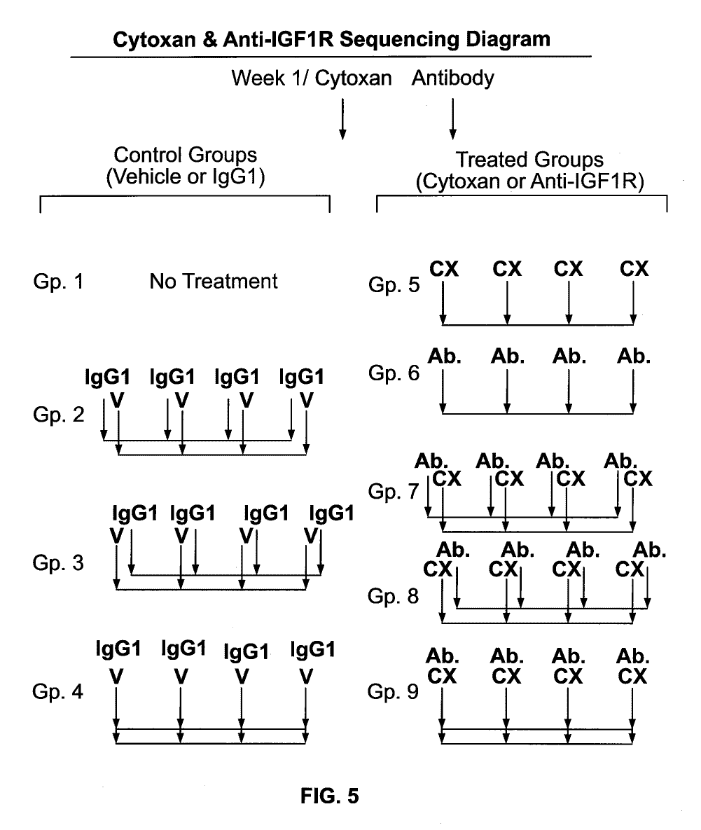

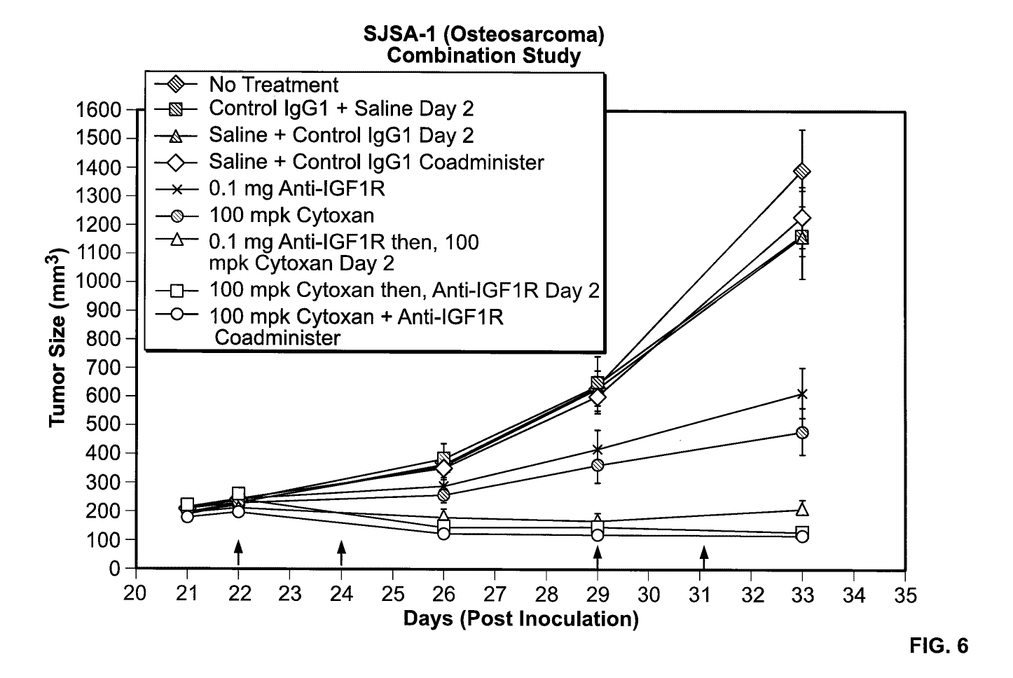

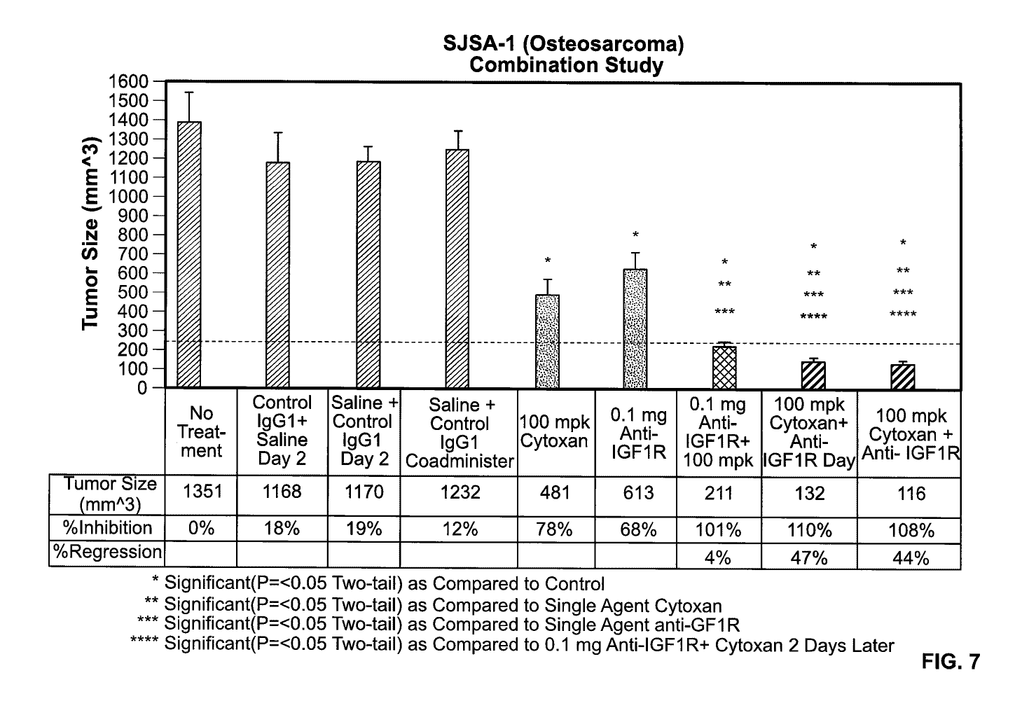

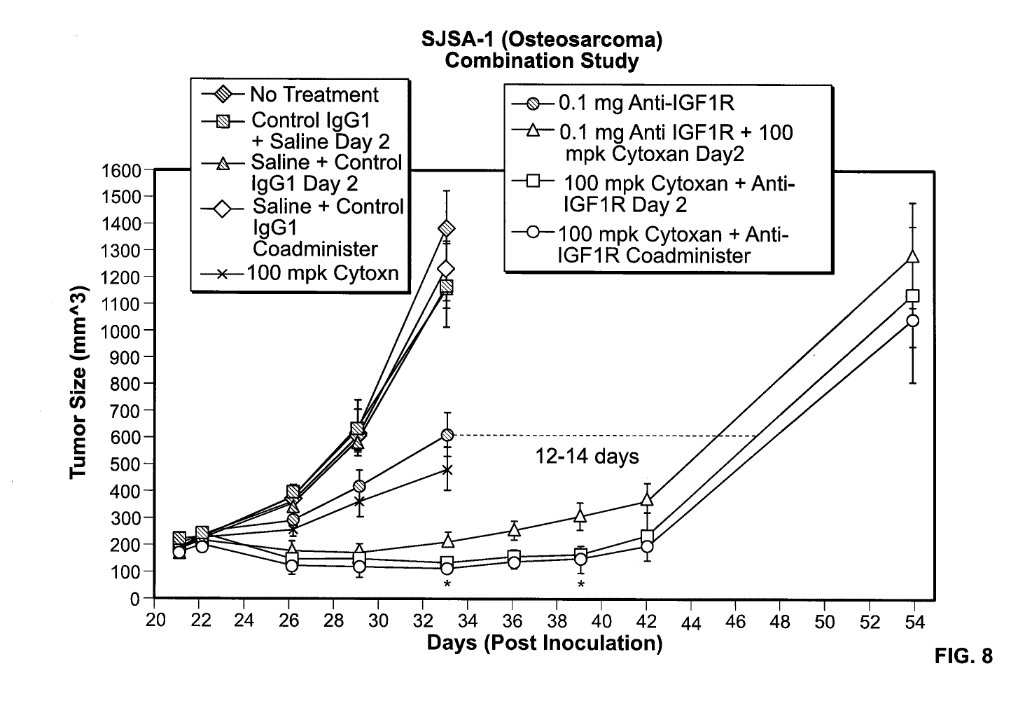

The design of this experiment was as shown in FIG. 5. FIGS. 6-8 show the tumor volume in the groups analyzed over time. FIG. 6 demonstrates that administration of cyclyphosphamide, then the anti-IGF1R antibody caused the greatest level of tumor growth inhibition (open triangles); an inhibition level greater than administration of the antibody, then cyclophosphamide (open square) or coadministration of both agents simultaneously (open triangle). A bar graphical representation of the results in FIG. 6 are set forth in FIG. 7. A followup study was conducted to follow tumor growth following cessation of treatment, after day 38. Tumor volume increased following cessation in the groups that were monitored (FIG. 8).

The present invention is not to be limited in scope by the specific embodiments described herein. Indeed, the scope of the present invention includes embodiments specifically set forth herein and other embodiments not specifically set forth herein; the embodiments specifically set forth herein are not necessarily intended to be exhaustive. Various modifications of the invention in addition to those described herein will become apparent to those skilled in the art from the foregoing description. Such modifications are intended to fall within the scope of the claims.

Patents, patent applications, publications, product descriptions, and protocols are cited throughout this application, the disclosures of which are incorporated herein by reference in their entireties for all purposes.

We claim:

1. A method for treating or preventing a hyperproliferative disorder mediated by elevated expression or activity of insulin-like growth factor I receptor or elevated expression of IGF-1 or elevated expression of IGF-II, in a subject, comprising first administering a therapeutically effective amount of a cytotoxic anti-cancer chemotherapeutic agent to the subject, then administering a therapeutically effective amount of an IGF1R inhibitor to the subject.

2. A method for treating or preventing a hyperproliferative disorder mediated by elevated expression or activity of insulin-like growth factor I receptor or elevated expression of IGF-1 or elevated expression of IGF-II, in a subject, comprising first administering a therapeutically effective amount of cyclophosphamide or irinotecan to the subject, then administering a therapeutically effective amount of an IGF1R inhibitor to the subject.

3. The method of claim 1 wherein the IGF1R inhibitor is an isolated antibody or antigen-binding fragment thereof comprising one or more members selected from the group consisting of:
(a) CDR-L1, CDR-L2 and CDR-L3 of the variable region of 15H12/19D12 light chain C, 15H12/19D12 light chain D, 15H12/19D12 light chain E or 15H12/19D12 light chain F; or
(b) CDR-H1, CDR-H2 and CDR-H3 of the variable region of 15H12/19D12 heavy chain A or 15H12/19D12 heavy chain b; or both.

4. The method of claim 1 wherein:
CDR-L1 comprises the amino acid sequence:
(SEQ ID NO: 1)
Arg Ala Ser Gln Ser Ile Gly Ser Ser Leu His;
CDR-L2 comprises the amino acid sequence:

| | |
|---|---|
| Tyr Ala Ser Gln Ser Leu Ser; | (SEQ ID NO: 2) |

CDR-L3 comprises the amino acid sequence:
(SEQ ID NO: 3)
His Gln Ser Ser Arg Leu Pro His Thr;
CDR-H1 comprises the amino acid sequence:
(SEQ ID NO: 4)
Ser Phe Ala Met His
or
(SEQ ID NO: 5)
Gly Phe Thr Phe Ser Ser Phe Ala Met His;
CDR-H2 comprises the amino acid sequence:
(SEQ ID NO: 6)
Val Ile Asp Thr Arg Gly Ala Thr Tyr Tyr Ala Asp
Ser Val Lys Gly;
and/or
CDR-H3 comprises the amino acid sequence:
(SEQ ID NO: 7)
Leu Gly Asn Phe Tyr Tyr Gly Met Asp Val.

5. The method of claim 1 wherein the antibody or fragment is in a pharmaceutical composition which comprises a pharmaceutically acceptable carrier.

6. The method of claim 1 wherein the antibody or fragment comprises a light chain variable region comprising amino acids 20-128 of SEQ ID NO: 9, 11, 13 or 15 and a heavy chain variable region comprising amino acids 20-137 of SEQ ID NO: 17 or 19.

7. The method of claim 6 wherein said antibody or antigen-binding fragment is an antibody which is a monoclonal antibody.

8. The method of claim 7 wherein the monoclonal antibody is in a pharmaceutical composition which comprises a pharmaceutically acceptable carrier.

9. The method of claim 1 wherein said antibody or fragment is an antibody and the antibody is a labeled antibody, bivalent antibody, a polyclonal antibody, a bispecific antibody, a chimeric antibody, a recombinant antibody, an anti-idiotypic antibody, a humanized antibody or a bispecific antibody.

10. The method of claim 1 wherein the antibody or fragment is a fragment and the fragment is a camelized single domain antibody, a diabody, an scfv, an scfv dimer, a dsfv, a (dsfv)2, a dsFv-dsfv′, a bispecific ds diabody, a nanobody, an Fv, an Fab, an Fab′, an F(ab′)₂, or a domain antibody.

11. The method of claim 1 wherein the antibody or fragment is linked to a constant region.

12. The method of claim 11 wherein the constant region is a κ light chain, γ1 heavy chain, γ2 heavy chain, γ3 heavy chain or γ4 heavy chain.

13. The method of claim 1 wherein the subject is administered a further chemotherapeutic agent or an anti-cancer therapeutic procedure.

14. The method of claim 13 wherein the anti-cancer therapeutic procedure is anti-cancer radiation therapy or surgical tumorectomy.

15. The method of claim 13 wherein the further chemotherapeutic agent is an anti-cancer chemotherapeutic agent.

16. The method of claim 1 comprising first administering a therapeutically effective amount of irinotecan to the subject, then administering a therapeutically effective amount of an IGF1R inhibitor to the subject.

17. The method of claim 1 comprising first administering a therapeutically effective amount of cyclophosphamide to the subject, then administering a therapeutically effective amount of an IGF1R inhibitor to the subject.

18. The method of claim 1 wherein the subject is a human.

19. The method of claim 18 wherein the subject is further administered an additional chemotherapeutic agent.

20. The method of claim 1 wherein the disorder is colorectal cancer.

21. The method of claim 1 wherein the disorder is osteosarcoma.

22. The method of claim 1 wherein the disorder is a member selected from the group consisting of:
osteosarcoma, rhabdomyosarcoma, neuroblastoma, any pediatric cancer, kidney cancer, leukemia, renal transitional cell cancer, bladder cancer, Wilm's cancer, ovarian cancer, pancreatic cancer, benign prostatic hyperplasia, breast cancer, prostate cancer, bone cancer, lung cancer, gastric cancer, colorectal cancer, cervical cancer, synovial sarcoma, diarrhea associated with metastatic carcinoid, vasoactive intestinal peptide secreting tumors, psoriasis, smooth muscle restenosis of blood vessels and inappropriate microvascular proliferation, head and neck cancer, squamous cell carcinoma, multiple myeloma, solitary plasmacytoma, renal cell cancer, retinoblastoma, germ cell tumors, hepatoblastoma, hepatocellular carcinoma, melanoma, rhabdoid tumor of the kidney, Ewing Sarcoma, chondrosarcoma, haemotological malignancy, chronic lymphoblastic leukemia, chronic myelomonocytic leukemia, acute lymphoblastic leukemia, acute lymphocytic leukemia, acute myelogenous leukemia, acute myeloblastic leukemia, chronic myeloblastic leukemia, Hodgekin's disease, non-Hodgekin's lymphoma, chronic lymphocytic leukemia, chronic myelogenous leukemia, myelodysplastic syndrome, hairy cell leukemia, mast cell leukemia, mast cell neoplasm, follicular lymphoma, diffuse large cell lymphoma, mantle cell lymphoma, Burkitt Lymphoma, mycosis fungoides, seary syndrome, cutaneous T-cell lymphoma, chronic myeloproliferative disorders, a cental nervous system tumor, brain cancer, glioblastoma, non-glioblastoma brain cancer, meningioma, pituitary adenoma, vestibular schwannoma, a primitive neuroectodermal tumor, medulloblastoma, astrocytoma, anaplastic astrocytoma, oligodendroglioma, ependymoma and choroid plexus papilloma, a myeloproliferative disorder, polycythemia vera, thrombocythemia, idiopathic myelfibrosis, soft tissue sarcoma, thyroid cancer, endometrial cancer, carcinoid cancer, germ cell tumors, liver cancer.

23. The method of claim 13 wherein the further chemotherapeutic agent is one or more members selected from the group consisting of:
everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an EGFR TK inhibitor, an aurora kinase inhibitor, a PIK-1 modulator, a Bcl-2 inhibitor, an HDAC inhibitor, a c-MET inhibitor, a PARP inhibitor, a Cdk inhibitor, an EGFR TK inhibitor, an IGFR-TK inhibitor, an anti-HGF antibody, a PI3 kinase inhibitors, an AKT inhibitor, a JAK/STAT inhibitor, a checkpoint-1 or 2 inhibitor, a focal adhesion kinase inhibitor, a Map kinase kinase (mek) inhibitor, a VEGF trap antibody, pemetrexed, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR, KRX-0402, lucanthone, LY 317615, neuradiab, vitespan, Rta 744, Sdx 102, talampanel, atrasentan, Xr 311, romidepsin, ADS-100380, sunitinib, 5-fluorouracil, leucovorin, 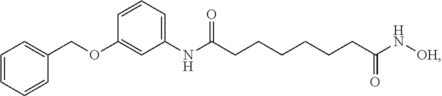
CG-781, CG-1521, 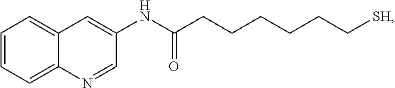
SB-556629, chlamydocin, JNJ-16241199, 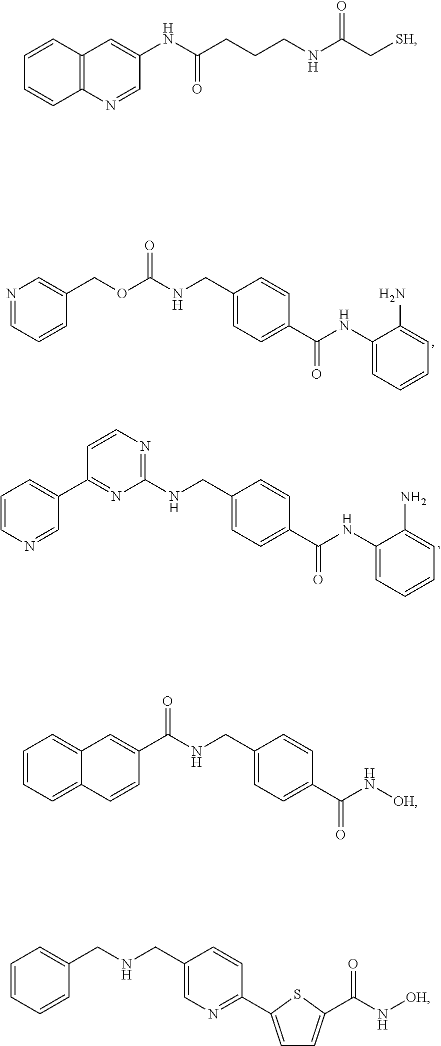
vorinostat, etoposide, gemcitabine, doxorubicin, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin, PEG-labeled irinotecan, tamoxifen, toremifene citrate, anastrazole, exemestane, letrozole, DES (diethylstilbestrol), estradiol, estrogen, conjugated estrogen, bevacizumab, IMC-1C11, CHIR-258, 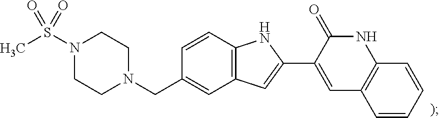
3-[5-(methylsulfonylpiperadinemethyl)indolyl]-quinolone, vatalanib, AG-013736, AVE-0005, the acetate salt of [D-Ser(Bu t) 6, Azgly 10] (pyro-Glu-His-Trp-Ser-Tyr-D-Ser(Bu t)-Leu-Arg-Pro-Azgly-NH₂ acetate [C₅₉H₈₄N₁₈O₁₄.(C₂H₄O₂)ₓ where x=1 to 2.4], goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, lonafarnib, 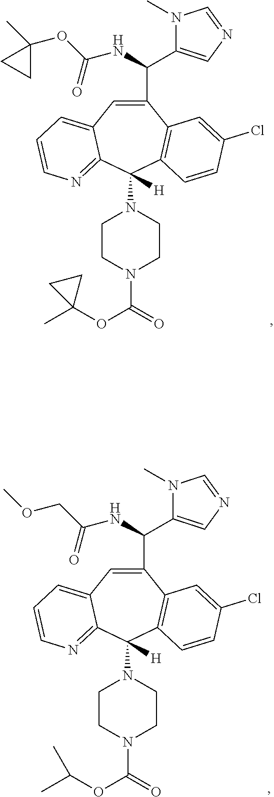
BMS-214662, tipifarnib; amifostine, NVP-LAQ824, suberoyl analide hydroxamic acid, valproic acid, trichostatin A, FK-228, SU11248, sorafenib, KRN951, aminoglutethimide, amsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mercaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, cremophor-free paclitaxel, docetaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, topotecan, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa and darbepoetin alfa.


SEQUENCE LISTING (23 sequences)

SEQ ID NO: 1 - protein, 11 aa, Homo sapiens
RASQSIGSSLH

SEQ ID NO: 2 - protein, 7 aa, Homo sapiens
YASQSLS

SEQ ID NO: 3 - protein, 9 aa, Homo sapiens
HQSSRLPHT

SEQ ID NO: 4 - protein, 5 aa, Homo sapiens
SFAMH

SEQ ID NO: 5 - protein, 10 aa, Homo sapiens
GFTFSSFAMH

SEQ ID NO: 6 - protein, 16 aa, Homo sapiens
VIDTRGATYYADSVKG

SEQ ID NO: 7 - protein, 10 aa, Homo sapiens
LGNFYYGMDV

SEQ ID NO: 8 - DNA, 384 bp, Homo sapiens
  CDS (1)..(384)
atgtcgccatcacaactcattgggtttctgctgctctgggttccagcctccaggggtgaaattgtgctgactcagagcccagactctctgtctgtgactccaggcgagagagtcaccatcacctgccgggccagtcagagcattggtagtagcttacactggtaccagcagaaaccaggtcagtctccaaagcttctcatcaagtatgcatcccagtccctctcaggggtcccctcgaggttcagtggcagtggatctgggacagatttcaccctcaccatcagtagcctcgaggctgaagatgctgcagcgtattactgtcatcagagtagtcgtttacctcacactttcggccaagggaccaaggtggagatcaaacgtacg

SEQ ID NO: 9 - protein, 128 aa, Homo sapiens
MSPSQLIGFLLLWVPASRGEIVLTQSPDSLSVTPGERVTITCRASQSIGSSLHWYQQKPGQSPKLLIKYASQSLSGVPSRFSGSGSGTDFTLTISSLEAEDAAAYYCHQSSRLPHTFGQGTKVEIKRT

SEQ ID NO: 10 - DNA, 384 bp, Homo sapiens
  CDS (1)..(384)
atgtcgccatcacaactcattgggtttctgctgctctgggttccagcctccaggggtgaaattgtgctgactcagagcccagactctctgtctgtgactccaggcgagagagtcaccatcacctgccgggccagtcagagcattggtagtagcttacactggtaccagcagaaaccaggtcagtctccaaagcttctcatcaagtatgcatcccagtccctctcaggggtcccctcgaggttcagtggcagtggatctgggacagatttcaccctcaccatcagtagcctcgaggctgaagatttcgcagtgtattactgtcatcagagtagtcgtttacctcacactttcggccaagggaccaaggtggagatcaaacgtacg

SEQ ID NO: 11 - protein, 128 aa, Homo sapiens
MSPSQLIGFLLLWVPASRGEIVLTQSPDSLSVTPGERVTITCRASQSIGSSLHWYQQKPGQSPKLLIKYASQSLSGVPSRFSGSGSGTDFTLTISSLEAEDFAVYYCHQSSRLPHTFGQGTKVEIKRT

SEQ ID NO: 12 - DNA, 384 bp, Homo sapiens
  CDS (1)..(384)
atgtcgccatcacaactcattgggtttctgctgctctgggttccagcctccaggggtgaaattgtgctgactcagagcccaggtaccctgtctgtgtctccaggcgagagagccaccctctcctgccgggccagtcagagcattggtagtagcttacactggtaccagcagaaaccaggtcaggctccaaggcttctcatcaagtatgcatcccagtccctctcagggatccccgataggttcagtggcagtggatctgggacagatttcaccctcaccatcagtagactggagcctgaagatgctgcagcgtattactgtcatcagagtagtcgtttacctcacactttcggccaagggaccaaggtggagatcaaacgtaca

SEQ ID NO: 13 - protein, 128 aa, Homo sapiens
MSPSQLIGFLLLWVPASRGEIVLTQSPGTLSVSPGERATLSCRASQSIGSSLHWYQQKPGQAPRLLIKYASQSLSGIPDRFSGSGSGTDFTLTISRLEPEDAAAYYCHQSSRLPHTFGQGTKVEIKRT

SEQ ID NO: 14 - DNA, 384 bp, Homo sapiens
  CDS (1)..(384)
atgtcgccatcacaactcattgggtttctgctgctctgggttccagcctccaggggtgaaattgtgctgactcagagcccaggtaccctgtctgtgtctccaggcgagagagccaccctctcctgccgggccagtcagagcattggtagtagcttacactggtaccagcagaaaccaggtcaggctccaaggcttctcatcaagtatgcatcccagtccctctcagggatccccgataggttcagtggcagtggatctgggacagatttcaccctcaccatcagtagactggagcctgaagatttcgcagtgtattactgtcatcagagtagtcgtttacctcacactttcggccaagggaccaaggtggagatcaaacgtaca

SEQ ID NO: 15 - protein, 128 aa, Homo sapiens
MSPSQLIGFLLLWVPASRGEIVLTQSPGTLSVSPGERATLSCRASQSIGSSLHWYQQKPGQAPRLLIKYASQSLSGIPDRFSGSGSGTDFTLTISRLEPEDFAVYYCHQSSRLPHTFGQGTKVEIKRT

SEQ ID NO: 16 - DNA, 411 bp, Homo sapiens
  CDS (1)..(411)
atggagtttgggctgagctgggttttccttgttgctatattaaaaggtgtccagtgtgaggttcagctggtgcagtctgggggaggcttggtaaagcctggggggtccctgagactctcctgtgcagcctctggattcaccttcagtagctttgctatgcactgggttcgccaggctccaggaaaaggtctggagtggatatcagttattgatactcgtggtgccacatactatgcagactccgtgaagggccgattcaccatctccagagacaatgccaagaactccttgtatcttcaaatgaacagcctgagagccgaggacactgctgtgtattactgtgcaagactggggaacttctactacggtatggacgtctggggccaagggaccacggtcaccgtctcctca

SEQ ID NO: 17 - protein, 137 aa, Homo sapiens
MEFGLSWVFLVAILKGVQCEVQLVQSGGGLVKPGGSLRLSCAASGFTFSSFAMHWVRQAPGKGLEWISVIDTRGATYYADSVKGRFTISRDNAKNSLYLQMNSLRAEDTAVYYCARLGNFYYGMDVWGQGTTVTVSS

SEQ ID NO: 18 - DNA, 411 bp, Homo sapiens
  CDS (1)..(411)
atggagtttgggctgagctgggttttccttgttgctatattaaaaggtgtccagtgtgaggttcagctggtgcagtctgggggaggcttggtacagcccggggggtccctgagactctcctgtgcagcctctggattcaccttcagtagctttgctatgcactgggttcgccaggctccaggaaaaggtctggagtggatatcagttattgatactcgtggtgccacatactatgcagactccgtgaagggccgattcaccatctccagagacaatgccaagaactccttgtatcttcaaatgaacagcctgagagccgaggacactgctgtgtattactgtgcaagactggggaacttctactacggtatggacgtctggggccaagggaccacggtcaccgtctcctca

SEQ ID NO: 19 - protein, 137 aa, Homo sapiens
MEFGLSWVFLVAILKGVQCEVQLVQSGGGLVQPGGSLRLSCAASGFTFSSFAMHWVRQAPGKGLEWISVIDTRGATYYADSVKGRFTISRDNAKNSLYLQMNSLRAEDTAVYYCARLGNFYYGMDVWGQGTTVTVSS

SEQ ID NO: 20 - protein, 123 aa, Homo sapiens
VQLLESGGGLVQPGGSLRLSCTASGFTFSSYAMNWVRQAPGKGLEWVSAISGSGGTTFYADSVKGRFTISRDNSRTTYLQMNSLRAEDTAVYYCAKDLGWSDSYYYYYGMDVWGQGTTVTVSS

SEQ ID NO: 21 - protein, 107 aa, Homo sapiens [sequence omitted]

SEQ ID NO: 22 - protein, 117 aa, Artificial Sequence
  heavy chain immunoglobulin
QVQLQESGPGLVKPSETLSLTCTVSGYSISGGYLWNWIRQPPGKGLEWIGYISYDGTNNYKPSLKDRVTISVDTSKNQFSLKLSSVTAADTAVYYCARYGRVFFDYWGQGTLVTVSS

SEQ ID NO: 23 - protein, 112 aa, Artificial Sequence [sequence omitted]